(19) United States
(12) Patent Application Publication
(10) Pub. No.: US 2023/0416396 A1
(43) Pub. Date: Dec. 28, 2023

(54) MULTISPECIFIC ANTIGEN BINDING MOLECULES THAT BIND CD38 AND 4-1BB, AND USES THEREOF

(71) Applicant: Regeneron Pharmaceuticals, Inc., Tarrytown, NY (US)

(72) Inventors: Robert Babb, River Edge, NJ (US); David DiLillo, New York, NY (US); Aynur Hermann, New York, NY (US); Kara Olson, White Plains, NY (US); Yang Shen, Scarsdale, NY (US); Erica Ullman, Yorktown Heights, NY (US)

(21) Appl. No.: 18/319,245

(22) Filed: May 17, 2023

Related U.S. Application Data
(60) Provisional application No. 63/343,441, filed on May 18, 2022.
(60) Provisional application No. 63/478,625, filed on Jan. 5, 2023.

(51) Int. Cl. C07K 16/28; A61P 35/00; A61K 45/06

(52) U.S. Cl. CPC C07K 16/2896; C07K 16/2878; A61P 35/00; C07K 16/2809; A61K 45/06; C07K 16/2818; A61K 2039/505

(57) ABSTRACT

CD38 is expressed on malignant plasma cells. 4-1BB is a costimulatory molecule required for T-cell activation and survival. Provided herein are novel multispecific antigen binding molecules (MABMs) that bind to both CD38 and 4-1BB, for example, to provide “signal 2” in order to enhance the activation of T cells in the presence of a “signal 1”, provided by a Tumor-associated antigen (TAA)×CD3 bispecific antibody or an allogeneic response provided by an antigen presenting cell. In certain embodiments, the multispecific antigen binding molecules of the present invention are capable of inhibiting the growth of tumors expressing CD38. The multispecific antigen binding molecules of the invention are useful for the treatment of diseases and disorders in which an upregulated or induced CD38-targeted immune response is desired and/or therapeutically beneficial. For example, the multispecific antigen binding molecules of the invention are useful for the treatment of various cancers, including multiple myeloma, lymphoma, and leukemia.

### CROSS-REFERENCE TO RELATED APPLICATIONS

This application claims the benefit under 35 U.S.C. § 119(e) of U.S. Provisional Application Nos. 63/343,441, filed May 18, 2022 and 63/478,625, filed Jan. 5, 2023, the disclosures of each herein incorporated by reference in their entireties.

### FIELD OF THE INVENTION

The present invention relates to multispecific antigen binding molecules, which are specific for CD38 and 4-1BB, and methods of use thereof.

### SEQUENCE LISTING

An official copy of the sequence listing is submitted concurrently with the specification electronically via Patent Center. The contents of the electronic sequence listing (10927US01_Sequence_Listing_ST26.xml; Size: 151,552 bytes; and Date of Creation: May 17, 2023) is herein incorporated by reference in its entirety.

### BACKGROUND

Multiple Myeloma (MM) is the second most common blood cancer after non-Hodgkin lymphoma, with a prevalence of ˜120,000, and roughly 30,000 new cases and 13,000 deaths each year in the US. MM is characterized by a clonal expansion of malignant plasma cells which secrete cytokines in an unregulated manner. The production of cytokines, especially IL-6, causes localized organ and tissue damage responsible for many of the symptoms associated with myeloma. Subjects with MM suffer from bone pain and osteoporosis, anemia, impaired kidney function and kidney failure, bacterial infections, and neurological impairments. MM is rarely curable with a median life expectancy of 4-5 years. While progress has been made in treating MM, new therapies have disproportionately benefited younger patients. Prognosis of relapsed MM patients is poor, and novel therapeutic approaches are urgently needed.

CD38, also known as cyclic ADP ribose hydrolase, is a 45 KDa surface glycoprotein expressed on thymocytes, some activated peripheral blood T cells and B cells, plasma cells, and dendritic cells. CD38 functions as an ectoenzyme involved in the metabolism of extracellular nicotinamide adenine dinucleotide (NAD+) and cytoplasmic nicotinamide adenine dinucleotide phosphate (NADP) (Howard, et al. Formation and hydrolysis of cyclic ADP-ribose catalyzed by lymphocyte antigen CD38. Science (1993) 262:1056-9), resulting in the production of Ca²⁺-mobilizing compounds, such as cyclic adenosine diphosphate (ADP) ribose, ADP ribose (ADPR) and nicotinic acid adenine dinucleotide phosphate. Calcium regulation results in the activation of signaling pathways that control a wide range of physiological functions, including lymphocyte proliferation, insulin release by the pancreas, cardiac muscle contraction, neutrophil chemotaxis and T cell activation. CD38 enzymatic activities regulate NAD⁺ levels and improve the function of proteasome inhibitors (Cagnetta, et al. Intracellular NAD(+) depletion enhances bortezomib-induced anti-myeloma activity. Blood (2013) 122:1243-55). In addition, ADPR can be metabolized by CD203a/PC-1 and CD73 to produce the immunosuppressive molecule adenosine (ADO), facilitating the escape of tumor cells from the control of the immune system (Chillemi et al. Roles and modalities of ectonucleotidases in remodeling the multiple myeloma niche. Front Immunol. (2017) 8:305). CD38 appears to contribute to the proliferative potential of B-chronic leukemia/small lymphocytic lymphoma; malignant plasma cells in the bone marrow express high and uniform levels of CD38. Anti-CD38 monoclonal antibodies are thought to deplete CD38+ immunosuppressive cells, such as myeloid-derived suppressor cells, regulatory T cells, and regulatory B cells, leading to increased anti-tumor activity of immune effector cells. Daratumumab, an anti-CD38 antigen binding molecule, has been approved for multiple myeloma patients who are refractory to conventional therapy.

T cell activation involves co-stimulation via the TNF-receptor superfamily and is key to survival, acquisition of effector functions, and memory differentiation. 4-1BB (Tnfrsf9), also known as CD137, is a surface glycoprotein and member of the TNF-receptor superfamily. Receptor expression is induced by lymphocyte activation following TCR-mediated priming, but its levels can be augmented by CD28 co-stimulation. Exposure to ligand or agonist monoclonal antibodies on CD8⁺ T cells costimulates 4-1BB, contributing to the clonal expansion, survival, and development of T cells, induced proliferation in peripheral monocytes, activation of NF-kappaB, enhanced T cell apoptosis induced by TCR/CD3 triggered activation, memory generation, and regulation of CD28 co-stimulation to promote Th1 cell responses. Urelumab (BMS-663513), a fully human IgG4 monoclonal antibody, was the first anti-4-1BB therapeutic to enter clinical trials. Clinical development halted when liver toxicity associated with the antibody was revealed. Utomilumab (PF-05082566) is a humanized IgG2 monoclonal antibody that activates 4-1BB while blocking binding to endogenous 4-1BBL. Thus, a need exists in the art for alternative approaches to treating cancer.

### BRIEF SUMMARY OF THE INVENTION

The present invention relates, in part, to multispecific antigen binding molecules that bind CD38 and 4-1BB and their use in treating various diseases, including cancer.

The multispecific antigen binding molecules can be used alone or in combination with other agents for treating cancers that express CD38.

The multispecific antigen binding molecules provided herein comprise two antigen binding arms, A1 and A2. The A1 arm binds specifically to CD38. The A2 arm comprises a first antigen-binding domain (R1) and a second antigen-binding domain (R2) and A2 binds specifically to 4-1BB. R1 is linked to R2 via a linker, forming stacked antigen-binding domains on the A2 arm. The combination of the A1 arm and the stacked A2 arm is termed a 1+2 format. The antigen-binding domains of A2 may be contained in Fabs. See FIG. 1. In some aspects, R1 and R2 bind different 4-1BB epitopes. In some aspects, R1 and R2 bind the same 4-1BB epitopes. In some aspects, the amino acid sequence of R1 and R2 heavy chain variable regions are identical or substantially similar, i.e., less than 5, or less than 4, or less than 3, amino acid differences in a heavy chain variable region or a heavy chain complementarity determining region, or 2 or 1 amino acid differences in a heavy chain variable region or a heavy chain complementarity determining region. In some aspects, the R1 and R2 heavy chain variable regions are different, i.e., have different antigen binding sequences. In some embodiments, R1 is comprised in a first Fab (Fab2) and R2 is comprised in a second Fab (Fab3). The Fab2 and Fab3 of the anti-CD38×anti-4-1BB 1+2 construct are connected via a linker from the N-terminus of the VH-2 4-11BB “IN” Fab2 to the C-terminus of the CH1-3 “OUT” Fab3.

### Multispecific Antigen Binding Molecules Comprising Anti-CD38 and Anti-4-1BB Antigen Binding Domains

The present disclosure provides multispecific antigen binding molecules that bind CD38 and 4-1BB. Such multispecific antigen binding molecules are also referred to herein as “anti-CD38/anti-4-1BB multispecific antigen binding molecules”. The CD38 antigen binding arm A1 comprises one antigen binding domain. The 4-1BB antigen binding arm A2 comprises two antigen binding domains, R1 and R2. The R1 is referred to herein as the 4-1BB “IN” binding domain, and the R2 is referred to herein as the 4-1BB “OUT” domain. The multispecific antigen binding molecules having the stacked antigen-binding domains are referred to herein as anti-CD38/anti-4-1BB 1+2 multispecific antigen binding molecules, or anti-CD38×anti-4-1BB 1+2 multispecific antigen binding molecules, etc.

The anti-CD38 portion of the anti-CD38/anti-4-1BB multispecific molecule is useful for targeting tumor cells that express CD38 (e.g., plasma cells), and the anti-4-1BB portion of the multispecific molecule is useful for providing co-stimulation of T cells activated by cognate MHC peptide or tumor targeted CD3 multispecific antigen binding molecules. The simultaneous binding of CD38 on a tumor cell and 4-1BB on a T-cell facilitates directed killing (cell lysis) of the targeted tumor cell by the activated T-cell. The anti-CD38/anti-4-1BB 1+2 multispecific molecules provided herein are therefore useful, inter alia, for treating diseases and disorders related to or caused by CD38-expressing tumors (e.g., lymphomas, leukemias, multiple myeloma, hepatocellular carcinoma, non-small cell lung cancer, melanoma, pancreatic ductal adenocarcinoma, glioma, or breast cancer).

The multispecific antigen binding molecules provided herein comprise a first antigen binding arm A1 comprising an antigen binding domain, that specifically binds human CD38, and a second antigen binding arm A2 comprising two antigen binding domains, R1 and R2, that specifically bind 4-1BB. The present disclosure includes anti-CD38/anti-4-1BB 1+2 multispecific molecules (e.g., multispecific antigen binding molecules) wherein each antigen binding domain, comprises a heavy chain variable region (HCVR) paired with a light chain variable region (LCVR). In certain exemplary embodiments of the invention, the anti-CD38 antigen binding domain and the anti-4-1BB antigen binding domains each comprise different, distinct HCVRs paired with a common LCVR or universal LCVR. For example, as illustrated in Example 4 herein, multispecific antigen binding molecules were constructed comprising a first antigen binding domain that specifically binds CD38, wherein the first antigen binding domain comprises an HCVR derived from an anti-CD38 antigen binding molecule; and a second antigen binding domain (R1) and a third antigen binding domain (R2) that specifically bind 4-1BB, wherein the second antigen binding domain (R1) and third antigen binding domain (R2) each comprise an HCVR derived from an anti-4-1BB antigen binding molecule, where each HCVR is paired with a universal light chain LCVR. In such embodiments, the first, second, and third antigen binding domains comprise distinct anti-CD38 and anti-4-1BB HCVRs, respectively, but share a common light chain LCVR.

Provided herein are bispecific antigen-binding molecules comprising:
(a) a first antigen-binding arm comprising three CDRs of a heavy chain variable region (HCVR) and three CDRs of a LCVR, wherein the first antigen-binding arm binds specifically to CD38; and
(b) a second antigen-binding arm comprising a first antigen-binding region (R1) comprising three CDRs of a HCVR (R1-HCVR) and three CDRs of a LCVR (R1-LCVR); and a second antigen-binding region (R2) comprising three CDRs of a HCVR (R2-HCVR) and three CDRs of a LCVR (R2-LCVR), wherein the second antigen-binding arm binds specifically to 4-1BB.

In some aspects, R1 and R2 bind to the same epitope on 4-1BB. In some aspects, R1 and R2 bind to different epitopes on 4-1BB.

In some aspects, R1 and R2 are connected via a peptide linker. An exemplary peptide linker comprises a peptide sequence of (GGGGS)n, wherein n is 1 to 6.

In some aspects, the bispecific antigen-binding molecule comprises a first antigen-binding arm, wherein the first antigen-binding arm comprises three CDRs of a HCVR comprising an amino acid sequence selected from the group consisting of SEQ ID NOs: 2 and 40.

In some aspects, the bispecific antigen-binding molecule comprises a first antigen-binding arm, wherein the first antigen-binding arm comprises three CDRs of a LCVR comprising an amino acid sequence selected from the group consisting of SEQ ID NOs: 18 and 48.

In some aspects, the bispecific antigen-binding molecule comprises a first antigen-binding arm, wherein the first antigen-binding arm comprises three heavy chain complementarity determining regions (HCDR1-HCDR2-HCDR3) comprising an amino acid sequence selected from the group consisting of SEQ ID NOs: 4-6-8, and 42-44-46, respectively.

In some aspects, the bispecific antigen-binding molecule comprises a first antigen-binding arm, wherein the first antigen-binding arm comprises three light chain complementarity determining regions (LCDR1-LCDR2-LCDR3) comprising an amino acid sequence selected from the group consisting of SEQ ID NOs: 20-22-24, and 50-52-54, respectively.

In some aspects, the bispecific antigen-binding molecule comprises a first antigen-binding arm, wherein the first antigen-binding arm comprises a HCVR comprising the amino acid sequence selected from the group consisting of SEQ ID NOs: 2 and 40.

In some aspects, the bispecific antigen-binding molecule comprises a first antigen-binding arm, wherein the first antigen-binding arm comprises a LCVR comprising the amino acid sequence selected from the group consisting of SEQ ID NOs: 18 and 48.

In some aspects, the bispecific antigen-binding molecule comprises a first antigen-binding arm, wherein the first antigen-binding arm comprises a HCVR comprising the amino acid sequence selected from the group consisting of SEQ ID NOs: 2 and 40; and a LCVR comprising the amino acid sequence selected from the group consisting of SEQ ID NOs: 18 and 48.

In some aspects, the bispecific antigen-binding molecule comprises a first antigen-binding arm, wherein the second antigen-binding arm comprises a first antigen-binding region (R1); and a second antigen-binding region (R2).

In some aspects, R1 comprises three CDRs of a HCVR (R1-HCVR) comprising an amino acid sequence selected from the group consisting of SEQ ID NOs: 10, 32, 62, 72, 86 and 94.

In some aspects, R1 comprises three CDRs of a LCVR (R1-LCVR) comprising an amino acid sequence selected from the group consisting of SEQ ID NOs: 18 and 48.

In some aspects, R1 comprises three heavy chain complementarity determining regions (R1-HCDR1-R1-HCDR2-R1-HCDR3) comprising an amino acid sequence selected from the group consisting of SEQ ID NOs: 12-14-16, 34-36-38, 64-66-68, 74-76-78, 88-90-92, and 96-98-100, respectively.

In some aspects, R1 comprises three light chain complementarity determining regions (R1-LCDR1-R1-LCDR2-R1-LCDR3) comprising an amino acid sequence selected from the group consisting of SEQ ID NOs: 20-22-24, and 50-52-54, respectively.

In some aspects, R1 comprises a HCVR (R1-HCVR) comprising the amino acid sequence selected from the group consisting of SEQ ID NOs: 10, 32, 62, 72, 86 and 94.

In some aspects, R1 comprises a LCVR (R1-LCVR) comprising the amino acid sequence selected from the group consisting of SEQ ID NOs: 18 and 48.

In some aspects, R1 comprises a R1-HCVR comprising the amino acid sequence selected from the group consisting of SEQ ID NOs: 10, 32, 62, 72, 86 and 94; and a R1-LCVR comprising the amino acid sequence selected from the group consisting of SEQ ID NOs: 18 and 48.

In some aspects, R2 comprises three CDRs of a HCVR (R2-HCVR) comprising the amino acid sequence selected from the group consisting of SEQ ID NOs: 10, 32, 62, 72, 86 and 94.

In some aspects, R2 comprises three CDRs of a LCVR (R2-LCVR) comprising the amino acid sequence selected from the group consisting of SEQ ID NOs: 18 and 48.

In some aspects, R2 comprises three heavy chain complementarity determining regions (R2-HCDR1-R2-HCDR2-R2-HCDR3) comprising an amino acid sequence selected from the group consisting of SEQ ID NOs: 12-14-16, 34-36-38, 64-66-68, 74-76-78, 88-90-92, and 96-98-100, respectively.

In some aspects, R2 comprises three light chain complementarity determining regions (R2-LCDR1-R2-LCDR2-R2-LCDR3) comprising an amino acid sequence selected from the group consisting of SEQ ID NOs: 20-22-24, and 50-52-54, respectively.

In some aspects, R2 comprises a HCVR (R2-HCVR) comprising the amino acid sequence selected from the group consisting of SEQ ID NOs: 10, 32, 62, 72, 86 and 94.

In some aspects, R2 comprises a LCVR (R2-LCVR) comprising the amino acid sequence selected from the group consisting of SEQ ID NOs: 18 and 48.

In some aspects, R2 comprises a R2-HCVR comprising the amino acid sequence selected from the group consisting of SEQ ID NOs: 10, 32, 62, 72, 86 and 94; and a R2-LCVR comprising the amino acid sequence selected from the group consisting of SEQ ID NOs: 18 and 48.

In some aspects, the bispecific antigen-binding molecule comprises:
(a) a first antigen-binding arm comprising three CDRs of a HCVR comprising the amino acid sequence of SEQ ID NO: 40, and three CDRs of a LCVR comprising the amino acid sequence of SEQ ID NO: 48; and
(b) a second antigen-binding arm comprising:
   (i) a first antigen-binding region (R1) comprising three CDRs of R1-HCVR comprising the amino acid sequence selected from the group consisting of SEQ ID NOs: 32, 62 and 72; and three CDRs of R1-LCVR comprising the amino acid sequence of SEQ ID NO: 48; and
   (ii) a second antigen-binding region (R2) comprising three CDRs of R2-HCVR comprising the amino acid sequence selected from the group consisting of SEQ ID NOs: 62, 84 and 94; and three CDRs of R2-LCVR comprising the amino acid sequence of SEQ ID No: 48.

In some aspects, the bispecific antigen-binding molecule comprises:
(a) a first antigen-binding arm comprising HCDR1-HCDR2-HCDR3-LCDR1-LCDR2-LCDR3 comprising amino acid sequences selected from the group consisting of SEQ ID NOs: 42-44-46-50-52-54, respectively; and
(b) a second antigen-binding arm comprising:
   (i) a first antigen-binding region (R1) comprising R1-HCDR1-R1-HCDR2-R1-HCDR3-R1-LCDR1-R1-LCDR2-R1-LCDR3 comprising amino acid sequences selected from the group consisting of SEQ ID NOs: 34-36-38-50-52-54, 64-66-68-50-52-54, and 74-76-78-50-52-54, respectively; and
   (ii) a second antigen-binding region (R2) comprising R2-HCDR1-R2-HCDR2-R2-HCDR3-R2-LCDR1-R2-LCDR2-R2-LCDR3 comprising amino acid sequences selected from the group consisting of SEQ ID NOs: 64-66-68-50-52-54, 74-76-78-50-52-54, and 88-90-92-50-52-54, respectively.

In some aspects, the bispecific antigen-binding molecule comprises:
(a) a first antigen-binding arm comprising a HCVR comprising the amino acid sequence of SEQ ID NO: 40 and a LCVR comprising the amino acid sequence of SEQ ID NO: 48; and (b) a second antigen-binding arm comprising:
   (i) a first antigen-binding region (R1) comprising R1-HCVR comprising the amino acid sequence selected from the group consisting of SEQ ID NOs: 32, 62 and 72; and R1-LCVR comprising the amino acid sequence of SEQ ID NO: 48; and
   (ii) a second antigen-binding region (R2) comprising R2-HCVR comprising the amino acid sequence selected from the group consisting of SEQ ID NO: 62, 86 and 94; and R2-LCVR comprising the amino acid sequence of SEQ ID NO: 48.

In some aspects, the bispecific antigen-binding molecule comprises:
(a) a first antigen-binding arm comprising a HCVR comprising the amino acid sequence of SEQ ID NO: 40 and a LCVR comprising the amino acid sequence of SEQ ID NO: 48; and (b) a second antigen-binding arm comprising:
   (i) a first antigen-binding region (R1) comprising R1-HCVR comprising the amino acid sequence of SEQ ID NOs: 32; and R1-LCVR comprising the amino acid sequence of SEQ ID NO: 48; and
   (ii) a second antigen-binding region (R2) comprising R2-HCVR comprising the amino acid sequence of SEQ ID NO: 86; and R2-LCVR comprising the amino acid sequence of SEQ ID NO: 48.

In some aspects, the bispecific antigen-binding molecule comprises:
(a) a first antigen-binding arm comprising a HCVR comprising the amino acid sequence of SEQ ID NO: 40 and a LCVR comprising the amino acid sequence of SEQ ID NO: 48; and (b) a second antigen-binding arm comprising:
   (i) a first antigen-binding region (R1) comprising R1-HCVR comprising the amino acid sequence of SEQ ID NOs: 72; and R1-LCVR comprising the amino acid sequence of SEQ ID NO: 48; and
   (ii) a second antigen-binding region (R2) comprising R2-HCVR comprising the amino acid sequence of SEQ ID NO: 94; and R2-LCVR comprising the amino acid sequence of SEQ ID NO: 48.

In some aspects, the bispecific antigen-binding molecule comprises:
(a) a first antigen-binding arm comprises a HCVR comprising the amino acid sequence of SEQ ID NO: 40 and a LCVR comprising the amino acid sequence of SEQ ID NO: 48; and (b) second antigen-binding arm comprises:
   (i) a first antigen-binding region (R1) comprising R1-HCVR comprising the amino acid sequence of SEQ ID NOs: 62; and R1-LCVR comprising the amino acid sequence of SEQ ID NO: 48; and
   (ii) a second antigen-binding region (R2) comprising R2-HCVR comprising the amino acid sequence of SEQ ID NO: 62; and R2-LCVR comprising the amino acid sequence of SEQ ID NO: 48.

In some aspects, the bispecific antigen-binding molecule is a bispecific antibody.

In some aspects, the bispecific antigen-binding molecule is a bispecific antibody comprising a heavy chain constant region of IgG1 or IgG4 isotype.

In some aspects, the bispecific antibody comprises a first heavy chain comprising the HCVR of the first antigen-binding arm, and a second heavy chain comprising R1-HCVR and R2-HCVR of the second antigen-binding arm, wherein the second heavy chain comprises the mutations H435R and Y436F (EU numbering).

In some aspects, the bispecific antibody comprises a first heavy chain comprising the HCVR of the first antigen-binding arm paired with a light chain comprising the LCVR of the first antigen-binding arm, wherein the first heavy chain comprises the amino acid sequence of SEQ ID NO: 58 and the light chain comprises the amino acid sequence of SEQ ID NO: 60.

In some aspects, the bispecific antibody comprises a second heavy chain comprising R1-HCVR and R2-HCVR of the second antigen-binding arm paired with a first light chain comprising R1-LCVR and a second light chain comprising R2-LCVR, wherein the second heavy chain comprises the amino acid sequence selected from the group consisting of SEQ ID NO: 56, 70, 80, 82, and 84; the first light chain comprises the amino acid sequence of SEQ ID NO: 60, and the second light chain comprises the amino acid sequence of SEQ ID NO: 60.

In some aspects, the bispecific antibody comprises:
(a) a first antigen-binding arm that specifically binds human CD38 comprising a heavy chain comprising the sequence of SEQ ID NO: 58, and a light chain comprising the sequence of SEQ ID NO: 60; and
(b) a second antigen-binding arm that specifically binds human 4-1BB comprising a heavy chain comprising the sequence of SEQ ID NO: 56, a first light chain comprising the sequence of SEQ ID NO: 60, and a second light chain comprising the sequence of SEQ ID NO: 60.

In some aspects, the bispecific antibody comprises:
(a) a first antigen-binding arm that specifically binds human CD38 comprising a heavy chain comprising the sequence of SEQ ID NO: 58, and a light chain comprising the sequence of SEQ ID NO: 60; and
(b) a second antigen-binding arm that specifically binds human 4-1BB comprising a heavy chain comprising the sequence of SEQ ID NO: 70, a first light chain comprising the sequence of SEQ ID NO: 60, and a second light chain comprising the sequence of SEQ ID NO: 60.

In some aspects, the bispecific antibody comprises:
(a) a first antigen-binding arm that specifically binds human CD38 comprising a heavy chain comprising the sequence of SEQ ID NO: 58, and a light chain comprising the sequence of SEQ ID NO: 60; and
(b) a second antigen-binding arm that specifically binds human 4-1BB comprising a heavy chain comprising the sequence of SEQ ID NO: 80, a first light chain comprising the sequence of SEQ ID NO: 60, and a second light chain comprising the sequence of SEQ ID NO: 60.

In some aspects, the bispecific antibody comprises:
(a) a first antigen-binding arm that specifically binds human CD38 comprising a heavy chain comprising the sequence of SEQ ID NO: 58, and a light chain comprising the sequence of SEQ ID NO: 60; and
(b) a second antigen-binding arm that specifically binds human 4-1BB comprising a heavy chain comprising the sequence of SEQ ID NO: 82, a first light chain comprising the sequence of SEQ ID NO: 60, and a second light chain comprising the sequence of SEQ ID NO: 60.

In some aspects, the bispecific antibody comprises:
(a) a first antigen-binding arm that specifically binds human CD38 comprising a heavy chain comprising the sequence of SEQ ID NO: 58, and a light chain comprising the sequence of SEQ ID NO: 60; and
(b) a second antigen-binding arm that specifically binds human 4-1BB comprising a heavy chain comprising the sequence of SEQ ID NO: 84, a first light chain comprising the sequence of SEQ ID NO: 60, and a second light chain comprising the sequence of SEQ ID NO: 60.

Provided herein is a bispecific antigen-binding molecule comprising a first antigen binding arm that binds specifically to CD38 and a second antigen-binding arm that binds specifically to 4-1BB, wherein:
(a) the first antigen binding arm comprises three CDRs of a HCVR comprising the amino acid sequence of SEQ ID NO: 40, and three CDRs of LCVR comprising the amino acid sequence of SEQ ID NO: 48; and
(b) the second antigen-binding arm comprises:
   (i) a first antigen-binding region (R1) comprising three CDRs of a HCVR (R1-HCVR) comprising the amino acid sequence of SEQ ID NO: 62, and three CDRs of a LCVR (R1-LCVR) comprising the amino acid sequence of SEQ ID NO: 48; and
   (ii) a second antigen-binding region (R2) comprising three CDRs of a HCVR (R2-HCVR) comprising the amino acid sequence of SEQ ID NO: 62, and three CDRs of a LCVR (R2-LCVR) comprising the amino acid sequence of SEQ ID NO: 48.

In some aspects, the bispecific antigen-binding molecule is a bispecific antibody.

In some aspects, the bispecific antibody comprises a first heavy chain comprising the HCVR of the first antigen-binding arm, wherein the first heavy chain is paired with a light chain comprising the LCVR of the first antigen-binding arm, wherein the first heavy chain comprises the amino acid sequence of SEQ ID NO: 58 and the light chain comprises the amino acid sequence of SEQ ID NO: 60. In some aspects, the bispecific antibody comprises a second heavy chain comprising R1-HCVR and R2-HCVR of the second antigen-binding arm, wherein the second heavy chain is paired with a first light chain comprising R1-LCVR, and a second light chain comprising R2-LCVR, wherein the second heavy chain comprises the amino acid sequence of SEQ ID NO: 70, 82 or 84, the first light chain comprises the amino acid sequence of SEQ ID NO: 60, and the second light chain comprises the amino acid sequence of SEQ ID NO: 60.

In some aspects, the bispecific antigen-binding molecule comprises:
(a) a first antigen-binding arm that specifically binds human CD38 comprising a heavy chain comprising the sequence of SEQ ID NO: 58, and a light chain comprising the sequence of SEQ ID NO: 60; and
(b) a second antigen-binding arm that specifically binds human 4-1BB comprising: (i) a heavy chain comprising the sequence of SEQ ID NO: 70, a first light chain comprising the sequence of SEQ ID NO: 60, and a second light chain comprising the sequence of SEQ ID NO: 60;
   (ii) a heavy chain comprising the sequence of SEQ ID NO: 82, a first light chain comprising the sequence of SEQ ID NO: 60, and a second light chain comprising the sequence of SEQ ID NO: 60; or
   (iii) a heavy chain comprising the sequence of SEQ ID NO: 84, a first light chain comprising the sequence of SEQ ID NO: 60, and a second light chain comprising the sequence of SEQ ID NO: 60.

In some embodiments, the multispecific antigen binding molecule inhibits the proliferation of CD38+ tumor cells selected from the group consisting of myeloma cells, leukemia cells, lymphoma cells, hepatocellular carcinoma cells, non-small cell lung cancer cells, melanoma cells, pancreatic ductal adenocarcinoma cells, glioma cells, or breast cancer cells.

In another aspect, pharmaceutical compositions comprising the multispecific antigen binding molecule and a pharmaceutically acceptable carrier or diluent are provided. In a related aspect, the invention features a composition which is a combination of an anti-CD38/anti-4-1BB 1+2 multispecific antigen binding molecule and a second therapeutic agent. In one embodiment, the second therapeutic agent is any agent that is advantageously combined with an anti-CD38/anti-4-1BB 1+2 multispecific antigen binding molecule.

In another aspect, nucleic acid molecules comprising a nucleotide sequence encoding the any one of the A1, or A2 are provided. In some aspects, nucleic acid molecules comprising a nucleotide sequence encoding the any one of the HCVR, LCVR, HCDR1, HCDR2, HCDR3, LCDR1, LCDR2, LCDR3, heavy chain, and/or light chain are provided. In some embodiments, the nucleic acid molecule comprises one or more nucleotide sequences set forth in Tables 2, 4, 6, 8, or 10 are provided. The nucleic acid molecules comprising the nucleic acid sequences can be in any functional combination or arrangement thereof.

In some aspects, provided herein is an isolated nucleic acid molecule comprising a nucleic acid sequence encoding a heavy chain variable region (HCVR) of a multispecific antigen binding molecule antigen binding arm A1, wherein A1 binds to CD38, and wherein (a) the HCVR comprises three heavy chain complementarity determining regions (HCDR1-HCDR2-HCDR3) comprising the amino acid sequence of SEQ ID NOs: 42, 44, and 46, respectively or (b) the HCVR comprises an amino acid sequence of SEQ ID NO: 40.

In some aspects, provided herein is an isolated nucleic acid molecule comprising a nucleic acid sequence encoding a heavy chain of a multispecific antigen binding molecule antigen binding arm A1, wherein A1 binds to CD38, and wherein the heavy chain comprises an amino acid sequence of SEQ ID NO: 58.

In some aspects, provided herein is an isolated nucleic acid molecule comprising a nucleic acid sequence encoding heavy chain variable regions (HCVR) of a multispecific antigen binding molecule antigen binding arm A2, wherein A2 comprises a first antigen-binding domain (R1) that binds to 4-1BB and a second antigen-binding domain (R2) that binds to 4-1BB, and wherein (a) R1-HCVR comprises three heavy chain complementarity determining regions (HCDR1, HCDR2, and HCDR3) comprising the amino acid sequence of SEQ ID NOs: 34, 36, and 38, and the R2-HCVR comprises three heavy chain complementarity determining regions (HCDR1, HCDR2, and HCDR3) comprising the amino acid sequence of SEQ ID NOs: 88, 90, and 92, or (b) the R1-HCVR comprises an amino acid sequence of SEQ ID NO: 32 and R2-HCVR comprises an amino acid sequence of SEQ ID NO: 86.

Provided herein is an isolated nucleic acid molecule comprising a nucleic acid sequence encoding a heavy chain of a multispecific antigen binding molecule antigen binding arm A2, wherein A2 binds to 4-1BB, and wherein the heavy chain comprises an amino acid sequence of SEQ ID NO: 56.

Also provided herein is an isolated nucleic acid molecule comprising a nucleic acid sequence encoding a first heavy chain variable region (HCVR) and a second HCVR of a multispecific antigen binding molecule antigen binding arm A2, wherein A2 binds to 4-1BB, and wherein (a) the first HCVR comprises three heavy chain complementarity determining regions (HCDR1, HCDR2, and HCDR3) comprising the amino acid sequence of SEQ ID NOs: 64, 66, and 68, and the second HCVR comprises three heavy chain complementarity determining regions (HCDR1, HCDR2, and HCDR3) comprising the amino acid sequence of SEQ ID NOs: 64, 66, and 68, or (b) the first HCVR comprises an amino acid sequence of SEQ ID NO: 62 and the second HCVR comprises an amino acid sequence of SEQ ID NO: 62.

Also provided herein is an isolated nucleic acid molecule comprising a nucleic acid sequence encoding a heavy chain of a multispecific antigen binding molecule antigen binding arm A2, wherein A2 binds to 4-1BB, and wherein the heavy chain comprises an amino acid sequence selected from the group consisting of SEQ ID NOs: 70, 82, and 84.

Provided herein is an isolated nucleic acid molecule comprising a nucleic acid sequence encoding a first heavy chain variable region (HCVR) and a second HCVR of a multispecific antigen binding molecule antigen binding arm A2, wherein A2 binds to 4-1BB, and wherein (a) the first HCVR comprises three heavy chain complementarity determining regions (HCDR1, HCDR2, and HCDR3) comprising the amino acid sequence of SEQ ID NOs: 74, 76, and 78, and the second HCVR comprises three heavy chain complementarity determining regions (HCDR1, HCDR2, and HCDR3) comprising the amino acid sequence of SEQ ID NOs: 96, 98, and 100, or (b) the first HCVR comprises an amino acid sequence of SEQ ID NO: 72 and the second HCVR comprises an amino acid sequence of SEQ ID NO: 94.

Provided herein is an isolated nucleic acid molecule comprising a nucleic acid sequence encoding a heavy chain of a multispecific antigen binding molecule antigen binding arm A2, wherein A2 binds to 4-1BB, and wherein the heavy chain comprises an amino acid sequence of SEQ ID NO: 80.

Also provided herein is an isolated nucleic acid molecule comprising a nucleic acid sequence encoding a light chain variable region (LCVR) of a multispecific antigen binding molecule, wherein (a) the LCVR comprises three heavy chain complementarity determining regions (LCDR1, LCDR2, and LCDR3) comprising the amino acid sequence of SEQ ID NOs: 50, 52, and 54, or (b) the LCVR comprises an amino acid sequence of SEQ ID NO: 48.

And provided herein is an isolated nucleic acid molecule comprising a nucleic acid sequence encoding a light chain of a multispecific antigen binding molecule, wherein the light chain comprises an amino acid sequence of SEQ ID NO: 60.

Provided herein is an expression vector or a set of expression vectors comprising one more nucleic acid molecules of any one of the nucleic acids described above. Also provided is a host cell comprising one or more expression vectors provided herein. In some aspects, the host cell is a mammalian cell or a prokaryotic cell. In some aspects, the host cell is a Chinese Hamster Ovary (CHO) cell or an Escherichia coli (E. coli) cell. Further provided are compositions comprising one or more nucleic acid molecules described herein.

In some embodiments, methods are provided for producing a multispecific antigen binding molecule. As provided herein, the method comprises growing a host cell under conditions permitting production of the multispecific antigen binding molecule, wherein the host cell comprises a nucleic acid molecule comprising a nucleic acid sequence encoding a heavy chain variable region (HCVR) of a multispecific antigen binding molecule antigen binding arm A1, a nucleic acid molecule comprising a nucleic acid sequence encoding heavy chain variable regions (HCVRs) of a multispecific antigen binding molecule antigen binding arm A2, and/or a nucleic acid molecule comprising a nucleic acid sequence encoding a common light chain variable region (LCVR). In some aspects, each nucleic acid molecule is in the same expression vector. In some aspects, one or more of the nucleic acid molecules are in different expression vectors. In some aspects, the host cell comprises a nucleic acid molecule comprising a nucleic acid sequence encoding a heavy chain of the multispecific antigen binding molecule antigen binding arm A1, a nucleic acid molecule encoding a heavy chain of the multispecific antigen binding molecule antigen binding arm A2, and/or a nucleic acid molecule comprising a nucleic acid sequence encoding a common light chain. In some aspects, each nucleic acid molecule is in the same expression vector. In some aspects, one or more of the nucleic acid molecules are in different expression vectors.

Provided herein are methods of inhibiting growth of a plasma cell tumor in a subject, comprising administering any one or more of the multispecific antigen binding molecules described herein, or pharmaceutical compositions provided herein, to the subject. In some aspects, the plasma cell tumor is multiple myeloma.

Also provided herein is the use of a multispecific antigen binding molecule described herein, or pharmaceutical composition provided herein, in the manufacture of a medicament for inhibiting growth of a plasma cell tumor in a subject. The multispecific antigen binding molecule or pharmaceutical composition can be administered to the subject. In some aspects, the plasma cell tumor is multiple myeloma.

Provided herein are methods of inhibiting growth of a tumor in a subject, the method comprising administering any one or more of the multispecific antigen binding molecules described herein, or pharmaceutical compositions provided herein, to the subject. In some aspects, the tumor is selected from the group consisting of multiple myeloma, lymphoma, B-cell leukemia, hepatocellular carcinoma, non-small cell lung cancer, melanoma, pancreatic ductal adenocarcinoma, glioma, or breast cancer, or another cancer characterized in part by having CD38+ cells.

Also provided herein is the use of a multispecific antigen binding molecule described herein, or pharmaceutical composition provided herein, in the manufacture of a medicament for inhibiting growth of a tumor in a subject. The multispecific antigen binding molecule or pharmaceutical composition can be administered to the subject. In some aspects, the tumor is selected from the group consisting of multiple myeloma, lymphoma, B-cell leukemia, hepatocellular carcinoma, non-small cell lung cancer, melanoma, pancreatic ductal adenocarcinoma, glioma, or breast cancer, or another cancer characterized in part by having CD38+ cells.

Provided herein is a method of treating a patient suffering from multiple myeloma, or from another BCMA-expressing B cell malignancy comprising administering a multispecific antigen binding molecule provided herein, or a pharmaceutical composition provided herein, to the subject. In some aspects, the BCMA-expressing B cell malignancy is selected from the group consisting of Waldenström's macroglobulinemia, Burkitt's lymphoma, Diffuse Large B-Cell lymphoma, Non-Hodgkin's lymphoma, chronic lymphocytic leukemia, follicular lymphoma, mantle cell lymphoma, marginal zone lymphoma, lymphoplasmacytic lymphoma, and Hodgkin's lymphoma.

Also provided herein is the use of a multispecific antigen binding molecule described herein, or pharmaceutical composition provided herein, in the manufacture of a medicament for treating a patient suffering from multiple myeloma, or from another BCMA-expressing B cell malignancy. The multispecific antigen binding molecule or pharmaceutical composition can be administered to the subject.

Provided herein is a method of treating a patient suffering from a CD38+ tumor and/or a BCMA-expressing tumor. The method comprises administering a multispecific antigen binding molecule described herein, or a pharmaceutical composition described herein, to the subject in combination with an anti-PD-1 antibody or antigen binding fragment thereof. In some aspects, the anti-PD-1 antibody or antigen binding fragment is an anti-PD-1 antibody, for example, cemiplimab (bsAb2810).

Also provided herein is the use of a multispecific antigen binding molecule described herein, or pharmaceutical composition provided herein, in the manufacture of a medicament for treating a patient suffering from a CD38+ tumor and/or a BCMA-expressing tumor.

In some embodiments, the methods or uses provided herein further comprise administering a second therapeutic agent or therapeutic regimen. In some aspects, the second therapeutic is an antibody that binds plasma cell tumors. In some aspects, the second therapeutic is an anti-BCMA/anti-CD3 bispecific antigen binding molecule. In some aspects, the second therapeutic is an anti-CD20/anti-CD3 bispecific antigen binding molecule. In some aspects, the second therapeutic is an anti-CD28/anti-4-1BB bispecific antigen binding molecule.

In some aspects, the second therapeutic agent or therapeutic regimen comprises a chemotherapeutic drug, DNA alkylators, immunomodulators, proteasome inhibitors, histone deacetylase inhibitors, radiotherapy, a stem cell transplant, an oncolytic virus, a cancer vaccine, an immunocytokine, a CAR-T cell, a different bispecific antibody that interacts with a different tumor cell surface antigen and a T cell or immune cell antigen, an antibody drug conjugate, a bispecific antibody conjugated to an anti-tumor agent, a PD-1 inhibitor, a PD-L1 inhibitor, a CTLA-4 checkpoint inhibitor, a CD22 inhibitor, a BCMA inhibitor, a CD28 agonist, a CD20 inhibitor, or combinations thereof.

Further provided are uses of a multispecific antigen binding molecule provided herein, or a pharmaceutical composition provided herein, in the treatment of a disease or disorder associated with expression of CD38, CD20, and/or BCMA. In some aspects, the disease or disorder is cancer. In some aspects, the cancer is multiple myeloma. In some aspects, the multispecific antigen binding molecule or pharmaceutical composition is for use in combination with an anti-PD-1 antibody or antigen binding fragment thereof. The multispecific antigen binding molecule, or pharmaceutical composition comprising the multispecific antigen binding molecule, is injected intravenously, intramuscularly or subcutaneously.

Provided herein are anti-CD38/anti-4-1BB 1+2 multispecific antigen binding molecules having a modified glycosylation pattern. In some applications, modification to remove undesirable glycosylation sites may be useful, or an antigen binding molecule lacking a fucose moiety present on the oligosaccharide chain, for example, to increase antigen binding molecule dependent cellular cytotoxicity (ADCC) function (see Shield et al. (2002) JBC 277:26733). In other applications, modification of galactosylation can be made in order to modify complement dependent cytotoxicity (CDC).

In yet another aspect, provided herein are therapeutic methods for targeting/killing tumor cells expressing CD38 using an anti-CD38/anti-4-1BB multispecific antigen binding molecule of the invention, wherein the therapeutic methods comprise administering a therapeutically effective amount of a pharmaceutical composition comprising an anti-CD38/anti-4-1BB multispecific antigen binding molecule provided herein to a subject in need thereof.

The present disclosure also includes the use of an anti-CD38/anti-4-1BB multispecific antigen binding molecule provided herein in the manufacture of a medicament for the treatment of a disease or disorder related to or caused by CD38 expression.

Other embodiments will become apparent from a review of the ensuing detailed description.

### BRIEF DESCRIPTION OF THE FIGURES

FIG. 1 is a schematic depicting the structure of an exemplary anti-CD38×anti-4-11BB 1+2 multispecific antigen binding molecule. The A1 antigen binding arm comprises Fab1, which is specific for CD38, while the A2 antigen binding arm comprises two Fabs, Fab2 and Fab3, each of which are specific to 4-1BB. The Fab2 and Fab3 of the anti-CD38×anti-4-1BB 1+2 construct are connected via a linker from the N-terminus of the VH-2 4-11BB “IN” Fab2 to the C-terminus of the CH1-3 “OUT” Fab3. The Fab1, Fab2, and Fab3 light chains comprise VL-1, VL-2, and VL-3 universal light chains, respectively.

FIG. 2A compares percent activation of 4-1BB in the Jurkat reporter assay by multispecific antigen binding molecules in various formats, including identical split 4-1BB Fabs, identical stacked 4-1BB Fabs, and mixed stacked 4-1BB Fabs. FIG. 2B illustrates the various constructs tested in the screen, and depicts the target dependent bioassay.

FIG. 3 illustrates in vivo tumor burden over time after administration of human multiple myeloma tumor cells to immunodeficient NOD.Cg-Prkdc^{scid}II2rg^{tm1Wjl}/SzJ (NSG) mice intraperitoneally injected with 4×10⁶ human peripheral blood mononuclear cells (PBMC).

FIG. 4A illustrates tumor burden over time in the mice treated with PBS relative to mice that received no tumor cells; FIG. 4B illustrates tumor burden over time in the mice treated with CD3-binding control bsAb (0.4 mg/kg)+4-1BB-binding control bsAb (4 mg/kg) relative to mice that received no tumor cells; FIG. 4C illustrates tumor burden over time in the mice treated with CD3-binding control bsAb (0.4 mg/kg)+CD38×4-1BB (4 mg/kg) relative to mice that received no tumor cells; FIG. 4D illustrates tumor burden over time in the mice treated with BCMA×CD3 bsAb (0.4 mg/kg)+4-1BB-binding control bsAb (4 mg/kg) relative to mice that received no tumor cells; FIG. 4E illustrates tumor burden over time in the mice treated with BCMA×CD3 bsAb (0.4 mg/kg)+CD38×4-1BB (4 mg/kg) relative to mice that received no tumor cells.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, the preferred methods and materials are now described. All patents, applications and non-patent publications mentioned in this specification are incorporated herein by reference in their entireties.

### Definitions

The expression “4-1BB”, as used herein, refers to a receptor for 4-1BBL. Crosslinking of 4-1BB by 4-1BBL enhances T cell activation. Human 4-1BB (or CD137) having UniProt accession number 007011 comprises the amino acid sequence as set forth in SEQ ID NO: 101; amino acid residues 1-23 are the signal peptide. Residues 24-186 make up the extracellular domain of the receptor.

Human 4-1BB (Immunogen) amino acid sequence
(SEQ ID NO: 101)
MGNSCYNIVATLLLVLNFERTRSLQDPCSNCPAGTFCDNNRNQICSPCP
PNSFSSAGGQRTCDICRQCKGVFRTRKECSSTSNAECDCTPGFHCLGAG
CSMCEQDCKQGQELTKKGCKDCCFGTFNDQKRGICRPWTNCSLDGKSVL
VNGTKERDVVCGPSPADLSPGASSVTPPAPAREPGHSPQIISFFLALTS
TALLFLLFFLTLRFSVVKRGRKKLLYIFKQPFMRPVQTTQEEDGCSCRF
PEEEEGGCEL

All references to proteins, polypeptides and protein fragments herein are intended to refer to the human version of the respective protein, polypeptide or protein fragment unless explicitly specified as being from a non-human species. Thus, the expression “4-1BB” means human 4-1BB unless specified as being from a non-human species, e.g., “mouse 4-1BB,” “monkey 4-1BB,” etc.

As used herein, “an antigen binding molecule that binds 4-1BB” or an “anti-4-1BB antigen binding molecule” includes antigen binding molecules that specifically recognize 4-1BB expressed on the surface of a cell. The anti-4-1BB antigen binding molecules provided herein comprise VRs and CDRs as disclosed herein. In certain embodiments, the antigen-binding molecules are antibodies. In certain embodiments, the antigen-binding molecules are bispecific antibodies.

The expression “CD38,” as used herein, also known as cyclic ADP ribose hydrolase, refers to a glycoprotein expressed on malignant plasma cells. CD38 plays a central role in regulating intracellular calcium levels. The protein has an N-terminal cytoplasmic tail, a single membrane-spanning domain, and a C-terminal extracellular region with four N-glycosylation sites. The term “CD38” as used herein, refers to the human CD38 protein unless specified as being from a non-human species (e.g., “mouse CD38”, “monkey CD38”, etc.). The human CD38 protein has the amino acid sequence shown in SEQ ID NO: 102 (Human CD38 extracellular domain (V43-I300).mFc), and/or having the amino acid sequence as set forth in NCBI accession No. NP_001766.2 or NM_001775.3.

Human CD38 extracellular domain (V43-1300).mFc
(Immunogen) amino acid
(SEQ ID NO: 102)
VPRWRQQWSGPGTTKRFPETVLARCVKYTEIHPEMRHVDCQSVWDAFKG
AFISKHPCNITEEDYQPLMKLGTQTVPCNKILLWSRIKDLAHQFTQVQR
DMFTLEDTLLGYLADDLTWCGEFNTSKINYQSCPDWRKDCSNNPVSVFW
KTVSRRFAEAACDVVHVMLNGSRSKIFDKNSTFGSVEVHNLQPEKVQTL
EAWVIHGGREDSRDLCQDPTIKELESIISKRNIQFSCKNIYRPDKFLQC
VKNPEDSSCTSEIEPRGPTIKPCPPCKCPAPNLLGGPSVFIFPPKIKDV
LMISLSPIVTCVVVDVSEDDPDVQISWFVNNVEVHTAQTQTHREDYNST
LRVVSALPIQHQDWMSGKEFKCKVNNKDLPAPIERTISKPKGSVRAPQV
YVLPPPEEEMTKKQVTLTCMVTDFMPEDIYVEWTNNGKTELNYKNTEPV
LDSDGSYFMYSKLRVEKKNWVERNSYSCSVVHEGLHNHHTTKSFSRTPG
K*
mFc sequence underlined

As used herein, “an antigen binding molecule that binds CD38” or an “anti-CD38 antigen binding molecule” includes antigen binding molecules that specifically recognize CD38.

The term “antigen binding molecule” includes multispecific antigen binding molecules, e.g., anti-CD38×anti-4-1BB 1+2 multispecific antigen binding molecules. The anti-CD38 antigen binding molecules provided herein comprise VRs and CDRs as disclosed herein. In certain embodiments, the antigen-binding molecules are antibodies. In certain embodiments, the antigen-binding molecules are bispecific antibodies.

The term “antigen binding molecule”, as used herein, means any antigen binding molecule or molecular complex comprising at least one complementarity determining region (CDR) that specifically binds to or interacts with a particular antigen (e.g., CD38 or 4-1BB). The term “antigen binding molecule” includes immunoglobulin molecules comprising four polypeptide chains, two heavy (H) chains and two light (L) chains inter-connected by disulfide bonds, as well as multimers thereof (e.g., IgM). The term “antigen binding molecule” includes immunoglobulin molecules comprising two antigen binding arms, A1 and A2. The term “antigen binding molecule” also includes immunoglobulin molecules consisting of four polypeptide chains, two heavy (H) chains and two light (L) chains inter-connected by disulfide bonds. Each antigen binding arm comprises a heavy chain, which in turn comprises at least one heavy chain variable region (abbreviated herein as HCVR or VH-1, VH-2, or VH-3) and a heavy chain constant region (CH1-1, CH1-2, and CH1-3). The heavy chain constant region also comprises CH2 and CH3. Each light chain comprises a light chain variable region (abbreviated herein as LCVR; on the A1 arm, VL-1; on the A2 arm, VL-2 and VL-3) and a light chain constant region (on the A1 arm, CL-1, and on the A2 arm, CL-2 and CL-3). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-CD38 antigen binding arm or anti-4-1BB antigen binding arm (or antigen binding portion thereof) may be identical to the human germline sequences, or may be naturally or artificially modified. An amino acid consensus sequence may be defined based on a side-by-side analysis of two or more CDRs.

The terms “antigen binding portion” of an antigen binding molecule, “antigen binding fragment thereof” of an antigen binding molecule, and the like, as used herein, include any naturally occurring, enzymatically obtainable, synthetic, or genetically engineered polypeptide or glycoprotein that specifically binds an antigen to form a complex. An antigen binding fragment of an antigen binding molecule may be derived, e.g., from full antigen binding molecule molecules using any suitable standard techniques such as proteolytic digestion or recombinant genetic engineering techniques involving the manipulation and expression of DNA encoding antigen binding molecule variable and optionally constant domains. Such DNA is known and/or is readily available from, e.g., commercial sources, DNA libraries (including, e.g., phage-antigen binding molecule libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

An anti-CD38×anti-4-1BB 1+2 antigen binding molecule will typically comprise at least three variable domains. A variable domain may be of any size or amino acid composition and will generally comprise at least one CDR which is adjacent to or in frame with one or more framework sequences. In having a VH domain associated with a VL domain, the VH and VL domains may be situated relative to one another in any suitable arrangement. For example, the variable region may be dimeric and contain VH-VH, VH-VL or VL-VL dimers. Alternatively, the antigen binding molecule may contain a monomeric VH or VL domain.

In certain embodiments, an antigen binding molecule may contain at least one variable domain covalently linked to at least one constant domain. Non-limiting, exemplary configurations of variable and constant domains that may be found within a multispecific antigen binding molecule of the present invention include: (i) V_{H}1-C_{H}1-1; (ii) V_{H}2-C_{H}1-2; (iii) V_{H}3-C_{H}1-3; (iv) V_{H}1-C_{H}1-C_{H}2; (V) V_{H}1-C_{H}1-C_{H}2-C_{H}3; (vi) V_{H}2-C_{H}1-2-C_{H}2; (vii) V_{H}2-C_{H}1-2-C_{H}2-C_{H}3; (viii) V_{H}3-C_{H}1-3; (ix) V_{H}3-C_{H}1-3-V_{H}2-C_{H}1-2; (x) V_{H}3-C_{H}1-3-V_{H}2-C_{H}1-2-C_{H}2; (xi) V_{H}3-C_{H}1-3-V_{H}2-C_{H}1-2-C_{H}2-C_{H}3; (xii) V_{H}-C_{L}; (xiii) V_{L}-1-C_{L}1; (xiv) V_{L}-2-C_{L}2; (xv) V_{L}-3-C_{L}3; (xvi) V_{L}-3-C_{L}3-C_{H}1-3; (xvii) V_{L}-2-C_{L}-2-C_{H}1-2; (xviii) V_{L}1-C_{L}1-C_{H}1-C_{H}2-C_{H}3; (xix) V_{L}-2-C_{L}2-C_{H}1-2; (xx) V_{L}-2-C_{L}2-C_{H}1-2- C_{H}2; (xxi) V_{L}-2-C_{L}2-C_{H}1-2-C_{H}2-C_{H}3; (xxii) VL-3-C_{L}3-C_{H}1-3-V_{H}2-C_{H}1-2-C_{H}2; (xxiii) VL-3-C_{L}3-C_{H}1-3-V_{H}2-C_{H}1-2-C_{H}2-C_{H}3; and (xxiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule.

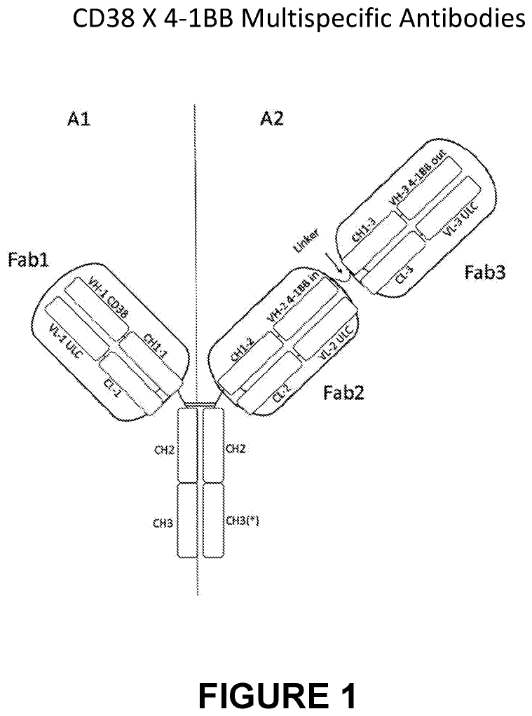

Illustratively, FIG. 1 shows an A1 antigen binding arm that comprises Fab1, which is specific for CD38, and an A2 antigen binding arm that comprises two Fabs, Fab2 and Fab3, each of which are specific to 4-1BB. The Fab1, Fab2, and Fab3 light chains can be universal light chains. In this example, the V_{L}-1 is linked to the C_{L}-1, which is linked to the C_{H}1-1 on the A1 arm. On the A2 arm, the V_{L}-3 is linked to the C_{L}-3, which is linked to the C_{H}1-3. Likewise, the V_{L}-2 is linked to the C_{L}-2, which is linked to the C_{H}1-2. The Fab2 and Fab3 of the anti-CD38×anti-4-1BB 1+2 construct are connected via a linker from the N-terminus of the V_{H}-2 4-1BB “IN” Fab2 to the C-terminus of the C_{H}1-3 “OUT” Fab3.

The antigen binding molecules of the present invention may function through complement-dependent cytotoxicity (CDC) or antigen binding molecule-dependent cell-mediated cytotoxicity (ADCC). “Complement-dependent cytotoxicity” (CDC) refers to lysis of antigen-expressing cells by an antigen binding molecule of the invention in the presence of complement. “Antigen binding molecule-dependent cell-mediated cytotoxicity” (ADCC) refers to a cell-mediated reaction in which nonspecific cytotoxic cells that express Fc receptors (FcRs) (e.g., Natural Killer (NK) cells, neutrophils, and macrophages) recognize bound antigen binding molecule on a target cell and thereby lead to lysis of the target cell. CDC and ADCC can be measured using assays that are well known and available in the art. (See, e.g., U.S. Pat. Nos. 5,500,362 and 5,821,337, and Clynes et al. (1998) Proc. Natl. Acad. Sci. (USA) 95:652-656). The constant region of an antigen binding molecule is important in the ability of an antigen binding molecule to fix complement and mediate cell-dependent cytotoxicity. Thus, the isotype of an antigen binding molecule may be selected on the basis of whether it is desirable for the antigen binding molecule to mediate cytotoxicity.

In certain embodiments of the invention, the anti-CD38×anti-4-1BB 1+2 multispecific antigen binding molecules provided herein are human antigen binding molecules. The term “human antigen binding molecule”, as used herein, is intended to include antigen binding molecules having variable and constant regions derived from human germline immunoglobulin sequences. The human antigen binding molecules of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antigen binding molecule”, as used herein, is not intended to include antigen binding molecules in which CDR sequences derived from the germline of another mammalian species, such as a mouse, have been grafted onto human framework sequences.

The multispecific antigen binding molecules provided herein may, in some embodiments, be recombinant human antigen binding molecules. The term “recombinant human antigen binding molecule”, as used herein, is intended to include all human antigen binding molecules that are prepared, expressed, created or isolated by recombinant means, such as antigen binding molecules expressed using a recombinant expression vector transfected into a host cell (described further below), antigen binding molecules isolated from a recombinant, combinatorial human antigen binding molecule library (described further below), antigen binding molecules isolated from an animal (e.g., a mouse) that is transgenic for human immunoglobulin genes (see e.g., Taylor et al. (1992) Nucl. Acids Res. 20:6287-6295) or antigen binding molecules prepared, expressed, created or isolated by any other means that involves splicing of human immunoglobulin gene sequences to other DNA sequences. Such recombinant human antigen binding molecules have variable and constant regions derived from human germline immunoglobulin sequences. In certain embodiments, however, such recombinant human antigen binding molecules are subjected to in vitro mutagenesis (or, when an animal transgenic for human Ig sequences is used, in vivo somatic mutagenesis) and thus the amino acid sequences of the V_{H} and V_{L} regions of the recombinant antigen binding molecules are sequences that, while derived from and related to human germline V_{H} and V_{L} sequences, may not naturally exist within the human antigen binding molecule germline repertoire in vivo.

Human antigen binding molecules can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antigen binding molecule). These forms have been extremely difficult to separate, even after affinity purification.

The frequency of appearance of the second form in various intact IgG isotypes is due to, but not limited to, structural differences associated with the hinge region isotype of the antigen binding molecule. A single amino acid substitution in the hinge region of the human IgG4 hinge can significantly reduce the appearance of the second form (Angal et al. (1993) Molecular Immunology 30:105) to levels typically observed using a human IgG1 hinge. The instant invention encompasses antigen binding molecules having one or more mutations in the hinge, C_{H}2 or C_{H}3 region which may be desirable, for example, in production, to improve the yield of the desired antigen binding molecule form.

The multispecific antigen binding molecules of the invention may be isolated antigen binding molecules. An “isolated multispecific antigen binding molecule,” as used herein, means an antigen binding molecule that has been identified and separated and/or recovered from at least one component of its natural environment. For example, an antigen binding molecule that has been separated or removed from at least one component of an organism, or from a tissue or cell in which the antigen binding molecule naturally exists or is naturally produced, is an “isolated antigen binding molecule” for purposes of the present invention. An isolated antigen binding molecule also includes an antigen binding molecule in situ within a recombinant cell. Isolated antigen binding molecules are antigen binding molecules that have been subjected to at least one purification or isolation step. According to certain embodiments, an isolated antigen binding molecule may be substantially free of other cellular material and/or chemicals.

The anti-CD38×anti-4-1BB 1+2 antigen binding molecules disclosed herein may comprise one or more amino acid substitutions, insertions and/or deletions in the framework and/or CDRs of the heavy and light chain variable domains as compared to the corresponding germline sequences from which the antigen binding molecules were derived. Such mutations can be readily ascertained by comparing the amino acid sequences disclosed herein to germline sequences available from, for example, public antigen binding molecule sequence databases. The present disclosure includes multispecific antigen binding molecules, or antigen binding fragments thereof, which are derived from any of the amino acid sequences disclosed herein, wherein one or more amino acids within one or more framework and/or CDRs are mutated relative to the sequences provided herein. In some aspects, the mutation is made to reflect the corresponding residue(s) of the germline sequence from which the antigen binding molecule was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antigen binding molecules and which comprise one or more mutations such as individual germline mutations or combinations thereof. In certain embodiments, all of the framework and/or CDR residues within the V_{H} and/or V_{L} domains are mutated back to the residues found in the original germline sequence from which the antigen binding molecule was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antigen binding molecule was originally derived).

Furthermore, the multispecific antigen binding molecules of the present invention may contain any combination of two or more mutations within the framework and/or CDR regions, e.g., wherein certain individual residues are mutated relative to the sequences provided herein, or are mutated to the corresponding residue of a particular germline sequence while certain other residues that differ from the original germline sequence are maintained or are mutated to the corresponding residue of a different germline sequence. Once obtained, antigen binding molecules and that contain one or more mutations can be easily tested for one or more desired property such as, improved binding specificity, increased binding affinity, improved or enhanced antagonistic or agonistic biological properties (as the case may be), reduced immunogenicity, etc. Antigen binding molecules and obtained in this general manner are encompassed within the present invention.

Provided herein are anti-CD38×anti-4-1BB antigen binding molecules comprising variants of any of the HCVR, LCVR, and/or CDR amino acid sequences disclosed herein having one or more substitutions. In some aspects, the substitutions are conservative amino acid substitutions. For example, the present disclosure includes anti-CD38×anti-4-1BB antigen binding molecules having HCVR, LCVR, and/or CDR amino acid sequences with, e.g., 10 or fewer, 8 or fewer, 6 or fewer, 4 or fewer, 3 or fewer, 2, or 1 amino acid substitutions relative to any of the HCVR, LCVR, and/or CDR amino acid sequences set forth in Tables 1, 3, 5, or 7 herein.

The term “epitope” refers to an antigenic determinant that interacts with a specific antigen binding site in the variable region of an antigen binding molecule known as a paratope. A single antigen may have more than one epitope. Thus, different antigen binding molecules may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Contemplated herein are amino acid substitutions that will not substantially change the functional properties of the multispecific antigen binding molecule. In some aspects, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Exemplary conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

### Binding Properties of the Antigen Binding Molecules

As used herein, the term “binding” in the context of the binding of an antigen binding molecule, immunoglobulin, antigen binding molecule-binding fragment, or Fc-containing protein to either, e.g., a predetermined antigen, such as a cell surface protein or fragment thereof, typically refers to an interaction or association between a minimum of two entities or molecular structures, such as an antigen binding molecule-antigen interaction.

For instance, binding affinity typically corresponds to a K_{D} value of about 10⁻⁷ M or less, such as about 10⁻⁸ M or less, such as about 10⁻⁹ M or less when determined by, for instance, surface plasmon resonance (SPR) technology in a BIAcore instrument using the antigen as the ligand and the antigen binding molecule, Ig, antigen binding molecule-binding fragment, or Fc-containing protein as the analyte (or antiligand). Cell-based binding strategies, such as fluorescent-activated cell sorting (FACS) binding assays, are also routinely used, and FACS data correlates well with other methods such as radioligand competition binding and SPR (Benedict, CA, J Immunol Methods. 1997, 201(2):223-31; Geuijen, C A, et al. J Immunol Methods. 2005, 302(1-2):68-77).

Accordingly, the multispecific antigen binding molecules or antigen binding protein of the invention binds to the predetermined antigen or cell surface molecule (receptor) having an affinity corresponding to a K_{D} value that is at least ten-fold lower than its affinity for binding to a non-specific antigen (e.g., BSA, casein). According to the present disclosure, the affinity of an antigen binding molecule corresponding to a K_{D} value that is equal to or less than ten-fold lower than a non-specific antigen may be considered non-detectable binding, however such an antigen binding molecule may be paired with a second antigen binding arm for the production of a multispecific antigen binding molecule of the invention.

The term “K_{D}” (M) refers to the dissociation equilibrium constant of a particular antigen binding molecule-antigen interaction, or the dissociation equilibrium constant of an antigen binding molecule or antigen binding molecule-binding fragment binding to an antigen. There is an inverse relationship between K_{D} and binding affinity, therefore the smaller the K_{D} value, the higher, i.e., stronger, the affinity. Thus, the terms “higher affinity” or “stronger affinity” relate to a higher ability to form an interaction and therefore a smaller K_{D} value, and conversely the terms “lower affinity” or “weaker affinity” relate to a lower ability to form an interaction and therefore a larger K_{D} value. In some circumstances, a higher binding affinity (or K_{D}) of a particular molecule (e.g. antigen binding molecule) to its interactive partner molecule (e.g. antigen X) compared to the binding affinity of the molecule (e.g. antigen binding molecule) to another interactive partner molecule (e.g. antigen Y) may be expressed as a binding ratio determined by dividing the larger K_{D} value (lower, or weaker, affinity) by the smaller K_{D} (higher, or stronger, affinity), for example expressed as 5-fold or 10-fold greater binding affinity, as the case may be.

The term “k_{d}” (sec-1 or 1/s) refers to the dissociation rate constant of a particular antigen binding molecule-antigen interaction, or the dissociation rate constant of an antigen binding molecule or antigen binding molecule-binding fragment. Said value is also referred to as the k_{off} value.

The term “kₐ” (M-1×sec-1 or 1/M/s) refers to the association rate constant of a particular antigen binding molecule-antigen interaction, or the association rate constant of an antigen binding molecule or antigen binding molecule-binding fragment.

The term “K_{A}” (M-1 or 1/M) refers to the association equilibrium constant of a particular antigen binding molecule-antigen interaction, or the association equilibrium constant of an antigen binding molecule or antigen binding molecule-binding fragment. The association equilibrium constant is obtained by dividing the kₐ by the k_{d}.

The term “EC50” or “EC₅₀” refers to the half maximal effective concentration, which includes the concentration of an antigen binding molecule which induces a response halfway between the baseline and maximum after a specified exposure time. The EC₅₀ essentially represents the concentration of an antigen binding molecule where 50% of its maximal effect is observed. In certain embodiments, the EC₅₀ value equals the concentration of an antigen binding molecule of the invention that gives half-maximal binding to cells expressing 4-1BB or tumor-associated antigen (e.g., CD38), as determined by e.g., a FACS binding assay. Thus, reduced or weaker binding is observed with an increased EC₅₀, or half maximal effective concentration value.

In one embodiment, decreased binding can be defined as an increased EC₅₀ antigen binding molecule concentration which enables binding to the half-maximal amount of target cells.

In another embodiment, the EC₅₀ value represents the concentration of an antigen binding molecule of the invention that elicits half-maximal depletion of target cells by T cell cytotoxic activity. Thus, increased cytotoxic activity (e.g., T cell-mediated tumor cell killing) is observed with a decreased EC₅₀, or half maximal effective concentration value.

### Multispecific Antigen Binding Molecules

The antigen binding molecules of the present invention bind both CD38 and 4-1BB.

Multispecific antigen binding molecules may be specific for different epitopes of one target polypeptide or may contain antigen binding domains specific for more than one target polypeptide. See, e.g., Tutt et al., 1991, J. Immunol. 147:60-69; Kufer et al., 2004, Trends Biotechnol. 22:238-244.

According to certain exemplary embodiments, the present invention includes multispecific antigen binding molecules that specifically bind 4-1BB and CD38. Such molecules may be referred to herein as, e.g., “anti-CD38×anti-4-1BB 1+2” or “anti-CD38/anti-4-1BB 1+2,” or “anti-CD38×4-1BB 1+2” or “CD38×4-1BB 1+2” multispecific molecules, or other similar terminology.

The present disclosure includes multispecific antigen binding molecules wherein one arm A2 of the immunoglobulin has two antigen-binding domains which bind human 4-1BB, a 4-1BB “IN” domain and a 4-1BB “OUT” domain, and the other arm A1 of the immunoglobulin is specific for binding human CD38. The 4-1BB-binding arm can comprise any of the HCVR/LCVR or CDR amino acid sequences as set forth in Table 3 (anti-4-1BB “IN”) or Table 5 (anti-4-1BB “OUT”) herein, in a stacked format.

In certain embodiments, the 4-1BB-binding arm binds to human 4-1BB and facilitates human T cell activation. In certain embodiments, the 4-1BB-binding arm binds to human 4-1BB and induces human T cell activation. In other embodiments, the 4-1BB-binding arm binds to human 4-1BB and induces tumor-associated antigen-expressing cell killing in the context of a multispecific or multispecific antigen binding molecule. The CD38-binding arm can comprise any of the HCVR/LCVR or CDR amino acid sequences as set forth in Table 1 herein.

As used herein, the expression “antigen binding molecule” means a protein, polypeptide or molecular complex comprising or consisting of at least one complementarity determining region (CDR) that alone, or in combination with one or more additional CDRs and/or framework regions (FRs), specifically binds to a particular antigen. In certain embodiments, an antigen binding molecule is an antigen binding molecule or a fragment of an antigen binding molecule, as those terms are defined elsewhere herein.

As used herein, the expression “multispecific antigen binding molecule” means a protein, polypeptide or molecular complex comprising at least a first antigen binding domain and a second antigen binding domain. Each antigen binding domain within the multispecific antigen binding molecule comprises at least one CDR that alone, or in combination with one or more additional CDRs and/or FRs, specifically binds to a particular antigen. In the context of the present invention, the first antigen binding arm A1 specifically binds a first antigen (e.g., CD38), and the second antigen binding arm A2 specifically binds a second and third antigen (e.g., 4-1BB), distinct from the first antigen.

The multispecific antigen binding molecules discussed herein can comprise a human IgG heavy chain constant region. In some cases, the human IgG heavy chain constant region is isotype IgG1. In some cases, the human IgG heavy chain constant region is isotype IgG4. In various embodiments, the multispecific antigen binding molecule comprises a chimeric hinge that reduces Fcγ receptor binding relative to a wild-type hinge of the same isotype.

The first antigen binding arm A1 and the second antigen binding arm A2 may be directly or indirectly connected to one another to form a multispecific antigen binding molecule of the present invention. Alternatively, the first antigen binding arm A1 and the second antigen binding arm A2 may each be connected to a separate multimerizing domain. The association of one multimerizing domain with another multimerizing domain facilitates the association between the two antigen binding domains, thereby forming a multispecific antigen binding molecule. As used herein, a “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin C_{H}3 domain. A non-limiting example of a multimerizing component is an Fc portion of an immunoglobulin (comprising a C_{H}2-C_{H}3 domain), e.g., an Fc domain of an IgG selected from the isotypes IgG1, IgG2, IgG3, and IgG4, as well as any allotype within each isotype group.

Multispecific antigen binding molecules of the present invention will typically comprise two multimerizing domains, e.g., two Fc domains that are each individually part of a separate antigen binding molecule heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In certain embodiments, the multimerizing domain is an Fc fragment or an amino acid sequence of from 1 to about 200 amino acids in length containing at least one cysteine residue. In other embodiments, the multimerizing domain is a cysteine residue, or a short cysteine-containing peptide. Other multimerizing domains include peptides or polypeptides comprising or consisting of a leucine zipper, a helix-loop motif, or a coiled-coil motif.

Any multispecific antigen binding molecule format or technology may be used to make the multispecific antigen binding molecules of the present invention. For example, an antigen binding molecule or fragment thereof having a first antigen binding specificity can be functionally linked (e.g., by chemical coupling, genetic fusion, noncovalent association or otherwise) to one or more other molecular entities, such as another antigen binding molecule or antigen binding molecule fragment having a second antigen binding specificity to produce a multispecific antigen binding molecule. Specific exemplary multispecific formats that can be used in the context of the present invention include, without limitation, e.g., scFv-based or diabody multispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab² multispecific formats (see, e.g., Klein et al. 2012, mAbs 4:6, 1-11, and references cited therein, for a review of the foregoing formats).

In the context of multispecific antigen binding molecules of the present invention, the multimerizing domains, e.g., Fc domains, may comprise one or more amino acid changes (e.g., insertions, deletions or substitutions) as compared to the wild-type, naturally occurring version of the Fc domain. For example, the invention includes multispecific antigen binding molecules comprising one or more modifications in the Fc domain that results in a modified Fc domain having a modified binding interaction (e.g., enhanced or diminished) between Fc and FcRn. In one embodiment, the multispecific antigen binding molecule comprises a modification in a C_{H}2 or a C_{H}3 region, wherein the modification increases the affinity of the Fc domain to FcRn in an acidic environment (e.g., in an endosome where pH ranges from about 5.5 to about 6.0). Non-limiting examples of such Fc modifications include, e.g., a modification at position 250 (e.g., E or Q); 250 and 428 (e.g., L or F); 252 (e.g., L/Y/F/W or T), 254 (e.g., or T), and 256 (e.g., S/R/Q/E/D or T); or a modification at position 428 and/or 433 (e.g., L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 2500 and 428L modification (e.g., T250Q and M428L); and a 307 and/or 308 modification (e.g., 308F or 308P).

The present disclosure also includes multispecific antigen binding molecules comprising a first C_{H}3 domain and a second Ig C_{H}3 domain, wherein the first and second Ig C_{H}3 domains differ from one another by at least one amino acid, and wherein at least one amino acid difference reduces binding of the multispecific antigen binding molecule to Protein A as compared to a bi-specific antigen binding molecule lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). The second C_{H}3 may further comprise a L105P modification (by IMGT; L455P by EU) See, for example, U.S. Pat. No. 8,586,713. Further modifications that may be found within the second C_{H}3 include: D16E, L18M, N44S, K52N, V57M, and V82I (by IMGT; D356E, L358M, N384S, K392N, V397M, and V4221 by EU) in the case of IgG1 antigen binding molecules; N44S, K52N, and V82I (IMGT; N384S, K392N, and V4221 by EU) in the case of IgG2 antigen binding molecules; and Q15R, N44S, K52N, V57M, R69K, E79Q, and V82I (by IMGT; Q355R, N384S, K392N, V397M, R409K, E419Q, and V4221 by EU) in the case of IgG4 antigen binding molecules.

In certain embodiments, the Fc domain may be chimeric, combining Fc sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antigen binding molecules set forth herein comprises, from N- to C-terminus: [IgG4 C_{H}1]-[IgG4 upper hinge]-[IgG2 lower hinge]-[IgG4 C_{H}2]-[IgG4 C_{H}3]. Another example of a chimeric Fc domain that can be included in any of the antigen binding molecules set forth herein comprises, from N- to C-terminus: [IgG1 C_{H}1]-[IgG1 upper hinge]-[IgG2 lower hinge]-[IgG4 C_{H}2]-[IgG1 C_{H}3]. These and other examples of chimeric Fc domains that can be included in any of the antigen binding molecules of the present invention are described in U.S. Pat. No. 9,359,437, which is herein incorporated in its entirety. Chimeric Fc domains having these general structural arrangements, and variants thereof, can have altered Fc receptor binding, which in turn affects Fc effector function.

### Sequence Variants

The antigen binding molecules and multispecific antigen binding molecules of the present invention may comprise one or more amino acid substitutions, insertions and/or deletions in the framework and/or CDR regions of the heavy and light chain variable domains as compared to the corresponding germline sequences from which the individual antigen binding domains were derived. Such mutations can be readily ascertained by comparing the amino acid sequences disclosed herein to germline sequences available from, for example, public antigen binding molecule sequence databases. The antigen binding molecules of the present invention may comprise antigen binding domains which are derived from any of the exemplary amino acid sequences disclosed herein, wherein one or more amino acids within one or more framework and/or CDR regions are mutated to the corresponding residue(s) of the germline sequence from which the antigen binding molecule was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antigen binding molecules and which comprise one or more individual germline mutations or combinations thereof. In certain embodiments, all of the framework and/or CDR residues within the V_{H} and/or V_{L} domains are mutated back to the residues found in the original germline sequence from which the antigen binding domain was originally derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antigen binding domain was originally derived).

Furthermore, the antigen binding domains may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antigen binding domains that contain one or more germline mutations can be easily tested for one or more desired property such as, improved binding specificity, increased binding affinity, improved or enhanced antagonistic or agonistic biological properties (as the case may be), reduced immunogenicity, etc. Multispecific antigen binding molecules comprising one or more antigen binding domains obtained in this general manner are encompassed within the present invention.

pH-Dependent Binding

The present invention includes anti-CD38×anti-4-1BB multispecific antigen binding molecules, with pH-dependent binding characteristics. For example, an anti-CD38 antigen binding arm of the present invention may exhibit reduced binding to CD38 at acidic pH as compared to neutral pH. Alternatively, anti-CD38 antigen binding arms of the invention may exhibit enhanced binding to CD38 at acidic pH as compared to neutral pH. The expression “acidic pH” includes pH values less than about 6.2, e.g., about 6.0, 5.95, 5, 9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding . . . at acidic pH as compared to neutral pH” is expressed in terms of a ratio of the K_{D} value of the antigen binding molecule binding to its antigen at acidic pH to the K_{D} value of the antigen binding molecule binding to its antigen at neutral pH (or vice versa). For example, an antigen binding molecule or may be regarded as exhibiting “reduced binding to CD38 at acidic pH as compared to neutral pH” for purposes of the present invention if the antigen binding molecule or exhibits an acidic/neutral K_{D} ratio of about 3.0 or greater. In certain exemplary embodiments, the acidic/neutral K_{D} ratio for an antigen binding molecule or of the present invention can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0 or greater.

Antigen binding molecules with pH-dependent binding characteristics may be obtained, e.g., by screening a population of antigen binding molecules for reduced (or enhanced) binding to a particular antigen at acidic pH as compared to neutral pH. Additionally, modifications of the antigen binding domain at the amino acid level may yield antigen binding molecules with pH-dependent characteristics. For example, by substituting one or more amino acids of an antigen binding domain (e.g., within a CDR) with a histidine residue, an antigen binding molecule with reduced antigen binding at acidic pH relative to neutral pH may be obtained.

### Antigen Binding Molecules Comprising Fc Variants

According to certain embodiments of the present invention, anti-CD38×anti-4-1BB multispecific antigen binding molecules, are provided comprising an Fc domain comprising one or more mutations which enhance or diminish antigen binding molecule binding to the FcRn receptor, e.g., at acidic pH as compared to neutral pH. For example, the present invention includes antigen binding molecules comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antigen binding molecule when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 2500 and 428L modification (e.g., T250Q and M428L); and a 307 and/or 308 modification (e.g., 308F or 308P).

For example, the present disclosure includes anti-CD38×anti-4-1BB 1+2 multispecific antigen binding molecules, comprising an Fc domain comprising one or more pairs or groups of mutations selected from the group consisting of: 2500 and 248L (e.g., T250Q and M248L); 252Y, 254T and 256E (e.g., M252Y, S254T and T256E); 428L and 434S (e.g., M428L and N434S); and 433K and 434F (e.g., H433K and N434F). All possible combinations of the foregoing Fc domain mutations, and other mutations within the antigen binding molecule variable domains disclosed herein, are contemplated within the scope of the present invention.

### Biological Characteristics of the Antigen Binding Molecules and Multispecific Antigen Binding Molecules

Provided herein are anti-CD38×anti-4-1BB multispecific antigen binding molecules that bind CD38 expressed on MOLP8 cells. As shown in Example 6, dose dependent binding of the CD38×4-1BB 1+2 (REGN7633, REGN7647 and REGN7650) and 1+1 (REGN7150) bispecific antibodies was observed in the presence of MOLP8 cells, with Max gMFI ranging from 8.8×10⁴ to 1.4×10⁵ and EC₅₀s ranging from 4.23×10⁻⁹ M to 9.27×10⁻⁹ M.

Provided herein are anti-CD38×anti-4-1BB multispecific antigen binding molecules that bind 4-1BB expressed on HEK293 cells engineered to express 4-1BB. Dose dependent binding of the CD38×4-1BB 1+2 (REGN7633, REGN7647 and REGN7650) and 1+1 (REGN7150) bispecific antibodies was observed in the presence of HEK293/h4-1BB cells, with Max gMFI ranging from 7.4×10⁵ to 2.4×10⁶ and EC₅₀s ranging from 1.47×10⁻⁸ M to 9.97×10⁻¹⁰ M.

CD38×4-1BB (1+1 and 1+2) multispecific antigen binding molecules mimic the natural ligand of 4-1BB by bridging CD38+ target cells with 4-1BB receptor positive T cells. In doing so, the constructs provide “signal 2” and enhance the activation of T cells in the presence of a “signal 1” provided by a Tumor-associated antigen (TAA)×CD3 bispecific antibody or an allogeneic response provided by the APC. As shown in Example 7, the multispecific antigen binding molecules provided herein, in the presence of target and “signal 1” (provided by REGN1979), led to higher maximum IL-2 response and greater potency than matched isotype controls in a T cell activation assay. As shown in Example 8, the multispecific antigen binding molecules had dose dependent increase in IL-2 and IFNγ and greater potency even when the linker length between the Fab2 and Fab3 was varied.

According to certain embodiments, multispecific antigen binding molecules provided herein activate 4-1BB receptor and stimulate 4-1BB activity in presence of target cells expressing CD38 as demonstrated in an engineered reporter assay. As shown in Example 9, 4-1BB activation was achieved using constructs with different linker lengths.

In certain embodiments, the multispecific antigen binding molecules provided herein cause dose dependent increases in IL-2 and IFNγ release. As shown in Example 10, in the presence of allogeneic NALM-6 cells or NALM-6 cells engineered to express PD-L1, CD38×4-1BB 1+2 antibody treatment (REGN7633, REGN7647 and REGN7650), led to dose dependent increases in IL-2 and IFNγ release and greater potency.

According to certain embodiments, treatment with the multispecific antigen-binding molecules when administered in combination with BCMA×CD3 bispecific antibodies, in vivo, results in a more potent, anti-tumor efficacy that is superior to either treatment alone. In Example 11, immunodeficient NOD.Cg-Prkdc^{scid}II2rg^{tm1Wjl}/SzJ (NSG) mice intraperitoneally injected with 4×10⁶ human peripheral blood mononuclear cells (PBMC) were administered human multiple myeloma cells. After receiving tumor cells, the mice were treated with CD3-binding control bispecific Ab or a BCMA×CD3 (REGN5458) bsAb at 0.4 mg/kg, in combination with a 4-1BB-binding control bispecific Ab (1+2 format) or a CD38×4-1BB (1+2 format; REGN9686) at 4 mg/kg. The treatment combinations were administered twice more on days 7 and 14, for a total of three doses. Combination treatment with BCMA×CD3 bsAb plus CD38×4-1BB 1+2 bsAb demonstrated more potent, combinatorial anti-tumor efficacy superior to either therapy alone.

### Epitope Mapping and Related Technologies

The epitope on CD38 and/or 4-1BB to which the antigen binding molecules of the present invention bind may consist of a single contiguous sequence of 3 or more (e.g., 3, 4, 5, 6, 7, 8, 9, 10, 11, 12, 13, 14, 15, 16, 17, 18, 19, 20 or more) amino acids of a respective CD38 or 4-1BB protein. Alternatively, the epitope may consist of a plurality of non-contiguous amino acids (or amino acid sequences) of CD38 or 4-1BB.

The term “epitope,” as used herein, refers to an antigenic determinant that interacts with a specific antigen binding site in the variable region of an antigen binding molecule p.m. known as a paratope. A single antigen may have more than one epitope. Thus, different antigen binding molecules may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstances, an epitope may include moieties of saccharides, phosphoryl groups, or sulfonyl groups on the antigen.

Various techniques known to persons of ordinary skill in the art can be used to determine whether an antigen binding domain of an antigen binding molecule “interacts with one or more amino acids” within a polypeptide or protein. Exemplary techniques include, e.g., routine cross-blocking assay such as that described in Antigen binding molecules, Harlow and Lane (Cold Spring Harbor Press, Cold Spring Harb., NY), alanine scanning mutational analysis, peptide blots analysis (Reineke, 2004, Methods Mol Biol 248:443-463), and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antigen binding domain of an antigen binding molecule interacts is hydrogen/deuterium exchange detected by mass spectrometry. In general terms, the hydrogen/deuterium exchange method involves deuterium-labeling the protein of interest, followed by binding the antigen binding molecule to the deuterium-labeled protein. Next, the protein/antigen binding molecule complex is transferred to water to allow hydrogen-deuterium exchange to occur at all residues except for the residues protected by the antigen binding molecule (which remain deuterium-labeled). After dissociation of the antigen binding molecule, the target protein is subjected to protease cleavage and mass spectrometry analysis, thereby revealing the deuterium-labeled residues which correspond to the specific amino acids with which the antigen binding molecule interacts. See, e.g., Ehring (1999) Analytical Biochemistry 267(2):252-259; Engen and Smith (2001) Anal. Chem. 73:256A-265A. X-ray crystallography of the antigen/antigen binding molecule complex may also be used for epitope mapping purposes.

Provided herein are anti-CD38 antigen binding arms A1 that bind to the same epitope as any of the specific exemplary antigen binding arms described herein (e.g., antigen binding molecules comprising any of the amino acid sequences as set forth in Table 1 herein). Likewise, the present invention also includes anti-CD38 antigen binding arms A1 that compete for binding to CD38 with any of the specific exemplary antigen binding arms described herein (e.g., antigen binding molecules comprising any of the amino acid sequences as set forth in Table 1 herein).

Provided herein are anti-4-1BB antigen binding arms A2 comprising a first antigen-binding domain (R1) and a second antigen-binding domain (R2), where either R1 or R2 bind to the same epitope as any of the specific exemplary antigen binding domains described herein (e.g., antigen binding arms comprising any of the amino acid sequences as set forth in Table 3 or Table 5 herein). Likewise, the present invention also includes anti-4-1BB antigen binding molecules that compete for binding to 4-1BB with any of the specific exemplary antigen binding domains described herein (e.g., antigen binding arms comprising any of the amino acid sequences as set forth in Table 3 or Table 5 herein).

Likewise, provided herein are multispecific antigen binding molecules comprising a first antigen binding arm that specifically binds human CD38 (Fab1), and a second antigen binding arm that specifically binds human 4-1BB (Fab 2 and Fab 3), wherein the first antigen binding domain competes for binding to CD38 with any of the specific exemplary CD38-specific antigen binding arms described herein, and/or wherein the second antigen binding arm competes for binding to 4-1BB with any of the specific exemplary 4-1BB-specific antigen binding Fabs described herein.

One can easily determine whether a particular antigen binding molecule (e.g., multispecific 1+2 antigen binding molecule) or antigen binding fragment thereof binds to the same epitope as, or competes for binding with, a reference antigen binding molecule of the present invention by using routine methods known in the art. For example, to determine if a test antigen binding molecule binds to the same epitope on CD38 (or 4-1BB) as a reference multispecific antigen binding molecule of the present invention, the reference multispecific molecule is first allowed to bind to a CD38 protein (or 4-1BB protein). Next, the ability of a test antigen binding molecule to bind to the CD38 (or 4-1BB) molecule is assessed. If the test antigen binding molecule is able to bind to CD38 (or 4-1BB) following saturation binding with the reference multispecific antigen binding molecule, it can be concluded that the test antigen binding molecule binds to a different epitope of CD38 (or 4-1BB) than the reference multispecific antigen binding molecule. On the other hand, if the test antigen binding molecule is not able to bind to the CD38 (or 4-1BB) molecule following saturation binding with the reference multispecific antigen binding molecule, then the test antigen binding molecule may bind to the same epitope of CD38 (or 4-1BB) as the epitope bound by the reference multispecific antigen binding molecule of the invention. Additional routine experimentation (e.g., peptide mutation and binding analyses) can then be carried out to confirm whether the observed lack of binding of the test antigen binding molecule is in fact due to binding to the same epitope as the reference multispecific antigen binding molecule or if steric blocking (or another phenomenon) is responsible for the lack of observed binding. Experiments of this sort can be performed using ELISA, RIA, Biacore, flow cytometry or any other quantitative or qualitative antigen binding molecule-binding assay available in the art. In accordance with certain embodiments of the present invention, two antigen binding proteins bind to the same (or overlapping) epitope if, e.g., a 1-, 5-, 10-, 20- or 100-fold excess of one antigen binding protein inhibits binding of the other by at least 50% but preferably 75%, 90% or even 99% as measured in a competitive binding assay (see, e.g., Junghans et al., Cancer Res. 1990:50:1495-1502). Alternatively, two antigen binding proteins are deemed to bind to the same epitope if essentially all amino acid mutations in the antigen that reduce or eliminate binding of one antigen binding protein reduce or eliminate binding of the other. Two antigen binding proteins are deemed to have “overlapping epitopes” if only a subset of the amino acid mutations that reduce or eliminate binding of one antigen binding protein reduce or eliminate binding of the other.

To determine if an antigen binding molecule or antigen binding domain thereof competes for binding with a reference antigen binding molecule, the above-described binding methodology is performed in two orientations: In a first orientation, the reference antigen binding molecule is allowed to bind to a CD38 protein (or 4-1BB protein) under saturating conditions followed by assessment of binding of the test antigen binding molecule to the CD38 (or 4-1BB) molecule. In a second orientation, the test antigen binding molecule is allowed to bind to a CD38 (or 4-1BB) molecule under saturating conditions followed by assessment of binding of the reference antigen binding molecule to the CD38 (or 4-1BB) molecule. If, in both orientations, only the first (saturating) antigen binding molecule is capable of binding to the CD38 (or 4-1BB) molecule, then it is concluded that the test antigen binding molecule and the reference antigen binding molecule compete for binding to CD38 (or 4-1BB). As will be appreciated by a person of ordinary skill in the art, an antigen binding molecule that competes for binding with a reference antigen binding molecule may not necessarily bind to the same epitope as the reference antigen binding molecule, but may sterically block binding of the reference antigen binding molecule by binding an overlapping or adjacent epitope.

### Preparation of Antigen Binding Domains and Construction of Multispecific Molecules

Antigen binding domains specific for particular antigens can be prepared by any antigen binding molecule generating technology known in the art. Once obtained, different antigen binding domains provided herein, specific for two different antigens (e.g., CD38 and 4-1BB), can be appropriately arranged relative to one another to produce a multispecific antigen binding molecule of the present invention using routine methods. (A discussion of exemplary multispecific antigen binding molecule formats that can be used to construct the multispecific antigen binding molecules of the present invention is provided elsewhere herein). In certain embodiments, one or more of the individual components (e.g., heavy and light chains) of the multispecific antigen binding molecules of the invention are derived from chimeric, humanized or fully human antigen binding molecules. Methods for making such antigen binding molecules are well known in the art. For example, one or more of the heavy and/or light chains of the multispecific antigen binding molecules of the present invention can be prepared using VELOCIMMUNE™ technology. Using VELOCIMMUNE™ technology (or any other human antigen binding molecule generating technology), high affinity chimeric antigen binding molecules to a particular antigen (e.g., CD38 or 4-1BB) are initially isolated having a human variable region and a mouse constant region. The antigen binding molecules are characterized and selected for desirable characteristics, including affinity, selectivity, epitope, etc. The mouse constant regions are replaced with a desired human constant region to generate fully human heavy and/or light chains that can be incorporated into the multispecific antigen binding molecules of the present invention.

Genetically engineered animals may be used to make human multispecific antigen binding molecules. For example, a genetically modified mouse can be used which is incapable of rearranging and expressing an endogenous mouse immunoglobulin light chain variable sequence, wherein the mouse expresses only one or two human light chain variable domains encoded by human immunoglobulin sequences operably linked to the mouse kappa constant gene at the endogenous mouse kappa locus. Such genetically modified mice can be used to produce fully human antigen binding molecules comprising two different heavy chains that associate with an identical light chain that comprises a variable domain derived from one of two different human light chain variable region gene segments. (See, e.g., US 2011/0195454). Fully human refers to an antigen binding molecule, or antigen binding fragment thereof, or immunoglobulin domain thereof, comprising an amino acid sequence encoded by a DNA derived from a human sequence over the entire length of each polypeptide of the antigen binding molecule or antigen binding fragment thereof, or immunoglobulin domain thereof. In some instances, the fully human sequence is derived from a protein endogenous to a human. In other instances, the fully human protein or protein sequence comprises a chimeric sequence wherein each component sequence is derived from human sequence. While not being bound by any one theory, chimeric proteins or chimeric sequences are generally designed to minimize the creation of immunogenic epitopes in the junctions of component sequences, e.g., compared to any wild-type human immunoglobulin regions or domains.

### Bioequivalents

Provided herein are antigen binding molecules having amino acid sequences that vary from those of the exemplary molecules disclosed herein but that retain the ability to bind CD38 and/or 4-1BB. Such variant molecules may comprise one or more additions, deletions, or substitutions of amino acids when compared to parent sequence, but exhibit biological activity that is essentially equivalent to that of the described multispecific antigen binding molecules.

Antigen binding molecules that are bioequivalent to any of the exemplary antigen binding molecules set forth herein are pharmaceutical equivalents or pharmaceutical alternatives whose rate and extent of absorption do not show a significant difference when administered at the same molar dose under similar experimental conditions, either single dose or multiple doses. Some antigen binding proteins will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen binding proteins are bioequivalent if there are no clinically meaningful differences in their safety, purity, and potency.

In one embodiment, two antigen binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antigen binding molecule or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antigen binding molecule (or its target) is measured as a function of time; and (d) in a well-controlled clinical trial that establishes safety, efficacy, or bioavailability or bioequivalence of an antigen binding protein.

Bioequivalent variants of the exemplary multispecific antigen binding molecules set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antigen binding proteins may include variants of the exemplary multispecific antigen binding molecules set forth herein comprising amino acid changes which modify the glycosylation characteristics of the molecules, e.g., mutations which eliminate or remove glycosylation.

### Species Selectivity and Species Cross-Reactivity

According to certain embodiments of the invention, antigen binding molecules are provided which bind to human 4-1BB but not to 4-1BB from other species. Also provided are antigen binding molecules which bind to human CD38, but not to CD38 from other species. The present invention also includes antigen binding molecules that bind to human 4-1BB and to CD38 from one or more non-human species; and/or antigen binding molecules that bind to human 4-1BB and to 4-1BB from one or more non-human species.

According to certain exemplary embodiments of the invention, antigen binding molecules are provided which bind to human CD38 and/or human 4-1BB and may bind or not bind, as the case may be, to one or more of mouse, rat, guinea pig, hamster, gerbil, pig, cat, dog, rabbit, goat, sheep, cow, horse, camel, cynomolgus, marmoset, rhesus or chimpanzee CD38 and/or 4-1BB. For example, in particular exemplary embodiments of the disclosed herein, multispecific antigen binding molecules are provided comprising a first antigen binding arm that binds human CD38 or cynomolgus CD38, and a second antigen binding arm comprising a first antigen-binding domain and a second antigen-binding domain wherein the second antigen-binding arm specifically binds human 4-1BB, or multispecific antigen binding molecules comprising a second antigen binding arm comprising a first and second antigen-binding domains that bind human 4-1BB and/or cynomolgus 4-1BB, and a first antigen binding arm that specifically binds human CD38.

### Therapeutic Formulation and Administration

The present invention provides pharmaceutical compositions comprising the multispecific antigen binding molecules disclosed herein. The pharmaceutical compositions of the invention are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of multispecific antigen binding molecule administered to a patient may vary depending upon the age and the size of the patient, target disease, conditions, route of administration, and the like. The preferred dose is typically calculated according to body weight or body surface area. When a multispecific antigen binding molecule of the present invention is used for therapeutic purposes in an adult patient, it may be advantageous to intravenously administer the multispecific antigen binding molecule of the present invention normally at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted. Effective dosages and schedules for administering a multispecific antigen binding molecule may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition of the present invention can be delivered subcutaneously, intramuscularly, or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park IL), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antigen binding molecule or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antigen binding molecule contained is generally about 1 to about 1000 mg per dosage form in a unit dose; especially in the form of injection, it is preferred that the aforesaid antigen binding molecule is contained in about 1 to about 100 mg and in about 10 to about 250 mg for the other dosage forms. In some embodiments, the unit dosage can be as much as about 750 mg, 800 mg, 900 mg, or 1000 mg.

### Therapeutic Uses of the Antigen Binding Molecules

The present invention includes methods comprising administering to a subject in need thereof a therapeutic composition a multispecific antigen binding molecule that specifically binds CD38 and 4-1BB. The therapeutic composition can comprise any of the multispecific antigen binding molecules as disclosed herein and a pharmaceutically acceptable carrier or diluent. As used herein, the expression “a subject in need thereof” means a human or non-human animal that exhibits one or more symptoms or indicia of cancer (e.g., a subject expressing a tumor or suffering from any of the cancers mentioned herein below), or who otherwise would benefit from an inhibition or reduction in CD38 activity or a depletion of CD38+ cells (e.g., multiple myeloma cells).

The multispecific antigen binding molecules of the invention (and therapeutic compositions comprising the same) are useful, inter alia, for treating any disease or disorder in which stimulation, activation and/or targeting of an immune response would be beneficial. In particular, the anti-CD38×anti-4-1BB 1+2 multispecific antigen binding molecules of the present invention may be used for the treatment, prevention and/or amelioration of any disease or disorder associated with or mediated by CD38 and/or BCMA expression or activity or the proliferation of CD38+ and/or BCMA+ cells. The mechanism of action by which the therapeutic methods of the invention are achieved include killing of the cells expressing CD38 in the presence of effector cells, for example, by CDC, apoptosis, ADCC, phagocytosis, or by a combination of two or more of these mechanisms. Cells expressing CD38 which can be inhibited or killed using the multispecific antigen binding molecules of the invention include, for example, multiple myeloma cells.

The multispecific antigen binding molecules of the present disclosure may be used to treat a disease or disorder associated with CD38 expression including, e.g., multiple myeloma, B-cell leukemia, hepatocellular carcinoma, non-small cell lung cancer, melanoma, pancreatic ductal adenocarcinoma, glioma, or breast cancer, or another cancer characterized in part by having CD38+ cells.

According to certain embodiments, the anti-CD38×anti-4-1BB 1+2 antigen binding molecules are useful for inhibiting growth of a plasma cell tumor in a subject. In some aspects, the plasma cell tumor is multiple myeloma.

The multispecific antigen binding molecules of the present disclosure may be used to inhibit growth of a tumor in a subject. The tumor is selected from the group consisting of multiple myeloma, lymphoma, B-cell leukemia, hepatocellular carcinoma, non-small cell lung cancer, melanoma, pancreatic ductal adenocarcinoma, glioma, or breast cancer, or another cancer characterized in part by having CD38+ cells.

According to certain embodiments, the anti-CD38×anti-4-1BB 1+2 antigen binding molecules are useful for treating tumor cells expressing, for example, BCMA or CD20. The antigen binding molecules provided herein may also be used to treat a disease or disorder associated with BCMA expression including, e.g., a cancer including multiple myeloma or other B-cell or plasma cell cancers, such as Waldenström's macroglobulinemia, Burkitt lymphoma, and diffuse large B-Cell lymphoma, Non-Hodgkin's lymphoma, chronic lymphocytic leukemia, follicular lymphoma, mantle cell lymphoma, marginal zone lymphoma, lymphoplasmacytic lymphoma, and Hodgkin's lymphoma. According to certain embodiments of the present invention, the anti-CD38×anti-4-1BB antigen binding molecules are useful for treating a patient afflicted with multiple myeloma. According to other related embodiments of the invention, methods are provided comprising administering an anti-CD38×anti-4-1BB multispecific antigen binding molecule provided herein in combination with an anti-BCMA antigen binding molecule, or an anti-BCMA×anti-CD3 multispecific antigen binding molecule, or an anti-CD20×anti-CD3 multispecific antigen binding molecule, or an anti-CD28×anti-4-1BB multispecific antigen binding molecule as disclosed herein to a patient who is afflicted with cancer cells expressing BCMA or CD20. Analytic/diagnostic methods known in the art, such as tumor scanning, etc., may be used to ascertain whether a patient harbors multiple myeloma or another B-cell lineage cancer.

In some embodiments, an anti-CD38×anti-4-1BB multispecific antigen binding molecule provided herein can be administered in combination with second therapeutic agent or therapeutic regimen comprising a chemotherapeutic drug, DNA alkylators, immunomodulators, proteasome inhibitors, histone deacetylase inhibitors, radiotherapy, a stem cell transplant, a different bispecific antibody that interacts with a different tumor cell surface antigen and a T cell or immune cell antigen, an antibody drug conjugate, a bispecific antibody conjugated to an anti-tumor agent, a PD-1 inhibitor (such as an anti-PD-1 antibody, e.g., cemiplimab), a PD-L1 inhibitor, a CTLA-4 checkpoint inhibitor, or combinations thereof.

The present invention also includes methods for treating residual cancer in a subject. As used herein, the term “residual cancer” means the existence or persistence of one or more cancerous cells in a subject following treatment with an anti-cancer therapy.

According to certain aspects, the present invention provides methods for treating a disease or disorder associated with CD38 expression (e.g., multiple myeloma) comprising administering one or more of the anti-CD38×anti-4-1BB 1+2 antigen binding molecules described herein to a subject after the subject has been determined to have multiple myeloma. For example, the present invention includes methods for treating multiple myeloma comprising administering an anti-CD38×anti-4-1BB multispecific antigen binding molecule to a patient 1 day, 2 days, 3 days, 4 days, 5 days, 6 days, 1 week, 2 weeks, 3 weeks or 4 weeks, 2 months, 4 months, 6 months, 8 months, 1 year, or more after the subject has received other immunotherapy or chemotherapy.

### Combination Therapies and Formulations

The present invention provides methods which comprise administering a pharmaceutical composition comprising any of the exemplary antigen binding molecules and multispecific antigen binding molecules described herein in combination with one or more additional therapeutic agents. Exemplary additional therapeutic agents that may be therapeutically combined with or administered in combination with an antigen binding molecule of the present invention include, e.g., an anti-tumor agent (e.g., chemotherapeutic agents including melphalan, vincristine (Oncovin), cyclophosphamide (Cytoxan), etoposide (VP-16), doxorubicin (Adriamycin), liposomal doxorubicin (Doxil), obendamustine (Treanda), or any others known to be effective in treating a plasma cell tumor in a subject). In some embodiments, the second therapeutic agent comprises steroids. In some embodiments, the second therapeutic agent comprises targeted therapies including thalidomide, lenalidomide, and bortezomib, which are therapies approved to treat newly diagnosed patients. Lenalidomide, pomalidomide, bortezomib, carfilzomib, panobinostat, ixazomib, elotuzumab, and daratumumab are examples of a second therapeutic agent effective for treating recurrent myeloma.

In some embodiments, the second therapeutic is an anti-BCMA×CD3 bispecific antigen binding molecule. Illustrative anti-BCMA×CD3 bispecific antigen binding molecules are disclosed in U.S. 2020/0024356 incorporated by reference herein. An exemplary anti-BCMA×CD3 bispecific antigen binding molecule, as disclosed in U.S. 2020/0024356, is REGN5458. In some embodiments, the second therapeutic is an anti-CD20×CD3 bispecific antigen binding molecule. Illustrative anti-CD20×CD3 bispecific antigen binding molecules are disclosed in U.S. Pat. No. 9,657,102, incorporated by reference herein. An exemplary anti-CD20×CD3 bispecific antigen binding molecule is REGN1979 (U.S. Pat. No. 9,657,102).

In certain embodiments the second therapeutic agent is a regimen comprising radiotherapy or a stem cell transplant. In certain embodiments, the second therapeutic agent may be an immunomodulatory agent. In certain embodiments, the second therapeutic agent may be a proteasome inhibitor, including bortezomib (Velcade), carfilzomib (Kyprolis), ixazomib (Ninlaro). In certain embodiments the second therapeutic agent may be a histone deacetylase inhibitor such as panobinostat (Farydak). In certain embodiments, the second therapeutic agent may be a monoclonal antibody, an antibody drug conjugate, a multispecific/bispecific/monospecific antigen binding molecule conjugated to an anti-tumor agent, a checkpoint inhibitor, an oncolytic virus, a cancer vaccine, a CAR-T cell, or combinations thereof. Other agents that may be beneficially administered in combination with the antigen binding molecules of the invention include cytokine inhibitors, including small-molecule cytokine inhibitors and antigen binding molecules that bind to cytokines such as IL-1, IL-2, IL-3, IL-4, IL-5, IL-6, IL-8, IL-9, IL-11, IL-12, IL-13, IL-17, IL-18, or to their respective receptors. The pharmaceutical compositions of the present invention (e.g., pharmaceutical compositions comprising an anti-CD38×anti-4-1BB multispecific antigen binding molecule as disclosed herein) may also be administered as part of a therapeutic regimen comprising one or more therapeutic combinations selected from a monoclonal antigen binding molecule other than those described herein, which may interact with a different antigen on the plasma cell surface, a multispecific antigen binding molecule, which has one arm that binds to an antigen on the tumor cell surface and the other arm binds to an antigen on a T cell, an antibody drug conjugate, a bispecific antibody conjugated with an anti-tumor agent, a checkpoint inhibitor, for example, one that targets, PD-1 or CTLA-4, or combinations thereof. In certain embodiments, the checkpoint inhibitors may be selected from PD-1 inhibitors, such as pembrolizumab (Keytruda), nivolumab (Opdivo), or cemiplimab (REGN2810; Libtayo). In certain embodiments, the checkpoint inhibitors may be selected from PD-L1 inhibitors, such as atezolizumab (Tecentriq), avelumab (Bavencio), or Durvalumab (Imfinzi)). In certain embodiments, the checkpoint inhibitors may be selected from CTLA-4 inhibitors, such as ipilimumab (Yervoy). Other combinations that may be used in conjunction with an antigen binding molecule of the invention are described above.

The present invention also includes therapeutic combinations comprising any of the antigen binding molecules mentioned herein and an inhibitor of one or more of VEGF, Ang2, DLL4, EGFR, ErbB2, ErbB3, ErbB4, EGFRvIII, cMet, IGF1R, B-raf, PDGFR-α, PDGFR-β, FOLH1 (PSMA), PRLR, STEAP1, STEAP2, TMPRSS2, MSLN, CA9, uroplakin, or any of the aforementioned cytokines, wherein the inhibitor is an aptamer, an antisense molecule, a ribozyme, an siRNA, a peptibody, a nanobody or an antigen binding molecule fragment (e.g., Fab fragment; F(ab′)₂ fragment; Fd fragment; Fv fragment; scFv; dAb fragment; or other engineered molecules, such as diabodies, triabodies, tetrabodies, minibodies and minimal recognition units). The antigen binding molecules of the invention may also be administered and/or co-formulated in combination with antivirals, antibiotics, analgesics, corticosteroids and/or NSAIDs. The antigen binding molecules of the invention may also be administered as part of a treatment regimen that also includes radiation treatment and/or conventional chemotherapy.

The additional therapeutically active component(s) may be administered just prior to, concurrent with, or shortly after the administration of an antigen binding molecule of the present invention; (for purposes of the present disclosure, such administration regimens are considered the administration of an antigen binding molecule “in combination with” an additional therapeutically active component).

The present invention includes pharmaceutical compositions in which an antigen binding molecule of the present invention is co-formulated with one or more of the additional therapeutically active component(s) as described elsewhere herein.

### Administration Regimens

According to certain embodiments of the present invention, multiple doses of an antigen binding molecule (e.g., a multispecific antigen binding molecule that specifically binds CD38 and 4-1BB) may be administered to a subject over a defined time course. The methods according to this aspect of the invention comprise sequentially administering to a subject multiple doses of a multispecific antigen binding molecule of the invention. As used herein, “sequentially administering” means that each dose of an antigen binding molecule is administered to the subject at a different point in time, e.g., on different days separated by a predetermined interval (e.g., hours, days, weeks or months). The present invention includes methods which comprise sequentially administering to the patient a single initial dose of an antigen binding molecule, followed by one or more secondary doses of the antigen binding molecule, and optionally followed by one or more tertiary doses of the antigen binding molecule.

The terms “initial dose,” “secondary doses,” and “tertiary doses,” refer to the temporal sequence of administration of the antigen binding molecule of the invention. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the antigen binding molecule, but generally may differ from one another in terms of frequency of administration. In certain embodiments, however, the amount of an antigen binding molecule contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In one exemplary embodiment of the present invention, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of antigen binding molecule which is administered to a patient prior to the administration of the very next dose in the sequence with no intervening doses.

The methods according to this aspect of the invention may comprise administering to a patient any number of secondary and/or tertiary doses of an antigen binding molecule (e.g., a multispecific 1+2 antigen binding molecule that specifically binds CD38 and 4-1BB). For example, in certain embodiments, only a single secondary dose is administered to the patient.

In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

### Diagnostic Uses of the Antigen Binding Molecules

The anti-CD38 antigen binding molecules disclosed herein may be used to detect and/or measure CD38, or CD38-expressing cells in a sample, e.g., for diagnostic purposes. For example, an anti-CD38 antigen binding molecule, or fragment thereof, may be used to diagnose a condition or disease characterized by aberrant expression (e.g., over-expression, under-expression, lack of expression, etc.) of CD38. Exemplary diagnostic assays for CD38 may comprise, e.g., contacting a sample, obtained from a patient, with an anti-CD38 antigen binding molecule disclosed herein, wherein the anti-CD38 antigen binding molecule is labeled with a detectable label or reporter molecule. Alternatively, an unlabeled anti-CD38 antigen binding molecule can be used in diagnostic applications in combination with a secondary antigen binding molecule which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, beta-galactosidase, horseradish peroxidase, or luciferase. Another exemplary diagnostic use of the anti-CD38 antigen binding molecules of the invention includes ⁸⁹Zr-labeled, such as ⁸⁹Zr-desferrioxamine-labeled, antigen binding molecules for the purpose of noninvasive identification and tracking of tumor cells in a subject (e.g., positron emission tomography (PET) imaging). (See, e.g., Tavare, R. et al. Cancer Res. 2016 Jan. 1; 76(1):73-82; and Azad, B B. et al. Oncotarget. 2016 Mar. 15; 7(11):12344-58.) Specific exemplary assays that can be used to detect or measure CD38 in a sample include enzyme-linked immunosorbent assay (ELISA), radioimmunoassay (RIA), and fluorescence-activated cell sorting (FACS).

Samples that can be used in CD38 diagnostic assays according to the present invention include any tissue or fluid sample obtainable from a patient which contains detectable quantities of CD38 protein, or fragments thereof, under normal or pathological conditions. Generally, levels of CD38 in a particular sample obtained from a healthy patient (e.g., a patient not afflicted with a disease or condition associated with abnormal CD38 levels or activity) will be measured to initially establish a baseline, or standard, level of CD38. This baseline level of CD38 can then be compared against the levels of CD38 measured in samples obtained from individuals suspected of having a CD38 related disease (e.g., a tumor containing CD38-expressing cells) or condition.

### Devices

The present invention also provides a vessel (e.g., a vial or chromatography column) or injection device (e.g., syringe, pre-filled syringe or autoinjector) comprising a multispecific antigen binding molecule (e.g., pharmaceutical formulation thereof) set forth herein. The vessel or injection device may be packaged into a kit.

An injection device is a device that introduces a substance into the body of a subject (e.g., a human) via a parenteral route, e.g., intraocular, intravitreal, intramuscular, subcutaneous or intravenous. For example, an injection device may be a syringe (e.g., pre-filled with the pharmaceutical formulation, such as an auto-injector) which, for example, includes a cylinder or barrel for holding fluid to be injected (e.g., comprising the antigen binding molecule or fragment or a pharmaceutical formulation thereof), a needle for piecing skin, blood vessels or other tissue for injection of the fluid; and a plunger for pushing the fluid out of the cylinder and through the needle bore and into the body of the subject.

A pharmaceutical composition provided herein can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L. P.), and the HUMIRA™ Pen (Abbott Labs, Abbott Park Ill.), to name only a few.

Provided herein are methods for administering a multispecific antigen binding molecule of the present disclosure comprising introducing e.g., injecting, the molecule into the body of the subject, e.g., with an injection device.

### Expression Methods

Provided herein are recombinant methods for making a multispecific antigen binding molecule of the present invention, or an immunoglobulin chain thereof, comprising (i) introducing, into a host cell, one or more polynucleotides encoding light and/or heavy immunoglobulin chains of such a multispecific antigen binding molecule, for example, wherein the one or more polynucleotides is comprised in one or more vectors; and/or integrates into the host cell chromosome and/or is operably linked to a promoter; (ii) culturing the host cell (e.g., mammalian, fungal, Chinese hamster ovary (CHO), Pichia or Pichia pastoris) under conditions favorable to expression of the polynucleotide and, (iii) optionally, isolating the multispecific antigen binding molecule or immunoglobulin chain from the host cell and/or medium in which the host cell is grown. The product of such a method also forms part of the present disclosure along with a pharmaceutical composition thereof.

In some aspects, step (i) comprises cloning the individual A1 heavy chain, the A2 heavy chain, and the universal light chain into separate expression vectors. For example, CD38-binding heavy chain variable regions (HCVR) (VH-1) can be cloned into a heavy chain expression plasmid (CH1-1_CH2_CH3). 4-1BB-binding heavy chain variable regions (HCVR) (VH-3) fused to a CH1 domain (CH1-3) with linkers of various length (linker) followed by another 4-1BB-binding heavy chain variable regions (HCVR) (VH-2) can be cloned into a heavy chain expression plasmid (CH1-2_CH2_CH3(*)) containing the mutations H435R, and Y436F, (EU numbering) (U.S. Pat. No. 8,586,713). Along with a universal light chain containing plasmid, the expression plasmids can be transfected into a host cell, such as a CHO cell. The host cells can then produce multispecific antigen binding molecules described herein.

In an embodiment, a method for making a multispecific antigen binding molecule includes a method of purifying the molecule, e.g., by column chromatography, precipitation and/or filtration. The product of such a method also forms part of the present disclosure along with a pharmaceutical composition thereof.

Host cells comprising a multispecific antigen binding molecule of the present disclosure and/or a polynucleotide encoding immunoglobulin chains of such a molecule (e.g., in a vector) are also part of the present invention. Host cells include, for example, mammalian cells such as Chinese hamster ovary (CHO) cells and fungal cells such as Pichia cells (e.g., P. pastoris).

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

Antigen binding molecules used as controls in Examples 6 through 10 include:
A CD20×CD3 bispecific antibody (REGN1979) (U.S. Pat. No. 10,550,193) used as signal 1 along with a matched isotype control (REGN7540). Both REGN1979 and REGN7540 comprise hIgG4^{P-PVA} isotype (U.S. Pat. No. 9,359,437).
A second CD20×CD3 bispecific antibody (REGN2281) used as signal 1. REGN2281 comprises variable regions identical to REGN1979 with a hIgG4 isotype having S108P substitution (hIgG4P)
BCMA×CD3 (REGN5458), also used as signal 1 (U.S. Pat. No. 11,384,153).
Comparator 1: 4-1BB bivalent agonist (REGN4249) comprising the variable regions of antibody “10C7” (U.S. Pat. No. 7,288,638).
Anti-PD-1 antibody cemiplimab (REGN2810; LIBTAYOO) (U.S. Pat. No. 9,987,500).
isotype control to 4-1BB bivalent agonist (hIgG4P) (REGN1945); REGN1945 was also used as the isotype control for cemiplimab.
Bispecific 4-1BB×non-TAA isotype control (REGN13168).

Two signals, “signal 1” & “signal 2”, are required for proper T cell activation. “Signal 1” is induced by binding of the T cell receptor (TCR) on T cells to peptide-bound major histocompatibility complex (MHC) molecules on antigen presenting cells (APCs). “Signal 2” is provided by engaging co-stimulatory receptors on T cells. One such costimulatory receptor is 4-1BB receptor, which is an inducible type I membrane protein and member of the tumor necrosis factor receptor (TNFR) superfamily. Expression of 4-1BB receptor is induced on the surface of T-cells after antigen- or mitogen-induced activation. The activation of 4-1BB occurs via engagement with 4-1BBL, present on APCs. Therefore, activation of 4-1BB signaling provides a targeted approach to enhance existing TCR signaling.

### Cell Line Descriptions

The characteristics of the cell lines used in several of the following examples are provided below:

### NALM6 (ACL14036)

NALM6 clone is an acute lymphoblastic leukemia (ALL) cell line isolated from a 19-year old male [NALM6 clone G5 (ATCC, #CRL-3273)]. NALM6 cells are maintained in RPMI 1640+10% FBS+P/S/G; 37° C. 5% CO₂.

Staining to confirm expression

### NALM6/hPD-L1 (ACL16389)

NALM6 cells that were genetically engineered to stably express human PD-L1 (amino acids M1-T290 of accession number NP_054862.1). Cells are maintained in RPMI 1640+10% FBS+P/S/G+1 ug/ml Puro; 37° C. 5% CO₂.

Virus Transfection and Transduction

Staining to confirm expression

### HEK293 Parental (ACL2397)

A human embryonic kidney cell line isolated from a fetus [HEK-293 (ATCC, #CRL-1573)]. Referred to as HEK293 parental cell line. HEK293 cells are maintained in DMEM+10% FBS+P/S/G.

### HEK293/hCD20 (ACL14268)

A cell line made by stably transducing HEK293 (HZ) cells with human CD20 (Uniprot accession #: P11836, amino acids M1 to P297). Engineered line is maintained in DME+10% FBS+penicillin/streptomycin/glutamine (P/S/G)+100 μg/mL hygromycin @ 5% CO₂.

Virus Transfection/Production

Cell Line Transduction

Staining to confirm expression

### HEK293/hCD20/hCD38 (ACL14270)

A cell line made by stably transducing HEK293 (HZ) cells with human CD20 (Uniprot accession #: P11836, amino acids M1 to P297) and human CD38 (Uniprot accession #: P28907, amino acids M1 to 1300). Engineered line is maintained in DME+10% FBS+penicillin/streptomycin/glutamine (P/S/G)+500 μg/mL G418+100 μg/mL hygromycin @ 5% CO₂.

Virus Transfection/Production

Cell Line Transduction

Staining to confirm expression

### HEK293/h4-1BB (ACL7888)

A cell line made by stably transducing HEK293 (HZ) cells with human TNFRSF9 (4-1BB) (Accession #: NM_001561, amino acids M1 to L255). Engineered line is maintained in DME+10% FBS+penicillin/streptomycin/glutamine (P/S/G)+500 μg/mL G418 @ 5% CO₂.

Virus Transfection/Production

Cell Line Transduction

Staining to confirm expression

### HEK293/NFkB-Luc (ACL5021)

A cell line made by transfecting a Firefly Luciferase-IRES-GFP gene driven by five copies of NF-κB response element located upstream of the minimal TATA promoter. Positive cell line selection was performed by flow cytometry and a single clone, D9, was isolated.

Plasmid Generation.

Staining to confirm expression.

### HEK293/NFkB-Luc/h4-1BB (ACL11090)

A cell line made by stably transducing HEK293/NFkB-Luc cells with human TNFRSF9 (4-1BB) (Accession #: NM_001561, amino acids M1 to L255). Engineered line is maintained in DME+10% FBS+penicillin/streptomycin/glutamine (P/S/G)+500 μg/mL G418 @ 5% CO₂.

Virus Transfection/Production

Cell Line Transduction

Staining to confirm expression

### MOLP8 (ACL14198)

A human multiple myeloma line established from the peripheral blood of a 52-year-old Japanese man with multiple myeloma in 2002. Acquired through DSMZ #: ACC 569.

Staining to confirm expression

### OPM2 (ACL14164)

Established from the peripheral blood of a 56-year-old woman with multiple myeloma in leukemic phase in 1982. Acquired through DSMZ #: ACC50.

Staining to confirm expression

### Example 1. Generation and Screening of Anti-CD38 Antibodies and Anti-4-1BB Antibodies

Anti-CD38 antibodies were obtained by immunizing a genetically engineered mouse comprising DNA encoding human immunoglobulin heavy and kappa light chain variable regions with cells expressing CD38 or with DNA encoding CD38. The antibody immune response was monitored by a CD38-specific immunoassay. When a desired immune response was achieved splenocytes were harvested and fused with mouse myeloma cells to preserve their viability and form hybridoma cell lines. The hybridoma cell lines were screened and selected to identify cell lines that produce CD38-specific antibodies. Using this technique several anti-CD38 chimeric antibodies (i.e., antibodies possessing human variable domains and mouse constant domains) were obtained. In addition, several fully human anti-CD38 antibodies were generated from directly isolating antigen-positive B cells without fusion to myeloma cells, as described in US 2007/0280945A1.

Likewise, anti-4-1BB antibodies were obtained by immunizing a genetically engineered mouse comprising DNA encoding human immunoglobulin heavy and kappa light chain variable regions with cells expressing 4-1BB or with DNA encoding 4-1BB. The antibody immune response was monitored by a 4-1BB-specific immunoassay. When a desired immune response was achieved splenocytes were harvested and fused with mouse myeloma cells to preserve their viability and form hybridoma cell lines. The hybridoma cell lines were screened and selected to identify cell lines that produce 4-1BB-specific antibodies. Using this technique several anti-4-1BB chimeric antibodies (i.e., antibodies possessing human variable domains and mouse constant domains) were obtained. In addition, several fully human anti-4-1BB antibodies were generated from directly isolating antigen-positive B cells without fusion to myeloma cells, as described in US 2007/0280945A1.

The antibodies were characterized and selected for desirable characteristics, including affinity, selectivity, etc. If necessary, mouse constant regions were replaced with a desired human constant region, for example wild-type or modified IgG1 or IgG4 constant region, to generate a fully human anti-CD38 antigen binding molecule or fully human anti-4-1BB antigen binding molecule. While the constant region selected may vary according to specific use, high affinity antigen binding and target specificity characteristics reside in the variable region.

### Example 2. Heavy and Light Chain Variable Region Amino Acid and Nucleic Acid Sequences of Anti-CD38 Binding Arm

Table 1 sets forth the amino acid sequence identifiers of the heavy and light chain variable regions and CDRs of selected anti-CD38 antigen binding arms of the invention. The corresponding nucleic acid sequence identifiers are set forth in Table 2.

TABLE 1
Anti-CD38 Amino Acid Sequence Identifiers

| | HCVR | | | | LCVR | | | |
|---|---|---|---|---|---|---|---|---|
| Ab Designation | (VH-1) | HCDR1 | HCDR2 | HCDR3 | (VL-1) | LCDR1 | LCDR2 | LCDR3 |
| mAb26871P2 | 2 | 4 | 6 | 8 | 18 | 20 | 22 | 24 |
| | | | | | (3-20GL) | | GAS | |
| mAb26812P2 | 40 | 42 | 44 | 46 | 48 | 50 | 52 | 54 |
| | | | | | (1-39GL) | | AAS | |

TABLE 2
Anti-CD38 Nucleic Acid Sequence Identifiers

| | HCVR | | | | LCVR | | | |
|---|---|---|---|---|---|---|---|---|
| Ab Designation | (VH-1) | HCDR1 | HCDR2 | HCDR3 | (VL-1) | LCDR1 | LCDR2 | LCDR3 |
| mAb26871P2 | 1 | 3 | 5 | 7 | 17 | 19 | 21 | 23 |
| | | | | | (3-20GL) | | ggggcaagt | |
| mAb26812P2 | 39 | 41 | 43 | 45 | 47 | 49 | 51 | 53 |
| | | | | | (1-39GL) | | gctgcatcc | |

The anti-AD38 binding arms may comprise variable domain and CR sequences as set forth in Table 1 and a human Fc domain of isotype IgG4, IgG, etc. For certain applications or experiments the Fc domain may be a mouse Fc domain. As will be appreciated by a person of ordinary skill in the art, an antigen binding arm having a particular Fc isotype can be converted to an antigen binding arm with a different Fc isotype (e.g., an antigen binding molecule with a mouse IgG4 Fc can be converted to an antigen binding molecule with a human IgG1, etc.), but in any event, the variable domains (including the CDRs)—which are indicated by the numerical identifiers shown in Table 1—will remain the same, and the binding properties are expected to be identical or substantially similar regardless of the nature of the Fc domain.

### Example 3: Heavy and Light Chain Variable Region Amino Acid and Nucleic Acid Sequences of Anti-4-1BB Binding Arm

Table 3 sets forth the amino acid sequence identifiers of the heavy and light chain variable regions and CDRs of selected anti-4-1BB binding arms of the bispecific antibodies. The corresponding nucleic acid sequence identifiers are set forth in Table 4.

TABLE 3
Anti-4-1bb Amino Acid Sequence Identifiers

| | HCVR | | | | LCVR | | | |
|---|---|---|---|---|---|---|---|---|
| Ab Designation | (VH-2) | HCDR1 | HCDR2 | HCDR3 | (VL-2) | LCDR1 | LCDR2 | LCDR3 |
| mAb25921P | 10 | 12 | 14 | 16 | 18 | 20 | 22 | 24 |
| mAb25889P2 | 32 | 34 | 36 | 38 | 48 | 50 | 52 | 54 |
| mAb35333P2 | 62 | 64 | 66 | 68 | 48 | 50 | 52 | 54 |
| mAb25898P2 | 72 | 74 | 76 | 78 | 48 | 50 | 52 | 54 |
| mAb35388P2 | 86 | 88 | 90 | 92 | 48 | 50 | 52 | 54 |
| mAb35321P2 | 94 | 96 | 98 | 100 | 48 | 50 | 52 | 54 |

TABLE 4
Anti-4-1bb Nucleic Acid Sequence Identifiers

| | HCVR | | | | LCVR | | | |
|---|---|---|---|---|---|---|---|---|
| Ab Designation | (VH-2) | HCDR1 | HCDR2 | HCDR3 | (VL-2) | LCDR1 | LCDR2 | LCDR3 |
| mAb25921P | 9 | 11 | 13 | 15 | 17 | 19 | 21 | 23 |
| mAb25889P2 | 31 | 33 | 35 | 37 | 47 | 49 | 51 | 53 |
| mAb35333P2 | 61 | 63 | 65 | 67 | 47 | 49 | 51 | 53 |
| mAb25898P2 | 71 | 73 | 75 | 77 | 47 | 49 | 51 | 53 |
| mAb35388P2 | 85 | 87 | 89 | 91 | 47 | 49 | 51 | 53 |
| mAb35321P2 | 93 | 95 | 97 | 99 | 47 | 49 | 51 | 53 |

The anti-4-1BB antigen binding arms may comprise variable domain and CDR sequences as set forth in Table 3 and a human Fc domain of isotype IgG4, IgG1, etc. For certain applications or experiments the Fc domain may be a mouse Fc domain. As will be appreciated by a person of ordinary skill in the art, an antigen binding molecule having a particular Fc isotype can be converted to an antigen binding molecule with a different Fc isotype (e.g., an antigen binding molecule with a mouse IgG4 Fc can be converted to an antigen binding molecule with a human IgG1, etc.), but in any event, the variable domains (including the CDRs)—which are indicated by the numerical identifiers shown in Table 3—will remain the same, and the binding properties are expected to be identical or substantially similar regardless of the nature of the Fc domain.

### Example 4: Generation of Multispecific Antigen Binding Molecules that Bind CD38 and 4-1BB

The multispecific antigen binding molecules that bind CD38 and 4-1BB are also referred to herein as “anti-CD38×anti-4-1BB 1+2” or “anti-CD38×anti-4-1BB multispecific molecules”, or “anti-CD38/anti-4-1BB 1+2”, or “CD38×4-1BB multispecific molecules”. The anti-CD38 portion of the anti-CD38×anti-4-1BB multispecific molecule is useful for targeting tumor cells that express CD38, and the anti-4-1BB portion of the multispecific molecule is useful for activating T-cells.

Various 4-1BB Fabs (Fab3; heavy chain variable regions (HCVR) with heavy chain CH1 domain and light chain) binding to 4-1BB epitope 1 (ep1) or epitope 2 (ep2) were fused to the N-terminus of a 4-1BB VH domain from an existing IgG-like bispecific molecule targeting both 4-1BB and CD38.

DNA fragments encoding (i) various 4-1BB heavy chain variable regions (HCVR) (ii) heavy chain CH1 domain followed by linkers of varied lengths for connecting the heavy chain CH1 domain to a second 4-1BB heavy chain variable regions (HCVR) (iii) CD38 heavy chain variable regions (HCVR) were synthesized by Integrated DNA Technologies, Inc. (San Diego, California).

Mammalian expression vectors for individual heavy chains were created by InFusion Cloning (Takara Bio USA Inc.) following protocols provided by Takara Bio USA Inc. CD38 heavy chain variable regions (HCVR) (VH-1) were cloned into a heavy chain expression plasmid (CH1-1_CH2_CH3). 4-1BB heavy chain variable regions (HCVR) (VH-3) fused to a CH1 domain (CH1-3) with linkers of various length (linker) followed by another 4-1BB heavy chain variable regions (HCVR) (VH-2) were cloned into a heavy chain expression plasmid (CH1-2_CH2_CH3(*)) containing the star mutation (H435R, Y436F, EU numbering).

Recombinant CD38×4-11BB×4-1BB 1+2 N-Fab MBMs were produced in CHO cells after transfection with 3 expression plasmids (i) CD38 heavy chain plasmid (ii) 4-1BB+4-1BB heavy chain star plasmid (iii) a universal light chain containing plasmid. Stably transfected CHO cell pools were isolated after selection with 400 μg/ml hygromycin for 12 days. The CHO cell pools were used to produce the CD38×4-1BB×4-1BB 1+2 N-Fab MBMs which were subsequently purified as described previously (Sci Rep. 2015 Dec. 11; 5:17943).

A summary of the component parts of the antigen binding domains of selected multispecific antigen binding molecules made in accordance with this Example is set forth in Table 5. The respective nucleic acid sequence identifiers of the component parts are provided in Table 6. Tables 7 and 8 provide the component parts, polypeptide sequences and nucleic acid sequences, respectively, for the control bispecific antigen binding molecule.

TABLE 5
Component Parts of Selected 1 + 2 Formatted Antibodies-Amino Acid Sequence Identifiers

| | CD38 arm (Fab1) | | | | | 4-1BB-“IN” arm (Fab2) | | | | | 4-1BB “OUT” arm (Fab3) | |
|---|---|---|---|---|---|---|---|---|---|---|---|---|
| REGN | Ab | | | | | Ab | | | | | Ab | |
| No. | Designation | VH-1 | CDR1 | CDR2 | CDR3 | Designation | VH-2 | CDR1 | CDR2 | CDR3 | Designation | VH-3 |
| 7633 | 26812P2 | 40 | 42 | 44 | 46 | 25889P2 | 32 | 34 | 36 | 38 | 35388P2 | 86 |
| 7647 | 26812P2 | 40 | 42 | 44 | 46 | 35333P2 | 62 | 64 | 66 | 68 | 35333P2 | 62 |
| 7650 | 26812P2 | 40 | 42 | 44 | 46 | 25898P2 | 72 | 74 | 76 | 78 | 35321P2 | 94 |
| 9682 | 26812P2 | 40 | 42 | 44 | 46 | 35333P2 | 62 | 64 | 66 | 68 | 35333P2 | 62 |
| 9686 | 26812P2 | 40 | 42 | 44 | 46 | 35333P2 | 62 | 64 | 66 | 68 | 35333P2 | 62 |

| | | | | | | | | | Full Length Heavy | | |
|---|---|---|---|---|---|---|---|---|---|---|---|
| | | | | LCVR | | | | | and Light Chain | | |
| REGN | 4-1BB “OUT” arm (Fab3) | | | Ab | | | | | HC-4- | HC- | LCULC1- |
| No. | CDR1 | CDR2 | CDR3 | Designation | Vk | CDR1 | CDR2 | CDR3 | 1BB | CD38 | 39GL |
| 7633 | 88 | 90 | 92 | 1-39(PP) GL | 48 | 50 | 52 | 54 | 56 | 58 | 60 |
| 7647 | 64 | 66 | 68 | 1-39(PP) GL | 48 | 50 | 52 | 54 | 70 | 58 | 60 |
| 7650 | 96 | 98 | 100 | 1-39(PP) GL | 48 | 50 | 52 | 54 | 80 | 58 | 60 |
| 9682 | 64 | 66 | 68 | 1-39(PP) GL | 48 | 50 | 52 | 54 | 82 | 58 | 60 |
| 9686 | 64 | 66 | 68 | 1-39(PP) GL | 48 | 50 | 52 | 54 | 84 | 58 | 60 |

TABLE 6
Component Parts of Selected 1 + 2 Formatted Antibodies-Nucleic Acid Sequence Identifiers

| | CD38 arm (Fab1) | | | | | 4-1BB-“IN” arm (Fab2) | | | | | 4-1BB “OUT” arm (Fab3) | |
|---|---|---|---|---|---|---|---|---|---|---|---|---|
| REGN | Ab | | | | | Ab | | | | | Ab | |
| No. | Designation | VH-1 | CDR1 | CDR2 | CDR3 | Designation | VH-2 | CDR1 | CDR2 | CDR3 | Designation | VH-3 |
| 7633 | 26812P2 | 39 | 41 | 43 | 45 | 25889P2 | 31 | 33 | 35 | 37 | 35388P2 | 85 |
| 7647 | 26812P2 | 39 | 41 | 43 | 45 | 35333P2 | 61 | 63 | 65 | 67 | 35333P2 | 61 |
| 7650 | 26812P2 | 39 | 41 | 43 | 45 | 25898P2 | 71 | 73 | 75 | 77 | 35321P2 | 93 |
| 9682 | 26812P2 | 39 | 41 | 43 | 45 | 35333P2 | 61 | 63 | 65 | 67 | 35333P2 | 61 |
| 9686 | 26812P2 | 39 | 41 | 43 | 45 | 35333P2 | 61 | 63 | 65 | 67 | 35333P2 | 61 |

| | | | | | | | | | Full Length Heavy | | |
|---|---|---|---|---|---|---|---|---|---|---|---|
| | | | | | | | | | and Light Chain | | |
| REGN | 4-1BB “OUT” arm (Fab3) | | | LCVR | | | | | HC-4- | HC- | LCULC1- |
| No. | CDR1 | CDR2 | CDR3 | Ab Designation | Vk | CDR1 | CDR2 | CDR3 | 1BB | CD38 | 39GL |
| 7633 | 87 | 89 | 91 | 1-39(PP) GL | 47 | 49 | 51 | 53 | 55 | 57 | 59 |
| 7647 | 63 | 65 | 67 | 1-39(PP) GL | 47 | 49 | 51 | 53 | 69 | 57 | 59 |
| 7650 | 95 | 97 | 99 | 1-39(PP) GL | 47 | 49 | 51 | 53 | 79 | 57 | 59 |
| 9682 | 63 | 65 | 67 | 1-39(PP) GL | 47 | 49 | 51 | 53 | 81 | 57 | 59 |
| 9686 | 63 | 65 | 67 | 1-39(PP) GL | 47 | 49 | 51 | 53 | 83 | 57 | 59 |

TABLE 7
Component Parts of 1 + 1 Formatted Control Antibody- Amino Acid Sequence Identifiers

| | CD38 arm | | | | | 4-1BB-“IN” (Fab2) | | | | | 4-1BB-“OUT” (Fab3) | |
|---|---|---|---|---|---|---|---|---|---|---|---|---|
| REGN | Ab | | | | | Ab | | | | | Ab | |
| No. | Designation | VH-1 | CDR1 | CDR2 | CDR3 | Designation | VH-2 | CDR1 | CDR2 | CDR3 | Designation | VH-3 |
| 7150 | 26871P2 | 2 | 4 | 6 | 8 | NA | NA | NA | NA | NA | 25921P | 10 |

| | | | | LCVR | | | | | | | |
|---|---|---|---|---|---|---|---|---|---|---|---|
| REGN | 4-1BB-“OUT” (Fab3) | | | Ab | | | | | Full Length HC and LC | | |
| No. | CDR1 | CDR2 | CDR3 | Designation | Vk | CDR1 | CDR2 | CDR3 | HC | HC | LC |
| 7150 | 12 | 14 | 16 | 3-20 GL | 18 | 20 | 22 | 24 | 26 | 28 | 30 |

TABLE 8
Component Parts of 1 + 1 Formatted Control Antibody- Nucleic Acid Sequence Identifiers

| | CD38 arm | | | | | 4-1BB-“IN” (Fab2) | | | | | 4-1BB-“OUT” (Fab3) | |
|---|---|---|---|---|---|---|---|---|---|---|---|---|
| REGN | Ab | | | | | Ab | | | | | Ab | |
| No. | Designation | VH-1 | CDR1 | CDR2 | CDR3 | Designation | VH-2 | CDR1 | CDR2 | CDR3 | Designation | VH-3 |
| 7150 | 26871P2 | 1 | 3 | 5 | 7 | NA | NA | NA | NA | NA | 25921P | 9 |

| | | | | LCVR | | | | | | | |
|---|---|---|---|---|---|---|---|---|---|---|---|
| REGN | 4-1BB-“OUT” (Fab3) | | | Ab | | | | | Full Length HC and LC | | |
| No. | CDR1 | CDR2 | CDR3 | Designation | Vk | CDR1 | CDR2 | CDR3 | HC | HC | LC |
| 7150 | 11 | 13 | 15 | 3-20 GL | 17 | 19 | 21 | 23 | 25 | 27 | 29 |

### Example 5: Screening of Multispecific Antigen Binding Molecules

Constructs activating the 4-1BB signaling pathway were next identified by screening.

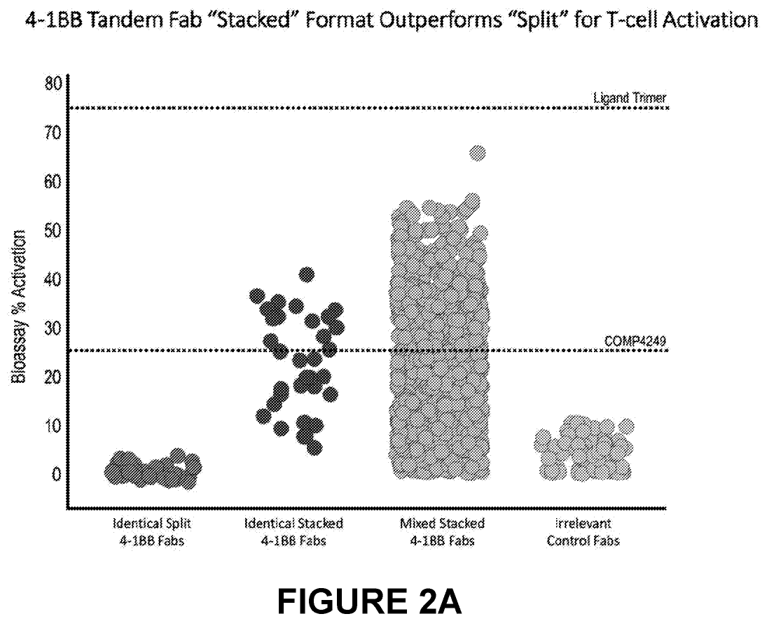

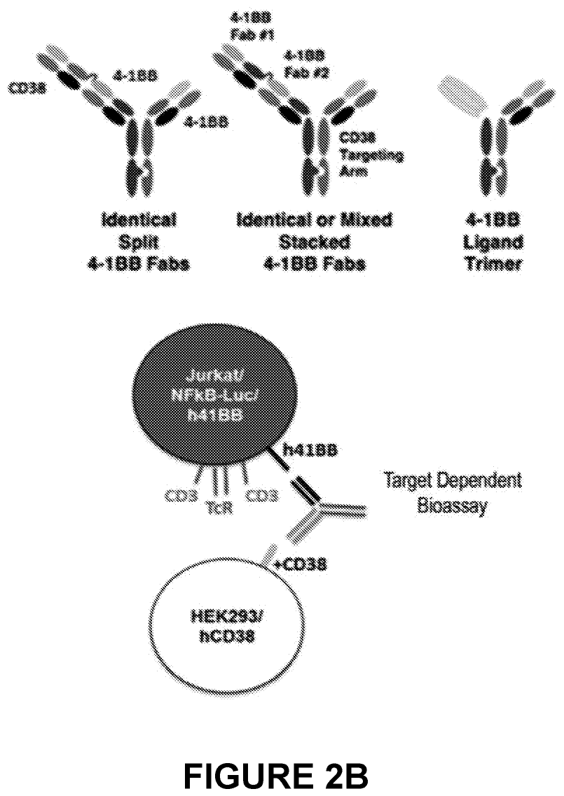

Thirty-four 4-1BB binding antibodies were selected for inclusion in the 1+2 screen based on cell binding to human 4-1BB, utilization of a common light chain and diversity of CDR3 sequences. For the screen, 4-1BB variable domains were arrayed in two different 1+2 formats, split 4-1BB or stacked 4-1BB. See FIG. 2B.

For the split format, a standard bivalent architecture for 4-1BB was maintained and tumor targeting was achieved by adding the variable domain and CH1 from a CD38 tumor targeting arm (26812) at the N-terminus of one side of the 4-1BB molecule separated by a G4S×3 spacer domain. These constructs were expressed as Knob in Hole (KiH) bispecifics so only one arm of the expressed molecule contained the CD38 targeting motif.

For the stacked FAB format, tandem 4-1BB sequences were linked in the same manner as CD38×4-1BB described above, and tumor targeting was achieved by expression as a bispecific molecule with anti-CD38 present on the opposing arm. The thirty-four 4-1BB variable domains along with one irrelevant control sequence (anti-BetV1) were assembled in all possible combinations of tandem FABs generating 1225 combinations. This included thirty-four molecules where the two stacked 4-1BB variable sequences were identical.

For screening, the 1+2 constructs were transiently expressed in CHO cells and bispecific containing supernatants harvested 4 days later. Supernatants were added to a co-culture of cells containing HEK cells over-expressing hCD38 and Jurkat cells harboring an NFkB luciferase reporter and over-expressing human 4-1BB. Engagement and activation of 4-1BB by the test samples was compared to a co-culture of the Jurkat reporter cells with HEK cells expressing the 4-1BB ligand (100%). Screening results from split FABs showed little to no activity in the Jurkat reporter assay with the highest activity observed to be 3.7%. In contrast, robust activation was observed with the stacked FAB format with over twenty-five percent of the samples tested yielding activities greater than 30%. Activity was observed when the 4-1BB variable domains within the stacked FAB were identical or unique sequences and activities as high as 66% were observed. Introduction of the irrelevant control FAB at any location within the stacked FAB resulted in a loss of activity. See FIG. 2A.

### Example 6: Characterization of CD38×4-1BB Multispecific Antigen Binding Molecules Binding Using Flow Cytometry

Binding of the CD38 arm was tested using MOLP8 cells which endogenously express CD38. Binding of the 4-1BB arm was assessed using HEK293 cells engineered to express h4-1BB (HEK293/h4-1BB). HEK293 cells were used to determine non-targeted cell binding, as they do not express CD38 nor 4-1BB.

The ability of the multispecific antigen binding molecules to bind cells was assessed using flow cytometry. Cell lines were chosen to determine the ability of both the anti-CD38 and anti-4-1BB arms to bind their targets. In the first experiment test antibodies were incubated with MOLP8 (endogenously express hCD38) and HEK293 (do not express hCD38) cells. In the second experiment test antibodies were incubated with HEK293/h4-1BB (engineered to express h4-1BB) and HEK293 (do not express h4-1BB) cells. Binding was detected by using a labeled secondary antibody and measuring fluorescence on a flow cytometer.

### Multispecific Antigen Binding Molecules:

Multispecific antigen binding molecules and the controls tested in these two experiments are as shown in Table 9.

TABLE 9
CD38x4-1BB Binding Molecules and Controls

| MABM Identifier | Description |
|---|---|
| REGN7633 | CD38x4-1BB (1 + 2), (hIgG4^{P-PVA}) |
| REGN7647 | CD38x4-1BB (1 + 2), (hIgG4^{P-PVA}) |
| REGN7650 | CD38x4-1BB (1 + 2), (hIgG4^{P-PVA}) |
| REGN7150 | CD38x4-1BB (1 + 1), (hIgG4^{P-PVA}) |
| REGN4249 | Comparator 1 |
| REGN7540 | Isotype control (hIgG4^{P-PVA}) |
| REGN1945 | Isotype control (hIgG4^{P}) |

### Experiment 1—CD38 Binding:

HEK293 cells were lifted with trypsin, washed and resuspend in stain buffer (2% FBS in PBS). MOLP8 cells were washed and resuspended in stain buffer. Cells were added to wells of a 96 well V-bottom plate (3×10⁵ cells/well). A 1:4, 9-point, dose titration of test antibodies was diluted in stain buffer and added to cells to a final concentration ranging from 610 fM to 10 nM (a “no antibody” control was included as the ninth point (153fM), labeled as “secondary only”). Cells and antibodies were incubated for 30 min on ice and then washed in stain buffer. Cells were resuspended in 2 ug/ml allophycocyanin (APC) conjugated goat-anti human secondary antibody and incubated for 30 min on ice. Cells were then washed and resuspended in viability dye (according to the manufacturer's protocol) and incubated for 30 min on ice. Cells were then washed with stain buffer and resuspended in 2% PFA for 30 min on ice. After washing, cells were filtered and analyzed by flow cytometry to determine the geometric mean fluorescence intensity (gMFI), which was subsequently plotted using GraphPad Prism software. EC₅₀ values of the antibodies were determined from a 4-parameter logistic equation over a 9-point dose response curve (including secondary only control, representing the ninth point). Results are shown in Table 10.

Dose dependent binding of the CD38×4-1BB 1+2 (REGN7633, REGN7647 and REGN7650) and 1+1 (REGN7150) multispecific antigen binding molecules was observed in the presence of MOLP8 cells, which endogenously express CD38. However, no binding was observed for the bivalent anti-4-1BB antibody REGN4249. Nor was binding observed for the isotype control antibodies (REGN7540 and REGN1945).

No binding of the CD38×4-1BB 1+2 antibody REGN7633 or the isotype control antibodies (REGN7540 and REGN1945) was observed in the presence of HEK293 cells, which do not express CD38. Weak binding (>100-fold lower gMFI than on the MOLP8 cells) was observed with the CD38×4-1BB 1+2 (REGN7647 and REGN7650) and 1+1 bispecific (REGN7150) antibodies as well as the bivalent anti-4-1BB antibody REGN4249.

TABLE 10
Maximum Binding and EC50 Values of Binding for Antibodies

| | |
|---|---|
| MOLP8 | HEK293 |

| | MAX | EC50 | MAX | EC50 |
|---|---|---|---|---|
| | [gMFI] | [M] | [gMFI] | [M] |
| REGN7633 | 8.8E+04 | NC | 7.1E+02 | ND |
| REGN7647 | 1.2E+05 | NC | 1.2E+03 | 2.43E−10 |
| REGN7650 | 1.4E+05 | NC | 9.5E+02 | ND |
| REGN7150 | 2.1E+05 | NC | 1.1E+03 | NC* |
| REGN4249 | 5.3E+02 | ND | 1.4E+03 | NC* |
| REGN7540 | 4.6E+02 | ND | 7.4E+02 | ND |
| REGN1945 | 4.5E+02 | ND | 7.5E+02 | ND |

Abbreviations: ND: Not Determined; NC: Not calculated because the data did not fit a 4-parameter logistic equation.
*Most likely due to nonspecific binding

### Experiment 2-4-1BB Binding:

HEK293 and HEK293/4-1BB cells were lifted with trypsin, washed and resuspended in stain buffer (2% FBS in PBS) and added to wells of a 96 well V-bottom plate (3×10⁵ cells/well). A 1:5, 9-point, dose titration of test antibodies was diluted in stain buffer and added to cells to a final concentration ranging from 1.3 pM to 100 nM (a “no antibody” control was included as the ninth point (0.26 nM) labeled as “secondary only”). Cells and antibodies were incubated for 30 min on ice and then washed in stain buffer. Cells were resuspended in 2 ug/ml allophycocyanin (APC) conjugated goat-anti human secondary antibody and incubated for 30 min on ice. Cells were then washed and resuspended in viability dye (according to the manufacturer's protocol) and incubated for 30 min on ice. Cells were then washed with stain buffer and resuspended in 2% PFA for 30 min on ice. After washing, cells were filtered and analyzed by flow cytometry to determine the geometric mean fluorescence intensity (gMFI), which was subsequently plotted using GraphPad Prism software. EC₅₀ values of the antibodies were determined from a 4-parameter logistic equation over a 9-point dose response curve (including secondary only control). Results are shown in Table 11.

Dose dependent binding of the CD38×4-1BB 1+2 (REGN7633, REGN7647 and REGN7650) and 1+1 (REGN7150) multispecific antigen binding molecules was observed in the presence of HEK293/h4-1BB cells, which were engineered to express h4-1BB. Binding of the bivalent anti-4-1BB antibody REGN4249 was also observed. Binding was not observed for the isotype control antibodies (REGN7540 and REGN1945).

No binding of the CD38×4-1BB 1+2 antibody REGN7633 or the isotype control antibodies (REGN7540 and REGN1945) was observed in the presence of HEK293 cells, which do not express h4-1BB. Slight binding (more than 1,000-fold lower gMFI than on the HEK293/h4-1BB cells) was observed with the CD38×4-1BB 1+2 multispecific antigen binding molecules (REGN7647 and REGN7650), the CD38×4-1BB bispecific (REGN7150), and the bivalent anti-4-1BB antibody REGN4249.

TABLE 11
Maximum Binding and EC50 Values of Binding for Antibodies

| | HEK293/4-1BB | | HEK293 | |
|---|---|---|---|---|
| | MAX | EC50 | MAX | EC50 |
| | [gMFI] | [M] | [gMFI] | [M] |
| REGN7633 | 8.9E+05 | 1.47E−08 | 1.6E+04 | ND |
| REGN7647 | 1.4E+06 | 6.08E−10 | 1.8E+04 | ND |
| REGN7650 | 1.0E+06 | 9.97E−10 | 2.4E+04 | NC* |
| REGN7150 | 2.4E+06 | 2.86E−09 | 2.5E+04 | 2.72E−09* |
| REGN4249 | 7.4E+05 | 8.86E−10 | 1.8E+04 | ND |
| REGN7540 | 2.5E+04 | ND | 1.9E+04 | ND |
| REGN1945 | 1.6E+04 | ND | 1.4E+04 | ND |

Abbreviations: ND: Not Determined; NC: Not calculated because the data did not fit a 4-parameter logistic equation.
*Most likely due to nonspecific binding

### Example 7: Characterization of CD38×4-1BB Bispecific Antibodies in T-Cell Activation Assays Using HEK293/hCD20/hCD38, HEK293/hCD20, MOLP8 and Human Primary T-Cells

Two signals, “signal 1” & “signal 2”, are required for proper T cell activation. “Signal 1” is induced by binding of the T cell receptor (TCR) on T cells to peptide-bound major histocompatibility complex (MHC) molecules on antigen presenting cells (APCs). Whereas, “signal 2” is provided by engaging co-stimulatory receptors on T cells. One such costimulatory receptor is 4-1BB receptor, which is an inducible type I membrane protein and member of the tumor necrosis factor receptor (TNFR) superfamily. Expression of 4-1BB receptor is induced on the surface of T-cells after antigen- or mitogen-induced activation. The activation of 4-1BB occurs via engagement with 4-1BBL, present on APCs. Therefore, activation of 4-1BB signaling provides a targeted approach to enhance existing TCR signaling.

CD38×4-1BB (1+1 and 1+2) bispecific antibodies are designed to mimic the natural ligand of 4-1BB, by bridging CD38⁺ target cells with 4-1BB receptor positive T cells, to provide “signal 2” in order to enhance the activation of T cells in the presence of a “signal 1” provided by a Tumor-associated antigen (TAA)×CD3 bispecific antibody or an allogeneic response provided by the APC.

### Multispecific Antigen Binding Molecules:

Multispecific antigen binding molecules and the controls tested in this experiment are as shown in Table 12.

TABLE 12
CD38x4-1BB Binding Molecules and Controls

| MABM Identifier | Description |
|---|---|
| REGN7633 | CD38x4-1BB (1 + 2), (hIgg4^{^{P-PVA}}) |
| REGN7647 | CD38x4-1BB (1 + 2), (hIgg4^{P-PVA}) |
| REGN7650 | CD38x4-1BB (1 + 2), (hIgg4^{P-PVA}) |
| REGN7150 | CD38x4-1BB (1 + 1), (hIgg4^{P-PVA}) |
| REGN4249 | Comparator 1 |
| REGN7540 | Isotype control, (hIgg4^{P-PVA}) |
| REGN1945 | Isotype control, (hIgg4^{P}) |
| REGN5458 | BCMAxCD3, (hIgg4^{P-PVA}) |
| REGN1979 | CD20xCD3, (hIgg4^{P-PVA}) |

The ability of CD38×4-1BB multispecific antigen binding molecules to activate human primary T-cells by engaging CD38 and 4-1BB receptor to deliver “signal 2”, as determined by IL-2 release, was evaluated in the presence of a human embryonic kidney cancer cell line engineered to express hCD20 and hCD38 (HEK293/hCD20/hCD38) using REGN1979 (CD20×CD3) to serve as “signal 1.” HEK293 cells expressing only hCD20 were included as a control to measure activity that may occur in the absence of CD38 on APC's. Additionally, a multiple myeloma cell line that endogenously expresses hCD38, MOLP8, was included in testing CD38×4-1BB bispecific antibodies. As MOLP8 cells endogenously express BCMA, REGN5458 (BCMA×CD3) was included to serve as “signal 1.” Of note, unlike HEK293 cells, MOLP8 cells are able to provide detectable allogeneic stimulation of T-cells, serving as “signal 1’, in the absence CD3 stimulation provided by REGN5458.

### Isolation of Human Primary CD3⁺ T Cells:

Human peripheral blood mononuclear cells (PBMCs) were isolated from a healthy donor leukocyte pack from Precision for Medicine (Donor 555105) using density gradient centrifugation. Briefly, 15 ml of Ficoll-Paque PLUS is added to 50 ml conical tubes, and subsequently 30 ml of blood diluted 1:1 with PBS containing 2% FBS is layered on top. After a 30-minute centrifugation at 400×g, with the brake off, the buffy coat (containing mononuclear cells) is transferred to a fresh tube, diluted 5× with PBS containing 2% FBS and centrifuged for 8 minutes at 300×g. Subsequently, CD3⁺ T-cells were isolated from PBMC's using an EasySep™ Human CD3⁺ T Cell Isolation Kit from StemCell Technologies and following the manufacturer's recommended instructions.

### IL-2 Release Assay:

Enriched CD3⁺ T-cells, resuspended in stimulation media, were added into 96-well round bottom plates at a concentration of 1×10⁵ cells/well. Growth-arrested HEK293/hCD20/hCD38 or HEK293/hCD20 were added to CD3⁺ T-cells at a final concentration of 1×10⁴ cells/well. Growth-arrested MOLP8 cells were added to CD3⁺ T-cells at a final concentration of 5×10⁴ cells/well. Following addition of cells, a constant of 0.1 nM REGN1979 or its matched isotype control (REGN7540) was added to wells containing HEK293/hCD20/hCD38 or HEK293/hCD20. A constant of 0.5 nM REGN5458 or an isotype control was added to wells containing MOLP8 cells. Subsequently, CD38×4-1BB (1+1 or 1+2), bivalent 4-1BB (REGN4249), or isotype controls (REGN7540 or REGN1945) were titrated from 3 pM to 200 nM in a 1:4 dilution and added to wells. The final point of the 10-point dilution contained no titrated antibody. Plates were incubated for 72 hours at 37° C., 5% CO₂ and 5 μL total supernatant was used for measuring IL-2. The amount of cytokine in assay supernatant was determined using AlphaLisa kits from PerkinElmer following the manufacturer's protocol. The cytokine measurements were acquired on Perkin Elmer's multilabel plate reader Envision and values were reported as μg/mL. All serial dilutions were tested in duplicate.

The EC₅₀ values of the antibodies were determined from a four-parameter logistic equation over a 10-point dose-response curve using GraphPad Prism™ software. Maximal IL-2 is given as the mean max response detected within the tested dose range. Results are provided in Table 13.

Results with HEK293/hCD20 & HEK293/hCD20/hCD38 Cell Lines

In the presence of target and “signal 1”, provided by REGN1979, 4-1BB antibody treatment led to higher IL-2 response compared to matched isotype controls (REGN7540 and REGN1945). Of note, 1+2 formats of CD38×4-1BB led to higher maximum IL-2 and greater potency, compared to 1+1 CD38×4-1BB (REGN7150) and bivalent 4-1BB (REGN4249) antibody. In the absence of CD38 target only bivalent 4-1BB (REGN4249) exhibited a dose dependent increase in IL-2 release. In the absence of ‘signal 1’ none of the antibodies lead to dose dependent enhancement of IL-2 release.

Results with MOLP8 Cell Line

In the presence of allogeneic MOLP8 cells, and absence of REGN5458, there was a dose-dependent increase in IL-2 observed for 1+2 CD38×4-1BB antibodies REGN7647 and REGN7650 and to a lesser extent for 1+2 CD38×4-1BB antibody REGN7633 and bivalent 4-1BB antibody REGN4249. The 1+1 CD38×4-1BB and isotype control antibodies did not exhibit dose dependent enhancement of IL-2 release. While “signal 1” can be provided by allogeneic MOLP8 cells, the addition of REGN5458, was also evaluated. Under these conditions all 4-1BB antibodies led to dose dependent increases in IL-2 release compared to matched isotype control, with the 1+2 CD38×4-1BB antibodies exhibiting the greatest potency and maximum increase in IL-2.

TABLE 13
Maximum IL-2 release and Potency Values of Antibodies

| | HEK293/CD20/CD38 + | | HEK293/CD20 + | | HEK293/CD20/CD38 + | | MOLP-8 + | | MOLP-8 + | |
|---|---|---|---|---|---|---|---|---|---|---|
| | REGN1979 | | REGN1979 | | IsoC | | IsoC | | REGN5458 | |
| | MAX | EC50 | MAX | EC50 | MAX | EC50 | MAX | EC50 | MAX | EC50 |
| | [pg/ml] | [M] | [pg/ml] | [M] | [pg/ml] | [M] | [pg/ml] | [M] | [pg/ml] | [M] |
| REGN7633 | 1934 | 8.3E−11 | 218 | 2.16E−09 | 16 | ND | 177 | 1.31E−11 | 534 | 4.482E−10 |
| REGN7647 | 2847 | 5.8E−12 | 199 | NC | 18 | ND | 331 | 2.21E−12 | 692 | 2.31E−11 |
| REGN7650 | 2721 | 1.4E−11 | 184 | NC | 10 | ND | 288 | NC | 553 | 3.166E−11 |
| REGN7150 | 1303 | 1E−10 | 182 | ND | 12 | ND | 163 | ND | 308 | 6.338E−08 |
| REGN4249 | 931 | 1.4E−09 | 729 | 1.42E−09 | 15 | ND | 174 | NC | 183 | 5.883E−10 |
| REGN7540 | 176 | ND | 195 | ND | 9 | ND | 170 | ND | 156 | ND |
| REGN1945 | 173 | ND | 208 | ND | 10 | ND | 155 | ND | 164 | ND |

Abbreviations: ND: Not Determined; NC: Not calculated because the data did not fit a 4-parameter logistic equation.

### Example 8: Characterization of CD38×4-1BB (1+2) Multispecific Antigen Binding Molecules in T-Cell Activation Assays Using HEK293/hCD20/hCD38, HEK293/hCD20, MOLP8, NALM6, and Human Primary T-Cells

Two signals, “signal 1” & “signal 2”, are required for proper T cell activation. “Signal 1” is induced by binding of the T cell receptor (TCR) on T cells to peptide-bound major histocompatibility complex (MHC) molecules on antigen presenting cells (APCs). “Signal 2” is provided by engaging co-stimulatory receptors on T cells. One such costimulatory receptor is 4-11BB receptor, which is an inducible type I membrane protein and member of the tumor necrosis factor receptor (TNFR) superfamily. Expression of 4-1BB receptor is induced on the surface of T-cells after antigen- or mitogen-induced activation. The activation of 4-1BB occurs via engagement with 4-1BBL, present on APCs. Therefore, activation of 4-1BB signaling provides a targeted approach to enhance existing TCR signaling.

CD38×4-1BB (1+1 and 1+2) bispecific antibodies are designed to mimic the natural ligand of 4-1BB, by bridging CD38⁺ target cells with 4-1BB receptor positive T cells, to provide “signal 2” in order to enhance the activation of T cells in the presence of a “signal 1” provided by a Tumor-associated antigen (TAA)×CD3 bispecific antibody or an allogeneic response provided by the APC.

### Multispecific Antigen Binding Molecules:

Multispecific antigen binding molecules and the controls tested in this experiment are as shown in Table 14.

TABLE 14
CD38x4-1BB Binding Molecules and Controls

| MABM Identifier | Description, Linker, Isotype |
|---|---|
| REGN7647 | CD38x4-1BB (1 + 2), (G4S)3v15 (hIgg4^{P-PVA}) |
| REGN9682 | CD38x4-1BB (1 + 2), (G4S)3 (hIgg4^{P-PVA}) |
| REGN9686 | CD38x4-1BB (1 + 2), (G4S)2 (hIgg4^{P-PVA}) |
| REGN7150 | CD38x4-1BB (1 + 1), (hIgg4^{P-PVA}) |
| REGN13168 | Non-TAAx4-1BB (1 + 2) Control, (hIgg4^{P-PVA}) |
| REGN4249 | Comparator 1 |
| REGN5458 | BCMAxCD3, (hIgg4^{P-PVA}) |
| REGN2281 | CD20xCD3, (hIgg4^{P}) |
| REGN7540 | Isotype control, (hIgg4^{P-PVA}) |
| REGN1945 | Isotype Control, (hIgg4^{P}) |

The ability of CD38×4-1BB 1+2 multispecific antigen binding molecules, harboring different linkages between 2 tandem 4-1BB binding Fab domains, to activate human primary T-cells by engaging CD38 and 4-1BB receptor to deliver “signal 2”, as determined by IL-2 or IFNγ release, was evaluated in the presence of a human embryonic kidney cancer cell line engineered to express hCD20 and hCD38 (HEK293/hCD20/hCD38) using REGN2281 (CD20×CD3) to serve as “signal 1.” HEK293 cells expressing only hCD20 were included as a control to measure activity that may occur in the absence of CD38 on APC's. Additionally, a multiple myeloma cell line that endogenously expresses hCD38, MOLP8, was included in testing CD38×4-1BB (1+2) bispecific antibodies. As MOLP8 cells endogenously express BCMA, REGN5458 (BCMA×CD3) was included to serve as “signal 1.” Lastly, conditions that included either MOLP8 or NALM-6 (an acute lymphoblastic leukemia cell line that endogenously expresses CD38) as target cells, in the absence of a CD3 bispecific were tested. Of note, unlike HEK293 cells, MOLP8 and NALM-6 cells are able to provide detectable allogeneic stimulation of T-cells, serving as “signal 1’, in the absence CD3 antibody stimulation.

### Isolation of Human Primary CD3⁺ T Cells:

Human peripheral blood mononuclear cells (PBMCs) were isolated from a healthy donor leukocyte pack from Precision for Medicine (Donor 555114) using an EasySep Human T-Cell Isolation kit from StemCell Technologies, following the manufacturer's recommendations. Subsequently, CD3⁺ T-cells were isolated from PBMC's using an EasySep™ Human CD3⁺ T Cell Isolation Kit from StemCell Technologies and following the manufacturer's recommended instructions.

### Primary T-Cell Activation Assay:

Enriched CD3⁺ T-cells, resuspended in stimulation media, were added into 96-well round bottom plates at a concentration of 1×10⁵ cells/well. Target cells were added to CD3⁺ T-cells at a final concentration of 1×10⁴ cells/well for HEK293/hCD20/hCD38 or HEK293/hCD20 cells or 5×10⁴ cells/well for MOLP8 and NALM-6 cells. Following addition of cells, a constant of 0.25 nM REGN2281 was added to wells containing HEK293/hCD20/hCD38 or HEK293/hCD20 cells. A constant of 0.5 nM REGN5458 or an isotype control was added to wells containing MOLP8 cells. No CD3 bispecific was added to wells containing NALM-6 target cells. Subsequently, CD38×4-1BB (1+1 or 1+2), bivalent 4-1BB (REGN4249), or isotype controls (REGN7540 or REGN1945) were titrated from 128 fM to 50 nM in a 1:5 dilution and added to wells. The final point of the 10-point dilution contained no titrated antibody. Plates were incubated for 72 hours at 37° C., 5% CO₂ and 5 μL total supernatant was used for measuring IL-2 or IFNγ. The amount of cytokine in assay supernatant was determined using AlphaLisa kits from PerkinElmer following the manufacturer's protocol. The cytokine measurements were acquired on Perkin Elmer's multilabel plate reader Envision and values were reported as μg/mL. All serial dilutions were tested in duplicate.

The EC₅₀ values of the antibodies were determined from a four-parameter logistic equation over a 10-point dose-response curve using GraphPad Prism™ software. Maximal cytokine is given as the mean max response detected within the tested dose range. Results are provided in Tables 15 and 16.

### HEK293/hCD20 & HEK293/hCD20/hCD38

In the presence of target and “signal 1”, provided by REGN2281, 4-1BB antibody treatment led to higher IL-2 and IFNγ response compared to matched isotype IgG4^{P-PVA}, IgG4^{P}, or bispecific antibody controls (REGN7540, REGN1945 and REGN13168, respectively). Of note, 1+2 formats of CD38×4-1BB led to higher maximum cytokine release and greater potency, compared to 1+1 CD38×4-1BB (REGN7150) and bivalent 4-1BB (REGN4249) antibody, with similar level and potency of cytokine release observed regardless of the linkage between the 2 tandem 4-1BB binding Fab domains. In the absence of CD38 target, only bivalent 4-1BB (REGN4249) exhibited a dose dependent increase in cytokine release.

### MOLP8

In the presence of allogeneic MOLP8 cells, and absence of CD3 bispecific antibody stimulation, 1+2 CD38×4-1BB antibodies REGN7647, REGN9682, and REGN9686, mediated a dose-dependent increase in IL-2 and IFNγ. In comparison, the 1+1 CD38×4-1BB antibody, REGN7150, and bivalent 4-1BB antibody, REGN4249, led to minor dose dependent increases in IL-2 and not IFNγ. No dose dependent increase was observed for isotype and non-targeted 1+2 4-1BB control antibodies for either IL-2 or IFNγ. Addition of a fixed amount of REGN5458 (BCMA×CD3) in conditions with MOLP8 cells and primary human T-cells resulted in a dose dependent increase in IL-2 and IFNγ for all CD38-targeted 4-1BB antibodies as well as for REGN4249. As noted previously, the 1+2 CD38×4-1BB antibodies exhibited greater potency and maximum cytokine increase in comparison to REGN7150 and REGN4249, with the format of linkage between the 4-1BB-binding Fabs having little impact on potency or maximum cytokine release. Isotype control and non-TAA×4-1BB 1+2 control antibodies did not lead to dose dependent cytokine release.

### NALM-6

In the presence of allogeneic NALM-6 cells, and absence of CD3 bispecific antibody stimulation, all CD38-targeted 4-1BB antibodies (REGN7647, REGN9682, REGN9686, and REGN7150) as well as the bivalent 4-1BB antibody (REGN4249), led to dose-dependent increases in IFNγ and IL-2. The 1+2 CD38×4-1BB antibodies REGN7647, REGN9682, and REGN9686 exhibited greater potency and maximum cytokine release compared to the 1+1 CD38×4-1BB antibody, REGN7150, and bivalent 4-1BB antibody, REGN4249. As noted previously all 1+2 CD38×4-1BB multispecific antigen binding molecules performed similarly, regardless of the linker between the 4-1BB-binding Fabs. Isotype control and non-TAA×4-1BB 1+2 control antibodies did not lead to dose dependent cytokine release.

TABLE 15
Maximum IL2 Release and Potency Values

| | HEK293/CD20/CD38 + | | HEK293/CD20 + | | MOLP-8 + | | MOLP-8 + | | | |
|---|---|---|---|---|---|---|---|---|---|---|
| | REGN2281 | | REGN2281 | | REGN5458 | | IsoC | | NALM-6 | |
| | MAX | EC50 | MAX | EC50 | MAX | EC50 | MAX | EC50 | MAX | EC50 |
| | [pg/ml] | [M] | [pg/ml] | [M] | [pg/ml] | [M] | [pg/ml] | [M] | [pg/ml] | [M] |
| REGN7647 | 781 | 5.35E−11 | 61 | ND | 66 | 2.16E−10 | 73 | NC | 192 | 4.85E−11 |
| REGN9682 | 912 | 6.07E−11 | 52 | ND | 79 | 1.33E−10 | 96 | NC | 205 | 7.17E−11 |
| REGN9686 | 709 | 4.53E−11 | 57 | ND | 62 | 8.33E−11 | 90 | 3.61E−11 | 169 | 7.23E−11 |
| REGN7150 | 593 | NC | 63 | ND | 32 | NC | 48 | NC | 122 | 1.08E−10 |
| REGN13168 | 41 | ND | 57 | ND | 16 | ND | 28 | ND | 48 | ND |
| REGN4249 | 110 | NC | 126 | 8.19E−10 | 19 | NC | 54 | NC | 108 | NC |
| REGN7540 | 37 | ND | 54 | ND | 17 | ND | 21 | ND | 35 | ND |
| REGN1945 | 48 | ND | 63 | ND | 18 | ND | 33 | ND | 41 | ND |

Abbreviations: NC, not calculated because the data did not fit a 4-parameter logistic equation; ND, not determined because a concentration-dependent increase was not observed.

TABLE 16
Maximum IFNγ Release and Potency Values

| | HEK293/CD20/CD38 + | | HEK293/CD20 + | | MOLP-8 + | | MOLP-8 + | | | |
|---|---|---|---|---|---|---|---|---|---|---|
| | REGN2281 | | REGN2281 | | REGN5458 | | IsoC | | NALM-6 | |
| | MAX | EC50 | MAX | EC50 | MAX | EC50 | MAX | EC50 | MAX | EC50 |
| | [pg/ml] | [M] | [pg/ml] | [M] | [pg/ml] | [M] | [pg/ml] | [M] | [pg/ml] | [M] |
| REGN7647 | 6046 | 6.17E−11 | 1908 | ND | 3284 | 7.07E−11 | 103 | NC | 655 | 7.03E−11 |
| REGN9682 | 5271 | 5.26E−11 | 1573 | ND | 3406 | 7.25E−11 | 161 | NC | 658 | 4.19E−11 |
| REGN9686 | 6823 | 8.74E−11 | 1449 | ND | 3891 | 1.02E−10 | 85 | NC | 651 | NC |
| REGN7150 | 3951 | 7.80E−10 | 1843 | ND | 1297 | 4.38E−08 | 56 | NC | 620 | NC |
| REGN13168 | 1805 | ND | 1638 | ND | 656 | ND | 1 | ND | 102 | ND |
| REGN4249 | 2474 | 6.40E−10 | 2279 | 4.29E−09 | 1003 | NC | 37 | NC | 400 | NC |
| REGN7540 | 2080 | ND | 1455 | ND | 602 | ND | 1 | ND | 130 | ND |
| REGN1945 | 2086 | ND | 1664 | ND | 542 | ND | 83 | NC | 117 | ND |

Abbreviations: NC, not calculated because the data did not fit a 4-parameter logistic equation; ND, not determined because a concentration-dependent increase was not observed.

### Example 9: Characterization of CD38×4-1BB (1+2) Multispecific Antigen Binding Molecules in an Engineered Reporter Assay Using HEK293/hCD20/hCD38, HEK293/hCD20, MOLP8, OPM2 and HEK293/NFkB-Luc/h4-1BB Cells

The ability of CD38×4-1BB multispecific antigen binding molecules to specifically activate 4-1BB receptor in the presence of target cells expressing CD38 was measured in an engineered reporter assay. In this assay, engineered HEK293 cells express the reporter gene luciferase under the control of the transcription factor NF-KB (NFKB-Luc) along with the costimulatory receptor 4-1BB (HEK293/NFkB-Luc/h4-1BB). The target cells used in this assay were HEK293 cells engineered to express CD20 alone or in combination with CD38 or cell lines that endogenously express CD38, namely OPM2 and MOLP8. The ability of 4-1BB arms to stimulate 4-1BB activity is assessed by combining reporter cells with target cells and a titration of CD38×4-1BB 1+2 antibody. Activation of 4-1BB results in NFKB-driven luciferase production, which is then measured via a luminescence readout. In these assays the impact of different types of linkages between the 2 tandem 4-1BB targeting domains was also evaluated.

### Multispecific Antigen Binding Molecules:

Multispecific antigen binding molecules and the controls tested in this experiment are as shown in Table 17.

TABLE 17
CD38x4-1BB Binding Molecules and Controls

| MABM Identifier | Description |
|---|---|
| REGN7647 | CD38x4-1BB (1 + 2), (hIgg4^{P-PVA}) |
| REGN7650 | CD38x4-1BB (1 + 2), (hIgg4^{P-PVA}) |
| REGN9682 | CD38x4-1BB (1 + 2), (G4S)3 (hIgg4^{P-PVA}) |
| REGN9686 | CD38x4-1BB (1 + 2), (G4S)2 (hIgg4^{P-PVA}) |
| REGN4249 | Comparator 1 |
| REGN7540 | Isotype control (hIgg4^{P-PVA}) |
| REGN1945 | Isotype control (hIgg4^{P}) |

One day before the experiment, HEK293 reporter cells were split to 5×10⁵ cells/ml in DMEM+10% FBS+P/S/G+500 μg/ml G418 growth media.

On the day of the experiment adherent HEK293 reporter and target cells were trypsinized, washed, and resuspended in assay media (DMEM+10% FBS+P/S/G). The reporter HEK293/NFKB-Luc/h4-1BB cells were added to the wells of 96-well white microtiter plates at a final concentration of 5×10³ cells/well, followed by the addition of target cells, either HEK293/hCD20 or HEK293/hCD20/hCD38, added at a final concentration of 1×10⁴ cells/well or MOLP8 and OPM2 target cells added at a final concentration of 2.5×10⁴ cells/well.

CD38×4-1BB 1+2 and control antibodies were titrated in a 1:3, 10-point, serial dilution ranging from 3.0 pM to 20 nM final concentration, with the last point containing no antibody, included as a control. After addition of antibodies, the 96-well white microtiter plates were incubated at 37° C./5% CO₂ for 5 h followed by the addition of an equal volume of ONE-Glo™ (Promega) reagent to lyse cells and detect luciferase activity. The emitted light was captured in Relative Light Units (RLU) on a multi-label plate reader Envision (PerkinElmer). EC₅₀ values of the antibodies were determined from a 4-parameter logistic equation over a 10-point dose response curve (the 10^{th} point containing no antibody) using GraphPad Prism software. Results are provided in Table 18.

### HEK293/hCD20 & HEK293/hCD20/hCD38

In the presence of HEK293/hCD20/hCD38 target cells the CD38×4-1BB 1+2 bispecific antibodies (REGN7647, REGN9682, REGN9686, and REGN7650) led to a similar increase in luciferase activity, regardless of the linkage between the 2 anti-4-1BB binding Fab domains. The anti4-1BB bivalent antibody (REGN4249) also led to a dose dependent increase in luciferase activity, whereas the isotype control antibodies did not.

In the presence of HEK293/hCD20 target cells lacking CD38, no response was seen with the CD38×4-1BB bispecific antibodies nor with the isotype controls. Only the anti-4-1BB bivalent antibody (REGN4249) led to a dose dependent increase in luciferase activity.

### MOLP8 and OPM2 Cells

In the presence of either the MOLP8 or OPM2 target cells the CD38×4-1BB 1+2 (REGN7647, REGN7650, REGN9682 and REGN9686) antibodies as well as the bivalent anti-4-1BB antibody (REGN4249) led to a dose dependent increase in luciferase activity, with the different linker length 1+2 CD38×4-1BB variants resulting in similar activity. The isotype control antibodies did not result in any signal.

TABLE 18
Maximum Luciferase Activity and Potency Values of Antibodies

| | HEK293/CD20/CD38 | | HEK293/CD20 | | OPM2 | | MOLP-8 | |
|---|---|---|---|---|---|---|---|---|
| | MAX | EC50 | MAX | EC50 | MAX | EC50 | MAX | EC50 |
| | [RLU] | [M] | [RLU] | [M] | [RLU] | [M] | [RLU] | [M] |
| REGN7647 | 2.4E+06 | NC | 1.2E+06 | ND | 1.9E+06 | 7.35E−12 | 2.3E+06 | NC |
| REGN7650 | 2.3E+06 | NC | 1.5E+06 | NC | 2.0E+06 | 9.89E−11 | 2.4E+06 | NC |
| REGN9682 | 2.4E+06 | NC | 1.0E+06 | ND | 2.1E+06 | 2.19E−11 | 2.2E+06 | NC |
| REGN9686 | 2.4E+06 | NC | 1.1E+06 | NC | 2.1E+06 | 1.80E−11 | 2.2E+06 | NC |
| REGN4249 | 2.8E+06 | 4.28E−11 | 3.3E+06 | 6.82E−11 | 2.9E+06 | 5.25E−11 | 2.7E+06 | 4.59E−11 |
| REGN7540 | 9.9E+05 | ND | 1.2E+06 | ND | 8.5E+05 | ND | 7.9E+05 | ND |
| REGN1945 | 1.0E+06 | ND | 9.5E+05 | ND | 7.5E+05 | ND | 7.8E+05 | ND |

Abbreviations: ND: Not Determined, because a concentration-dependent increase was not observed; NC: Not calculated because the data did not fit a 4-parameter logistic equation.

### Example 10: Characterization of CD38×4-1BB 1+2 Bispecific Antibodies in T-Cell Allogeneic Cemiplimab Combination Assay Using NALM-6, NALM-6/PDL1, and Human Primary T-Cells

Two signals, “signal 1” & “signal 2”, are required for proper T cell activation. “Signal 1” is induced by binding of the T cell receptor (TCR) on T cells to peptide-bound major histocompatibility complex (MHC) molecules on antigen presenting cells (APCs). “Signal 2” is provided by engaging co-stimulatory receptors on T cells. One such costimulatory receptor is 4-11BB receptor, which is an inducible type I membrane protein and member of the tumor necrosis factor receptor (TNFR) superfamily. Expression of 4-1BB receptor is induced on the surface of T-cells after antigen- or mitogen-induced activation. The activation of 4-1BB occurs via engagement with 4-1BBL, present on APCs. Therefore, activation of 4-1BB signaling provides a targeted approach to enhance existing TCR signaling.

CD38×4-1BB (1+2) bispecific antibodies are designed to mimic the natural ligand of 4-11BB, by bridging CD38⁺ target cells with 4-1BB receptor positive T cells, to provide “signal 2” in order to enhance the activation of T cells in the presence of a “signal 1” provided by a Tumor-associated antigen (TAA)×CD3 bispecific antibody or an allogeneic response provided by the APC. However, T cell activation can be inhibited by the ligation of programmed cell death protein 1 receptor (PD-1) on T cells to its ligand PD-L1 on APCs. Ligated PD-1 leads to the recruitment of phosphatases to CD28 and the TCR complex (Zou et al. Inhibitory B7-family molecules in the tumor microenvironment. Nature Reviews Immunology 2008, 8:467-477; Francisco et al. 2010, Hui et al. T cell costimulatory receptor CD28 is a primary target for PD-1-mediated inhibition. Science. 2017, 355(6332):1428-33), which in turn counteract TCR signaling and 4-1BB stimulation. Thus, blockade of the PD-1/PD-L1 interaction with a PD-1 antagonist, cemiplimab, in combination with CD38×4-1BB bispecific antibodies may potentiate T cell function.

### Multispecific Antigen Binding Molecules:

Multispecific antigen binding molecules and the controls tested in this experiment are as shown in Table 19.

TABLE 19
CD38x4-1BB Binding Molecules and Controls

| MABM Identifier | Description |
|---|---|
| REGN7633 | CD38x4-1BB 1 + 2, hIgg4^{P-PVA} |
| REGN7647 | CD38x4-1BB 1 + 2, hIgg4^{P-PVA} |
| REGN7650 | CD38x4-1BB 1 + 2, hIgg4^{P-PVA} |
| REGN4249 | Comparator 1 |
| REGN2810 | PD-1 antibody (Cemiplimab), hIgg4^{P} |
| REGN7540 | Isotype control, hIgg4^{P-PVA} |
| REGN1945 | Isotype control, hIgg4^{P} |

The ability of CD38×4-1BB 1+2 multispecific antigen binding molecules to activate human primary T-cells by engaging CD38 and 4-1BB to deliver “signal 2”, as determined by IL-2 & IFNγ release, was evaluated in the presence of a CD38⁺ human acute lymphoblastic leukemia cancer cell line engineered to express PD-L1 (NALM6/hPD-L1). NALM6 cells provide an allogeneic TCR response sufficient to serve as “signal 1”. The addition of a fixed concentration of the PD-1 antagonist antibody, cemiplimab, was evaluated in the presence of a titration of CD38×4-1BB 1+2 or control antibodies.

### Isolation of Human Primary CD3⁺ T Cells:

Human peripheral blood mononuclear cells (PBMCs) were isolated from a healthy donor leukocyte pack from Precision for Medicine (Donor 555192) using the EasySep™ Direct Human PBMC Isolation Kit, following the manufacturers recommended protocol and frozen down. CD3⁺ T-cells were isolated by thawing vials of frozen PBMCs. Donor PBMCs were enriched for CD3⁺ T-cells using an EasySep™ Human CD3⁺ T Cell Isolation Kit from StemCell Technologies and following the manufacturer's recommended instructions.

### IL-2 & IFNγ Release Assay:

Enriched CD3⁺ T-cells, resuspended in stimulation media, were added into 96-well round bottom plates at a concentration of 1×10⁵ cells/well. NALM6 cells or NALM-6 cells engineered to express hPD-L1, were added to CD3⁺ T-cells at a final concentration of 5×10⁴ cells/well. Subsequently, REGN7633, REGN7647, REGN7650, REGN4249 and REGN7540, were titrated from 0.76 pM to 50 nM in a 1:4 dilution and added to wells. The final point of the 10-point dilution contained no titrated antibody. Following addition of titrated antibody, a constant 20 nM of either cemiplimab or its matched isotype control (REGN1945) was added to wells. Plates were incubated for 72 hours at 37° C., 5% CO₂ and 5 μL from supernatant was used for measuring IL-2 and IFNγ. The amount of cytokine in assay supernatant was determined using AlphaLisa kits from PerkinElmer following the manufacturer's protocol. The cytokine measurements were acquired on Perkin Elmer's multilabel plate reader Envision and values were reported as μg/mL. All serial dilutions were tested in triplicate.

### Results

The EC₅₀ values of the antibodies were determined from a four-parameter logistic equation over a 10-point dose-response curve using Graph Pad Prism™ software. Maximal cytokine is given as the mean max response detected within the tested dose range. Results are provided in Tables 20 and 21.

In the presence of allogeneic NALM6 cells or NALM6 cells engineered to express PD-L1, CD38×4-1BB 1+2 antibody treatment (REGN7633, REGN7647 and REGN7650), in comparison to matched isotype control (REGN7540), led to dose dependent increases in IL-2 and IFNγ release. The maximum IL-2 and IFNγ release was lower in conditions with NALM6/PD-L1 cells, compared to NALM-6 (not expressing PD-L). In the presence of NALM6 cells expressing PD-L1, cemiplimab increased maximum cytokine release in comparison to the matched isotype control for cemiplimab, REGN1945. Of note, 1+2 formats of CD38×4-1BB antibody led to higher maximum cytokine release and greater potency, compared to bivalent 4-1BB (REGN4249) antibody.

TABLE 20
Maximum IL-2 Release and Potency Values

| | NALM6 + | | NALM6 + | | NALM6/PD-L1 + | | NALM6/PD-L1 + | |
|---|---|---|---|---|---|---|---|---|
| | IgG4P | | Cemiplimab | | IgG4P | | Cemiplimab | |
| | MAX | EC50 | MAX | EC50 | MAX | EC50 | MAX | EC50 |
| IL-2 | [pg/ml] | [M] | [pg/ml] | [M] | [pg/ml] | [M] | [pg/ml] | [M] |
| REGN7633 | 853 | 6.22E−10 | 756 | 4.54E−10 | 205 | 4.13E−10 | 470 | 5.20E−10 |
| REGN7647 | 1117 | 5.79E−11 | 1043 | 4.77E−11 | 433 | 1.17E−10 | 683 | 9.11E−11 |
| REGN7650 | 1242 | 1.79E−10 | 1359 | 1.55E−10 | 326 | 2.28E−10 | 665 | 2.83E−10 |
| REGN7540 | 125 | ND | 135 | ND | 32 | ND | 102 | ND |
| REGN4249 | 413 | 6.03E−10 | 307 | 4.132E−10 | 143 | 3.96E−10 | 259 | 2.43E−10 |

Abbreviations: ND: Not Determined; NC: Not calculated because the data did not fit a 4-parameter logistic equation.

TABLE 21
Maximum IFNγ release and Potency Values

| | NALM-6 + | | NALM-6 + | | NALM-6/PD-L1 + | | NALM-6/PD-L1 + | |
|---|---|---|---|---|---|---|---|---|
| | IgG4P | | Cemiplimab | | IgG4P | | Cemiplimab | |
| | MAX | EC50 | MAX | EC50 | MAX | EC50 | MAX | EC50 |
| IFNY | [pg/ml] | [M] | [pg/ml] | [M] | [pg/ml] | [M] | [pg/ml] | [M] |
| REGN7633 | 519 | 6.37E−10 | 1095 | NC | 1 | ND | 272 | 1.28E−09 |
| REGN7647 | 707 | 8.15E−11 | 1067 | NC | 67 | NC | 557 | NC |
| REGN7650 | 1008 | NC | 962 | 1.56E−10 | 15 | NC | 244 | NC |
| REGN7540 | 86 | ND | 333 | ND | 1 | ND | 14 | ND |
| REGN4249 | 616 | NC | 919 | NC | 7 | NC | 324 | NC |

Abbreviations: ND: Not Determined; NC: Not calculated because the data did not fit a 4-parameter logistic equation.

### Example 11: In Vivo Anti-tumor Efficacy of CD38×4-1BB Bispecific Antibodies in Combination with a BCMA×CD3 Bispecific Antibody

Multispecific antigen binding molecules and the controls tested in this experiment are as shown in Table 22.

TABLE 22
CD38x4-1BB Binding Molecules and Controls

| MABM Identifier | Description |
|---|---|
| REGN5458 | BCMAxCD3 bsAB |
| REGN13168 | Non-TAAx4-1BB (1 + 2) Control, (hIgg4^{P-PVA}) |
| REGN9686 | CD38x4-1BB (1 + 2), (G4S)2 (hIgg4^{P-PVA}) |

To determine the in vivo anti-tumor efficacy of CD38×4-1BB 1+2 format bispecific antibodies (bsAb) in combination with a BCMA×CD3 bsAb, a xenogenic tumor study was performed. On day −11, immunodeficient NOD.Cg-Prkdc^{scid}II2rg^{tm1Wjl}/SzJ (NSG) mice were intraperitoneally injected with 4×10⁶ human peripheral blood mononuclear cells (PBMC) from a normal, healthy donor. On day 0, the mice were intravenously administered 2×10⁶ BCMA⁺CD38⁺MOLP-8 human multiple myeloma tumor cells that were engineered to also express firefly luciferase (MOLP-8-luciferase cells). The mice (n=4-5 per group) were then immediately administered either a CD3-binding control bispecific Ab or a BCMA×CD3 bispecific antibody (REGN5458; US Patent Application Publication 20200024356) at 0.4 mg/kg, in combination with a 4-1BB-binding control bispecific Ab (1+2 format) or a CD38×4-1BB bispecific antibody (1+2 format; REGN9686) at 4 mg/kg. The mice were administered these antibodies twice more on days 7 and 14, for a total of three doses. Tumor growth was assessed over 53 days by measuring tumor bioluminescence (BLI) in anesthetized animals. As a positive control, a group of mice (n=5) was given only MOLP-8-luciferase cells and PBMCs, but not antibody (PBS-treated group). In order to measure background BLI levels, a group of mice (n=5) were untreated and did not receive tumors, PBMC, or antibody (No Tumor group).

These studies demonstrate that while BCMA×CD3 bispecific antibody monotherapy demonstrates only modest anti-tumor efficacy and CD38×4-1BB 1+2 bispecific antibody monotherapy demonstrates little/no anti-tumor activity, combination treatment with BCMA×CD3 bispecific antibody plus CD38×4-1BB 1+2 bispecific antibody results in more potent, combinatorial anti-tumor efficacy that is superior to either therapy alone.

### Implantation and Measurement of Xenogenic Tumors

On day −11, immunodeficient NOD.Cg-Prkdc^{scid}II2rg^{tm1Wjl}/SzJ (NSG) mice were intraperitoneally injected with 4×10⁶ human peripheral blood mononuclear cells (PBMC) from a normal, healthy donor. On day 0, the mice were intravenously administered 2×10⁶ BCMA⁺CD38⁺MOLP-8 human multiple myeloma tumor cells that were engineered to also express firefly luciferase (MOLP-8-luciferase cells). The mice (n=4-5 per group) were then immediately administered either a CD3-binding control bispecific antibody or a BCMA×CD3 bispecific antibody (REGN5458) at 0.4 mg/kg, in combination with a 4-1BB-binding control bispecific antibody (1+2 format) or a CD38×4-1BB bispecific antibody (1+2 format; REGN9686) at 4 mg/kg. The mice were administered these Abs twice more on days 7 and 14, for a total of three doses. Tumor growth was assessed over 53 days by measuring tumor bioluminescence (BLI) in anesthetized animals. As a positive control, a group of mice (n=5) was given only MOLP-8-luciferase cells and PBMCs, but not antibody (PBS-treated group). In order to measure background BLI levels, a group of mice (n=5) were untreated and did not receive tumors, PBMC, or antibody (No Tumor group).

### Measurement of Xenogenic Tumor Growth

BLI imaging was used to measure tumor burden. Mice were injected IP with 150 mg/kg of the luciferase substrate D-luciferin suspended in PBS. Five minutes after this injection, BLI imaging of the mice was performed under isoflurane anesthesia using the Xenogen IVIS system. Image acquisition was carried out with the field of view at D, subject height of 1.5 cm, and medium binning level with automatic exposure time determined by the Living Image Software. BLI signals were extracted using Living Image software: regions of interest were drawn around each tumor mass and photon intensities were recorded as total flux (photons/second—p/s).

### Results:

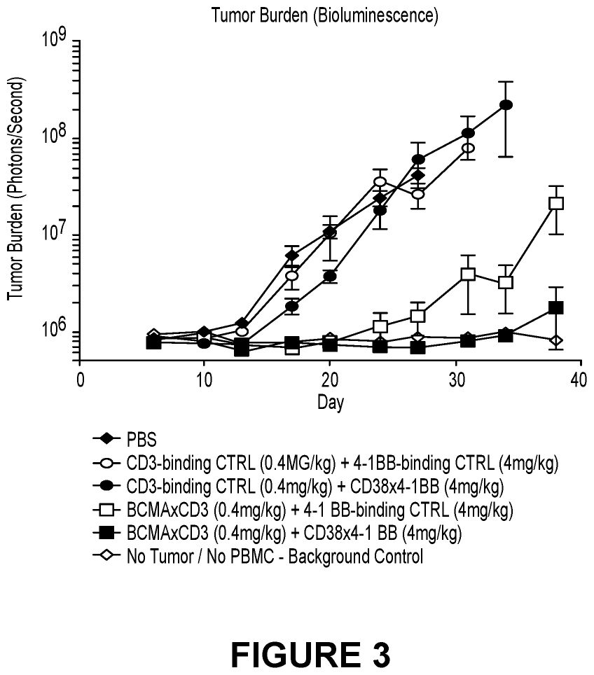

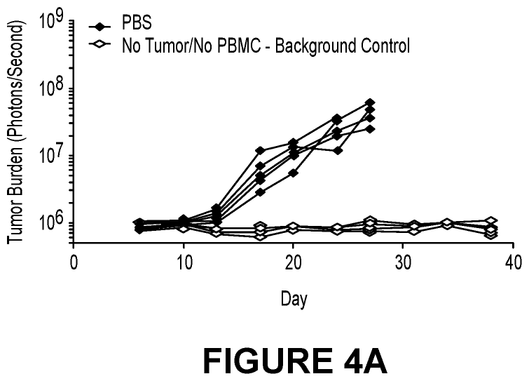

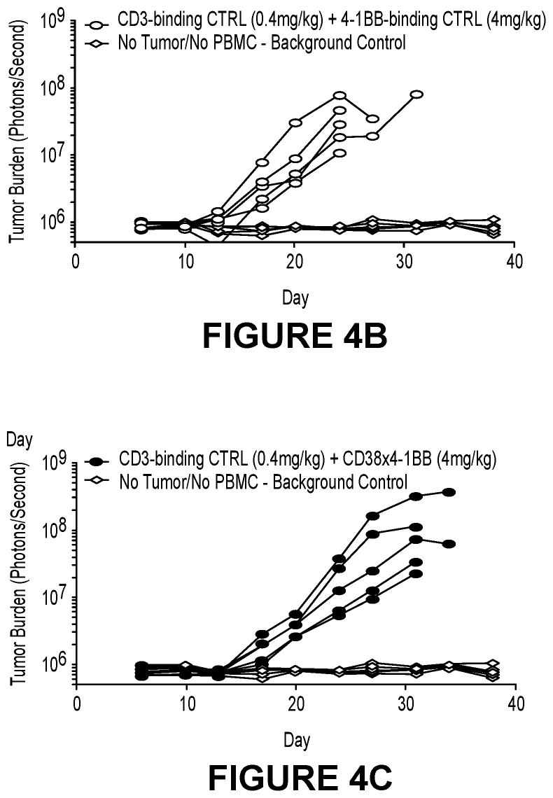

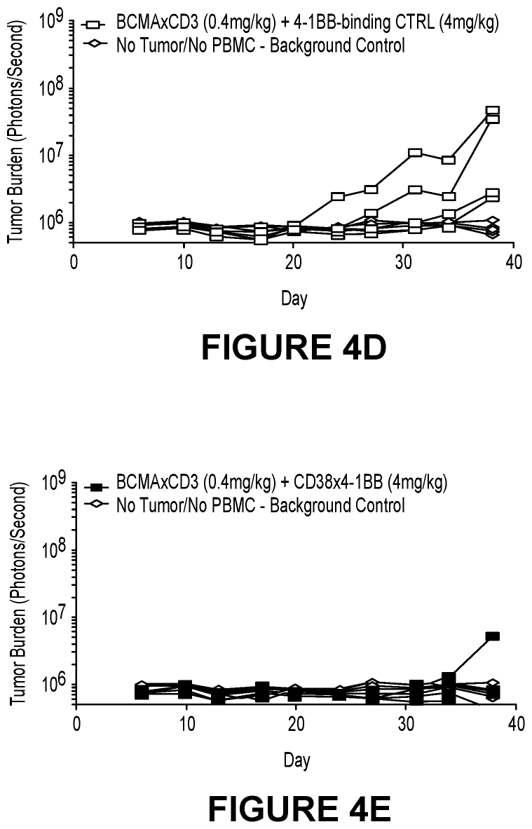

Tables 23-32 provide the results of the treatment combinations on tumor burden and subject survival at 6, 10, 13, 17, 20, 24, 27, 31, 34, and 38 days after administration of human multiple myeloma tumor cells. FIG. 3 is a graphical representation of the data shown in the tables over the 38 days. FIG. 4A illustrates tumor burden over time in the mice treated with PBS relative to mice that received no tumor cells; FIG. 4B illustrates tumor burden over time in the mice treated with CD3-binding control bsAb (0.4 mg/kg)+4-1BB-binding control bsAb (4 mg/kg) relative to mice that received no tumor cells; FIG. 4C illustrates tumor burden over time in the mice treated with CD3-binding control bsAb (0.4 mg/kg)+CD38×4-1BB (4 mg/kg) relative to mice that received no tumor cells; FIG. 4D illustrates tumor burden over time in the mice treated with BCMA×CD3 bsAb (0.4 mg/kg)+4-1BB-binding control bsAb (4 mg/kg) relative to mice that received no tumor cells; FIG. 4E illustrates tumor burden over time in the mice treated with BCMA×CD3 bsAb (0.4 mg/kg)+CD38×4-1BB (4 mg/kg) relative to mice that received no tumor cells.

BCMA×CD3 monotherapy: BCMA×CD3 bsAb (REGN5458) plus 4-1BB-binding control bsAb provided some anti-tumor efficacy, with mean BLI readings reduced compared to mice receiving CD3-binding control bsAb plus 4-1BB-binding control bsAb p<0.0001 on day 24 and p=0.0015 on day 26 by 2-way ANOVA analysis.

CD38×4-1BB monotherapy: Treatment with CD3-binding control bsAb plus CD38×4-1BB 1+2 bsAb (REGN9686) did not significantly reduce mean BLI readings compared to mice receiving CD3-binding control bsAb plus 4-1BB-binding control bsAb.

BCMA×CD3+CD38×4-1BB 1+2: The combination of BCMA×CD3 bsAb (REGN5458) plus CD38×4-1BB 1+2 bsAb (REGN9686) resulted in mean BLI readings that were lower than mice receiving BCMA×CD3 bsAb plus 4-1BB-binding control bsAb (p<0.0001 on day 38 by 2-way ANOVA analysis).

Thus, these studies demonstrate that while BCMA×CD3 bsAb monotherapy demonstrates only modest anti-tumor efficacy and CD38×4-1BB 1+2 bsAb monotherapy demonstrates little/no anti-tumor activity, combination treatment with BCMA×CD3 bsAb plus CD38×4-1BB 1+2 bsAb results in more potent, combinatorial anti-tumor efficacy that is superior to either therapy alone.

TABLE 23
Anti-Tumor Efficacy Through Combination Treatment with
BCMAxCD3 bsAb Plus CD38x4-1BB 1 + 2 bsAb - Day 6

| | Tumor | | Number |
|---|---|---|---|
| | Burden - | | of mice |
| | Mean Total | Total Flux | still |
| Antibody | Flux (p/s) | SEM on | alive on |
| Treatment | on Day 6 | Day 6 | day 6 |
| PBS vehicle | 8.67E+05 | 4.66E+04 | 5 |
| CD3-binding control bsAb | 8.80E+05 | 4.38E+04 | 5 |
| (0.4 mg/kg) + 4-1BB-binding | | | |
| control bsAb (4 mg/kg) | | | |
| CD3-binding control bsAb | 7.81E+05 | 5.36E+04 | 5 |
| (0.4 mg/kg) + CD38x4-1BB | | | |
| (4 mg/kg) | | | |
| BCMAxCD3 bsAb (0.4 mg/kg) + | 8.40E+05 | 4.47E+04 | 4 |
| 4-1BB-binding control bsAb | | | |
| (4 mg/kg) | | | |
| BCMAxCD3 bsAb (0.4 mg/kg) + | 7.82E+05 | 2.34E+04 | 5 |
| CD38x4-1BB (4 mg/kg) | | | |
| No Tumor (Background BLI) | 9.54E+05 | 3.42E+04 | 5 |

TABLE 24
Anti-Tumor Efficacy Through Combination Treatment with
BCMAxCD3 bsAb Plus CD38x4-1BB 1 + 2 bsAb - Day 10

| | Tumor | | Number |
|---|---|---|---|
| | Burden - | Total | of mice |
| | Mean Total | Flux | still |
| Antibody | Flux (p/s) | SEM on | alive on |
| Treatment | on Day 10 | Day 10 | day 10 |
| PBS vehicle | 1.01E+06 | 4.01E+04 | 5 |
| CD3-binding control bsAb | 8.84E+05 | 2.98E+04 | 5 |
| (0.4 mg/kg) + 4-1BB-binding | | | |
| control bsAb (4 mg/kg) | | | |
| CD3-binding control bsAb | 7.69E+05 | 3.04E+04 | 5 |
| (0.4 mg/kg) + CD38x4-1BB | | | |
| (4 mg/kg) | | | |
| BCMAxCD3 bsAb (0.4 mg/kg) + | 8.96E+05 | 4.41E+04 | 4 |
| 4-1BB-binding control bsAb | | | |
| (4 mg/kg) | | | |
| BCMAxCD3 bsAb (0.4 mg/kg) + | 8.49E+05 | 4.95E+04 | 5 |
| CD38x4-1BB (4 mg/kg) | | | |
| No Tumor (Background BLI) | 9.41E+05 | 3.62E+04 | 5 |

TABLE 25
Anti-Tumor Efficacy Through Combination Treatment with
BCMAxCD3 bsAb Plus CD38x4-1BB 1 + 2 bsAb - Day 13

| | Tumor | | Number |
|---|---|---|---|
| | Burden - | Total | of mice |
| | Mean Total | Flux | still |
| Antibody | Flux (p/s) | SEM on | alive on |
| Treatment | on Day 13 | Day 13 | day 13 |
| PBS vehicle | 1.24E+06 | 1.07E+05 | 5 |
| CD3-binding control bsAb | 1.01E+06 | 1.60E+05 | 5 |
| (0.4 mg/kg) + 4-1BB-binding | | | |
| control bsAb (4 mg/kg) | | | |
| CD3-binding control bsAb | 7.67E+05 | 2.92E+04 | 5 |
| (0.4 mg/kg) + CD38x4-1BB | | | |
| (4 mg/kg) | | | |
| BCMAxCD3 bsAb (0.4 mg/kg) + | 7.20E+05 | 3.41E+04 | 4 |
| 4-1BB-binding control bsAb | | | |
| (4 mg/kg) | | | |
| BCMAxCD3 bsAb (0.4 mg/kg) + | 6.37E+05 | 2.39E+04 | 5 |
| CD38x4-1BB (4 mg/kg) | | | |
| No Tumor (Background BLI) | 7.88E+05 | 3.52E+04 | 5 |

TABLE 26
Anti-Tumor Efficacy Through Combination Treatment with
BCMAxCD3 bsAb Plus CD38x4-1BB 1 + 2 bsAb - Day 17

| | Tumor | | Number |
|---|---|---|---|
| | Burden - | Total | of mice |
| | Mean Total | Flux | still |
| Antibody | Flux (p/s) | SEM on | alive on |
| Treatment | on Day 17 | Day 17 | day 17 |
| PBS vehicle | 6.16E+06 | 1.51E+06 | 5 |
| CD3-binding control bsAb | 3.81E+06 | 1.07E+06 | 5 |
| (0.4 mg/kg) + 4-1BB-binding | | | |
| control bsAb (4 mg/kg) | | | |
| CD3-binding control bsAb | 1.84E+06 | 3.30E+05 | 5 |
| (0.4 mg/kg) + CD38x4-1BB | | | |
| (4 mg/kg) | | | |
| BCMAxCD3 bsAb (0.4 mg/kg) + | 6.74E+05 | 7.70E+04 | 4 |
| 4-1 BB-binding control bsAb | | | |
| (4 mg/kg) | | | |
| BCMAxCD3 bsAb (0.4 mg/kg) + | 7.69E+05 | 5.96E+04 | 5 |
| CD38x4-1BB (4 mg/kg) | | | |
| No Tumor (Background BLI) | 7.63E+05 | 4.76E+04 | 5 |

TABLE 27
Anti-Tumor Efficacy Through Combination Treatment with
BCMAxCD3 bsAb Plus CD38x4-1BB 1 + 2 bsAb - Day 20

| | Tumor | | Number |
|---|---|---|---|
| | Burden - | Total | of mice |
| | Mean Total | Flux | still |
| Antibody | Flux (p/s) | SEM on | alive on |
| Treatment | on Day 20 | Day 20 | day 20 |
| PBS vehicle | 1.10E+07 | 1.70E+06 | 5 |
| CD3-binding control bsAb | 1.06E+07 | 5.11E+06 | 5 |
| (0.4 mg/kg) + 4-1BB-binding | | | |
| control bsAb (4 mg/kg) | | | |
| CD3-binding control bsAb | 3.81E+06 | 5.67E+05 | 5 |
| (0.4 mg/kg) + CD38x4-1BB | | | |
| (4 mg/kg) | | | |
| BCMAxCD3 bsAb (0.4 mg/kg) + | 7.88E+05 | 3.78E+04 | 4 |
| 4-1BB-binding control bsAb | | | |
| (4 mg/kg) | | | |
| BCMAxCD3 bsAb (0.4 mg/kg) + | 7.36E+05 | 2.41E+04 | 5 |
| CD38x4-1BB (4 mg/kg) | | | |
| No Tumor (Background BLI) | 8.52E+05 | 1.57E+04 | 5 |

TABLE 28
Anti-Tumor Efficacy Through Combination Treatment with
BCMAxCD3 bsAb Plus CD38x4-1BB 1 + 2 bsAb - Day 24

| | Tumor | | Number |
|---|---|---|---|
| | Burden - | Total | of mice |
| | Mean Total | Flux | still |
| Antibody | Flux (p/s) | SEM on | alive on |
| Treatment | on Day 24 | Day 24 | day 24 |
| PBS vehicle | 2.44E+07 | 4.43E+06 | 5 |
| CD3-binding control bsAb | 3.60E+07 | 1.18E+07 | 5 |
| (0.4 mg/kg) + 4-1BB-binding | | | |
| control bsAb (4 mg/kg) | | | |
| CD3-binding control bsAb | 1.82E+07 | 6.39E+06 | 5 |
| (0.4 mg/kg) + CD38x4-1BB | | | |
| (4 mg/kg) | | | |
| BCMAxCD3 bsAb (0.4 mg/kg) + | 1.14E+06 | 4.30E+05 | 4 |
| 4-1BB-binding control bsAb | | | |
| (4 mg/kg) | | | |
| BCMAxCD3 bsAb (0.4 mg/kg) + | 7.15E+05 | 1.19E+04 | 5 |
| CD38x4-1BB (4 mg/kg) | | | |
| No Tumor (Background BLI) | 7.91E+05 | 2.05E+04 | 5 |

TABLE 29
Anti-Tumor Efficacy Through Combination Treatment with
BCMAxCD3 bsAb Plus CD38x4-1BB 1 + 2 bsAb - Day 27

| | Tumor | | Number |
|---|---|---|---|
| | Burden - | Total | of mice |
| | Mean Total | Flux | still |
| Antibody | Flux (p/s) | SEM on | alive on |
| Treatment | on Day 27 | Day 27 | day 27 |
| PBS vehicle | 4.21E+07 | 7.71E+06 | 4 |
| CD3-binding control bsAb | 2.67E+07 | 7.65E+06 | 2 |
| (0.4 mg/kg) + 4-1BB-binding | | | |
| control bsAb (4 mg/kg) | | | |
| CD3-binding control bsAb | 6.11E+07 | 3.02E+07 | 5 |
| (0.4 mg/kg) + CD38x4-1BB | | | |
| (4 mg/kg) | | | |
| BCMAxCD3 bsAb (0.4 mg/kg) + | 1.47E+06 | 5.55E+05 | 4 |
| 4-1BB-binding control bsAb | | | |
| (4 mg/kg) | | | |
| BCMAxCD3 bsAb (0.4 mg/kg) + | 6.92E+05 | 5.29E+04 | 5 |
| CD38x4-1BB (4 mg/kg) | | | |
| No Tumor (Background BLI) | 8.85E+05 | 6.27E+04 | 5 |

TABLE 30
Anti-Tumor Efficacy Through Combination Treatment with
BCMAxCD3 bsAb Plus CD38x4-1BB 1 + 2 bsAb - Day 31

| | Tumor | | Number |
|---|---|---|---|
| | Burden - | Total | of mice |
| | Mean Total | Flux | still |
| Antibody | Flux (p/s) | SEM on | alive on |
| Treatment | on Day 31 | Day 31 | day 31 |
| PBS vehicle | No mice alive | N/A | 0 |
| CD3-binding control bsAb | 7.95E+07 | 0.00E+00 | 1 |
| (0.4 mg/kg) + 4-1 BB-binding | | | |
| control bsAb (4 mg/kg) | | | |
| CD3-binding control bsAb | 1.15E+08 | 5.50E+07 | 5 |
| (0.4 mg/kg) + CD38x4-1BB | | | |
| (4 mg/kg) | | | |
| BCMAxCD3 bsAb (0.4 mg/kg) + | 3.91E+06 | 2.35E+06 | 4 |
| 4-1 BB-binding control bsAb | | | |
| (4 mg/kg) | | | |
| BCMAxCD3 bsAb (0.4 mg/kg) + | 8.08E+05 | 8.44E+04 | 5 |
| CD38x4-1BB (4 mg/kg) | | | |
| No Tumor (Background BLI) | 8.65E+05 | 3.41E+04 | 5 |

TABLE 31
Anti-Tumor Efficacy Through Combination Treatment with
BCMAxCD3 bsAb Plus CD38x4-1BB 1 + 2 bsAb - Day 34

| | Tumor | | Number |
|---|---|---|---|
| | Burden - | Total | of mice |
| | Mean Total | Flux | still |
| Antibody | Flux (p/s) | SEM on | alive on |
| Treatment | on Day 34 | Day 34 | day 34 |
| PBS vehicle | No mice alive | N/A | 0 |
| CD3-binding control bsAb | No mice alive | N/A | 0 |
| (0.4 mg/kg) + 4-1 BB-binding | | | |
| control bsAb (4 mg/kg) | | | |
| CD3-binding control bsAb | 2.24E+08 | 1.60E+08 | 2 |
| (0.4 mg/kg) + CD38x4-1BB | | | |
| (4 mg/kg) | | | |
| BCMAxCD3 bsAb (0.4 mg/kg) + | 3.25E+06 | 1.75E+06 | 4 |
| 4-1 BB-binding control bsAb | | | |
| (4 mg/kg) | | | |
| BCMAxCD3 bsAb (0.4 mg/kg) + | 9.27E+05 | 1.26E+05 | 5 |
| CD38x4-1BB (4 mg/kg) | | | |
| No Tumor (Background BLI) | 9.75E+05 | 2.55E+04 | 5 |

TABLE 32
Anti-Tumor Efficacy Through Combination Treatment with
BCMAxCD3 bsAb Plus CD38x4-1BB 1 + 2 bsAb - Day 38

| | Tumor | | Number |
|---|---|---|---|
| | Burden - | Total | of mice |
| | Mean Total | Flux | still |
| Antibody | Flux (p/s) | SEM on | alive on |
| Treatment | on Day 38 | Day 38 | day 38 |
| PBS vehicle | No mice alive | N/A | 0 |
| CD3-binding control bsAb | No mice alive | N/A | 0 |
| (0.4 mg/kg) + 4-1BB-binding | | | |
| control bsAb (4 mg/kg) | | | |
| CD3-binding control bsAb | No mice alive | N/A | 0 |
| (0.4 mg/kg) + CD38x4-1BB | | | |
| (4 mg/kg) | | | |
| BCMAxCD3 bsAb (0.4 mg/kg) + | 2.12E+07 | 1.11E+07 | 4 |
| 4-1BB-binding control bsAb | | | |
| (4 mg/kg) | | | |
| BCMAxCD3 bsAb (0.4 mg/kg) + | 1.79E+06 | 1.13E+06 | 4 |
| CD38x4-1BB (4 mg/kg) | | | |
| No Tumor (Background BLI) | 8.22E+05 | 7.30E+04 | 5 |

### Example 12: Biacore Binding Kinetics of Anti-CD38×4-1BB Antigen-Binding Molecules

Binding kinetics of the anti-CD38×4-1BB antibodies were determined by antibody capture format Biacore binding kinetics of anti-CD38×4-1BB 1+2 antibodies binding to monomeric and dimeric human 4-1BB reagents and antigen capture format Biacore binding kinetics of anti CD38×4-1BB 1+2 antibodies binding to dimeric human 4-1BB reagent. Both experiments were performed at 25° C.

### Antibody Capture Format Method:

Equilibrium dissociation constants (K_{D} values) for human 4-1BB expressed with a C-terminal myc-myc-hexahistidine tag (h4-1BB.mmH, REGN3584) or human 4-1BB expressed with a C-terminal murine Fc tag (h4-1BB.mFc, REGN3585) binding to purified anti-CD38×4-1BB 1+2 antibodies were determined using a real-time surface plasmon resonance biosensor using a Biacore 8k instrument. The CM5 Biacore sensor surface was derivatized by amine coupling with a monoclonal mouse anti-human Fc antibody (REGN2567). All Biacore binding studies were performed in a buffer composed of 0.01M HEPES pH 7.4, 0.15M NaCl, 3 mM EDTA, 0.05% v/v Surfactant P20 (HBS-EP running buffer). Different concentrations of h4-1BB.mmH (REGN3584) prepared in HBS-EP running buffer (ranging from 100 to 6.25 nM in 4-fold serial dilutions) or h4-1BB.mFc (REGN3585) prepared in HBS-EP running buffer (ranging from 100 to 1.56 nM in 4-fold serial dilutions) were injected over the captured anti-CD38×4-1BB 1+2 antibodies at a flow rate of 30 μL/minute. Antibody-reagent association was monitored for 5 minutes while dissociation in HBS-EP running buffer was monitored for 10 minutes. At the end of each cycle, the anti-CD38×4-1BB 1+2 antibody capture surface was regenerated using a 10 second injection of 20 mM phosphoric acid. All binding kinetics experiments were performed at 25° C. Results are presented in Tables 33 and 34.

### Antigen Capture Format Method:

Equilibrium dissociation constants (K_{D} values) for human 4-1BB expressed with a C-terminal murine Fc tag (h4-1BB.mFc, REGN3585) binding to purified anti-CD38×4-1BB 1+2 antibodies were determined using a real-time surface plasmon resonance biosensor using a Biacore 4000 instrument. The CM5 Biacore sensor surface was derivatized by amine coupling with a polyclonal rabbit anti-mouse Fc antibody (GE, #BR-1008-38). All Biacore binding studies were performed in a buffer composed of 0.01M HEPES pH 7.4, 0.15M NaCl, 3 mM EDTA, 0.05% v/v Surfactant P20 (HBS-EP running buffer). Different concentrations of CD38×4-1BB 1+2 constructs prepared in HBS-EP running buffer (ranging from 100 to 6.25 nM in 4-fold serial dilutions) were injected over the h4-1BB.mFc (REGN3585) captured surface at a flow rate of 30 μL/minute. Antibody-reagent association was monitored for 5 minutes while dissociation in HBS-EP running buffer was monitored for 10 minutes. At the end of each cycle, the h4-1BB.mFc capture surface was regenerated using a 40 sec injection of 10 mM glycine, pH 1.5. All binding kinetics experiments were performed at 25° C. Results are presented in Table 35.

### Data Analysis of Both Formats:

Kinetic association (kₐ) and dissociation (k_{d}) rate constants were determined by fitting the real-time sensorgrams to a 1:1 binding model using Cytiva Insight curve fitting software. Binding dissociation equilibrium constants (K_{D}) and dissociative half-lives (t/2) were calculated from the kinetic rate constants as:

$$\begin{matrix}{K}_{D} ( M ) = \frac{k d}{k a} , & and & t 1 / 2 ( min ) = \frac{ln ( 2 )}{60 * k d}\end{matrix}$$

TABLE 33
Kinetic and Equilibrium Binding Parameters of Monomeric Human 4-1BB to Surface-
captured anti-CD38x4-1BB 1 + 2 Antibodies at 25° C.

| | Ab | 100 nM h4- | | | | |
|---|---|---|---|---|---|---|
| REGN #/ | Capture | 1BB.mmH Bind | ka | kd | KD | t½ |
| Ab PID # | (RU) | (RU) | (1/Ms) | (1/s) | (M) | (min) |
| REGN7633 | 394.6 ± 1.4 | 24.9* | IC** | IC** | IC** | IC** |
| REGN7647 | 506.8 ± 3.1 | 86.1 | 3.24E+05 | 5.33E−03 | 1.65E−08 | 2.2 |
| REGN7650 | 458.9 ± 0.4 | 41.9 | 2.62E+05 | 5.35E−03 | 2.04E−08 | 2.2 |
| REGN9682 | 607.1 ± 1.1 | 106.8 | 3.58E+05 | 4.94E−03 | 1.38E−08 | 2.3 |
| REGN9686 | 606.9 ± 0.5 | 101.3 | 3.19E+05 | 4.74E−03 | 1.48E−08 | 2.4 |

*Dose-dependent binding of h4-1bb.mmH was observed
**IC: Observed binding did not fit to the binding simulation model and no binding kinetic parameters were determined under the current experimental conditions.

TABLE 34
Kinetic and Equilibrium Binding Parameters of Dimeric Human 4-1BB to Surface-
captured anti-CD38x4-1BB 1 + 2 Antibodies at 25° C.

| | Ab | 100 nM h4- | | | | |
|---|---|---|---|---|---|---|
| REGN #/ | Capture | 1BB.mFc Bind | ka | kd | KD | t½ |
| Ab PID # | (RU) | (RU) | (1/Ms) | (1/s) | (M) | (min) |
| REGN7633 | 395.4 ± 0.3 | 82.1 | 2.74E+05 | 2.51E−04 | 9.17E−10 | 46.0 |
| REGN7647 | 505.3 ± 1.4 | 223.6 | 1.95E+05 | 2.31E−04 | 1.18E−09 | 50.0 |
| REGN7650 | 458.3 ± 0.6 | 152.0 | 1.02E+05 | 1.22E−04 | 1.20E−09 | 94.9 |
| REGN9682 | 603.2 ± 1.7 | 283.3 | 2.06E+05 | 1.77E−04 | 8.60E−10 | 65.3 |
| REGN9686 | 600.2 ± 2.4 | 267.4 | 1.71E+05 | 2.05E−04 | 1.20E−09 | 56.3 |

TABLE 35
Kinetic and Equilibrium Binding Parameters of anti-CD38x4-1BB 1 + 2
Antibodies to Surface-captured Dimeric Human 4-1BB at 25° C.

| | | 100 nM Ab | | | | |
|---|---|---|---|---|---|---|
| REGN #/ | Ag+ | Bind | ka | kd | KD | t½ |
| Ab PID # | Capture | (RU) | (1/Ms) | (1/s) | (M) | (min) |
| REGN7633 | 132.0 ± 0.4 | 285.3 | 1.86E+05 | 2.37E−03 | 1.27E−08 | 4.9 |
| REGN7647 | 142.2 ± 0.5 | 205.8 | 6.26E+05 | 3.51E−04 | 5.60E−10 | 32.9 |
| REGN7650 | 127.5 ± 0.8 | 265.7 | 2.12E+05 | 1.69E−04 | 7.97E−10 | 68.4 |
| REGN9682 | 116.8 ± 0.4 | 233.7 | 4.30E+05 | 3.08E−04 | 7.16E−10 | 37.6 |
| REGN9686 | 127.4 ± 0.8 | 232.3 | 4.70E+05 | 3.37E−04 | 7.17E−10 | 34.3 |

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A bispecific antigen-binding molecule comprising:
(a) a first antigen-binding arm comprising three complementarity determining regions (CDRs) of a heavy chain variable region (HCVR) and three CDRs of a LCVR, wherein the first antigen-binding arm binds specifically to CD38, wherein the first antigen-binding arm comprises three CDRs of a HCVR comprising an amino acid sequence selected from the group consisting of SEQ ID NOs: 2 and 40 and three CDRs of a LCVR comprising an amino acid sequence selected from the group consisting of SEQ ID NOs: 18 and 48; and
(b) a second antigen-binding arm comprising a first antigen-binding region (R1) comprising three CDRs of a HCVR (R1-HCVR) and three CDRs of a LCVR (R1-LCVR); and a second antigen-binding region (R2) comprising three CDRs of a HCVR (R2-HCVR) and three CDRs of a LCVR (R2-LCVR), wherein the second antigen-binding arm binds specifically to 4-1BB.

2. The bispecific antigen-binding molecule of claim 1, wherein R1 and R2 bind to the same epitope on 4-1BB.

3. The bispecific antigen-binding molecule of claim 1, wherein R1 and R2 bind to different epitopes on 4-1BB.

4. The bispecific antigen-binding molecule of claim 1, wherein R1 and R2 are connected via a peptide linker.

5. The bispecific antigen-binding molecule of claim 4, wherein the peptide linker comprises a peptide sequence of (GGGGS)n, wherein n is 1 to 6.

6. (canceled)

7. (canceled)

8. The bispecific antigen-binding molecule of claim 1, wherein the first antigen-binding arm comprises three heavy chain complementarity determining regions (HCDR1-HCDR2-HCDR3) comprising an amino acid sequence selected from the group consisting of SEQ ID NOs: 4-6-8, and 42-44-46, respectively.

9. The bispecific antigen-binding molecule of claim 1, wherein the first antigen-binding arm comprises three light chain complementarity determining regions (LCDR1-LCDR2-LCDR3) comprising an amino acid sequence selected from the group consisting of SEQ ID NOs: 20-22-24 and 50-52-54, respectively.

10. The bispecific antigen-binding molecule of claim 1, wherein the first antigen-binding arm comprises a HCVR comprising the amino acid sequence selected from the group consisting of SEQ ID NOs: 2 and 40.

11. The bispecific antigen-binding molecule of claim 1, wherein the first antigen-binding arm comprises a LCVR comprising the amino acid sequence selected from the group consisting of SEQ ID NOs: 18 and 48.

12. The bispecific antigen-binding molecule of claim 1, wherein the first antigen-binding arm comprises a HCVR comprising the amino acid sequence selected from the group consisting of SEQ ID NOs: 2 and 40; and a LCVR comprising the amino acid sequence selected from the group consisting of SEQ ID NOs: 18 and 48.

13. The bispecific antigen-binding molecule of claim 1, wherein R1 comprises three CDRs of a HCVR (R1-HCVR) comprising an amino acid sequence selected from the group consisting of SEQ ID NOs: 10, 32, 62, 72, 86 and 94.

14. The bispecific antigen-binding molecule of claim 1, wherein R1 comprises three CDRs of a LCVR (R1-LCVR) comprising an amino acid sequence selected from the group consisting of SEQ ID NOs: 18 and 48.

15. The bispecific antigen-binding molecule of claim 1, wherein R1 comprises three heavy chain complementarity determining regions (R1-HCDR1-R1-HCDR2-R1-HCDR3) comprising an amino acid sequence selected from the group consisting of SEQ ID NOs: 12-14-16, 34-36-38, 64-66-68, 74-76-78, 88-90-92, and 96-98-100, respectively.

16. The bispecific antigen-binding molecule claim 1, wherein R1 comprises three light chain complementarity determining regions (R1-LCDR1-R1-LCDR2-R1-LCDR3) comprising an amino acid sequence selected from the group consisting of SEQ ID NOs: 20-22-24 and 50-52-54, respectively.

17. The bispecific antigen-binding molecule of claim 1, wherein R1 comprises a HCVR (R1-HCVR) comprising the amino acid sequence selected from the group consisting of SEQ ID NOs: 10, 32, 62, 72, 86 and 94.

18. The bispecific antigen-binding molecule of claim 1, wherein R1 comprises a LCVR (R1-LCVR) comprising the amino acid sequence selected from the group consisting of SEQ ID NOs: 18 and 48.

19. The bispecific antigen-binding molecule of claim 1, wherein R1 comprises a R1-HCVR comprising the amino acid sequence selected from the group consisting of SEQ ID NOs: 10, 32, 62, 72, 86 and 94; and a R1-LCVR comprising the amino acid sequence selected from the group consisting of SEQ ID NOs: 18 and 48.

20. The bispecific antigen-binding molecule of claim 1, wherein R2 comprises three CDRs of a HCVR (R2-HCVR) comprising the amino acid sequence selected from the group consisting of SEQ ID NOs: 10, 32, 62, 72, 86 and 94.

21. The bispecific antigen-binding molecule of claim 1, wherein R2 comprises three CDRs of a LCVR (R2-LCVR) comprising the amino acid sequence selected from the group consisting of SEQ ID NOs: 18 and 48.

22. The bispecific antigen-binding molecule claim 1, wherein R2 comprises three heavy chain complementarity determining regions (R2-HCDR1-R2-HCDR2-R2-HCDR3) comprising an amino acid sequence selected from the group consisting of SEQ ID NOs: 12-14-16, 34-36-38, 64-66-68, 74-76-78, 88-90-92, and 96-98-100, respectively.

23. The bispecific antigen-binding molecule of claim 1, wherein R2 comprises three light chain complementarity determining regions (R2-LCDR1-R2-LCDR2-R2-LCDR3) comprising an amino acid sequence selected from the group consisting of SEQ ID NOs: 20-22-24 and 50-52-54, respectively.

24. The bispecific antigen-binding molecule of claim 1, wherein R2 comprises a HCVR (R2-HCVR) comprising the amino acid sequence selected from the group consisting of SEQ ID NOs: 10, 32, 62, 72, 86 and 94.

25. The bispecific antigen-binding molecule of claim 1, wherein R2 comprises a LCVR (R2-LCVR) comprising the amino acid sequence selected from the group consisting of SEQ ID NOs: 18 and 48.

26. The bispecific antigen-binding molecule of claim 1, wherein R2 comprises a R2-HCVR comprising the amino acid sequence selected from the group consisting of SEQ ID NOs: 10, 32, 62, 72, 86 and 94; and a R2-LCVR comprising the amino acid sequence selected from the group consisting of SEQ ID NOs: 18 and 48.

27. The bispecific antigen-binding molecule of claim 1, wherein:
(a) the first antigen-binding arm comprises three CDRs of a HCVR comprising the amino acid sequence of SEQ ID NO: 40, and three CDRs of a LCVR comprising the amino acid sequence of SEQ ID NO: 48; and
(b) the second antigen-binding arm comprises:
(i) a first antigen-binding region (R1) comprising three CDRs of R1-HCVR comprising the amino acid sequence selected from the group consisting of SEQ ID NOs: 32, 62 and 72; and three CDRs of R1-LCVR comprising the amino acid sequence of SEQ ID NO: 48; and
(ii) a second antigen-binding region (R2) comprising three CDRs of R2-HCVR comprising the amino acid sequence selected from the group consisting of SEQ ID NOs: 62, 86, and 94; and three CDRs of R2-LCVR comprising the amino acid sequence of SEQ ID No: 48.

28. The bispecific antigen-binding molecule of claim 1, wherein:
(a) the first antigen-binding arm comprises HCDR1-HCDR2-HCDR3-LCDR1-LCDR2-LCDR3 comprising amino acid sequences selected from the group consisting of SEQ ID NOs: 42-44-46-50-52-54, respectively; and
(b) the second antigen-binding arm comprises:
(i) a first antigen-binding region (R1) comprising R1-HCDR1-R1-HCDR2-R1-HCDR3-R1-LCDR1-R1-LCDR2-R1-LCDR3 comprising amino acid sequences selected from the group consisting of SEQ ID NOs: 34-36-38-50-52-54, 64-66-68-50-52-54, and 74-76-78-50-52-54, respectively; and
(ii) a second antigen-binding region (R2) comprising R2-HCDR1-R2-HCDR2-R2-HCDR3-R2-LCDR1-R2-LCDR2-R2-LCDR3 comprising amino acid sequences selected from the group consisting of SEQ ID NOs: 64-66-68-50-52-54, 74-76-78-50-52-54, and 88-90-92-50-52-54, respectively.

29. The bispecific antigen-binding molecule of claim 1, wherein:
(a) the first antigen-binding arm comprises a HCVR comprising the amino acid sequence of SEQ ID NO: 40 and a LCVR comprising the amino acid sequence of SEQ ID NO: 48; and
(b) the second antigen-binding arm comprises:
(i) a first antigen-binding region (R1) comprising R1-HCVR comprising the amino acid sequence selected from the group consisting of SEQ ID NOs: 32, 62 and 72; and R1-LCVR comprising the amino acid sequence of SEQ ID NO: 48; and
(ii) a second antigen-binding region (R2) comprising R2-HCVR comprising the amino acid sequence selected from the group consisting of SEQ ID NOs: 62, 86 and 94; and R2-LCVR comprising the amino acid sequence of SEQ ID NO: 48.

30. The bispecific antigen-binding molecule of claim 1, wherein:
(a) the first antigen-binding arm comprises a HCVR comprising the amino acid sequence of SEQ ID NO: 40 and a LCVR comprising the amino acid sequence of SEQ ID NO: 48; and
(b) the second antigen-binding arm comprises:
(i) a first antigen-binding region (R1) comprising R1-HCVR comprising the amino acid sequence of SEQ ID NO: 32; and R1-LCVR comprising the amino acid sequence of SEQ ID NO: 48; and
(ii) a second antigen-binding region (R2) comprising R2-HCVR comprising the amino acid sequence of SEQ ID NO: 86; and R2-LCVR comprising the amino acid sequence of SEQ ID NO: 48.

31. The bispecific antigen-binding molecule of claim 1, wherein:
(a) the first antigen-binding arm comprises a HCVR comprising the amino acid sequence of SEQ ID NO: 40 and a LCVR comprising the amino acid sequence of SEQ ID NO: 48; and
(b) the second antigen-binding arm comprises:
(i) a first antigen-binding region (R1) comprising R1-HCVR comprising the amino acid sequence of SEQ ID NOs: 72; and R1-LCVR comprising the amino acid sequence of SEQ ID NO: 48; and
(ii) a second antigen-binding region (R2) comprising R2-HCVR comprising the amino acid sequence of SEQ ID NO: 94; and R2-LCVR comprising the amino acid sequence of SEQ ID NO: 48.

32. The bispecific antigen-binding molecule of claim 1, wherein:
(a) the first antigen-binding arm comprises a HCVR comprising the amino acid sequence of SEQ ID NO: 40 and a LCVR comprising the amino acid sequence of SEQ ID NO: 48; and
(b) the second antigen-binding arm comprises:
(i) a first antigen-binding region (R1) comprising R1-HCVR comprising the amino acid sequence of SEQ ID NOs: 62; and R1-LCVR comprising the amino acid sequence of SEQ ID NO: 48; and
(ii) a second antigen-binding region (R2) comprising R2-HCVR comprising the amino acid sequence of SEQ ID NO: 62; and R2-LCVR comprising the amino acid sequence of SEQ ID NO: 48.

33. The bispecific antigen-binding molecule of claim 1, wherein the molecule is a bispecific antibody.

34. The bispecific antigen-binding molecule of claim 33, wherein the bispecific antibody comprises a heavy chain constant region of IgG1 or IgG4 isotype.

35. The bispecific antigen-binding molecule of claim 33, wherein the bispecific antibody comprises a first heavy chain comprising the HCVR of the first antigen-binding arm, and a second heavy chain comprising R1-HCVR and R2-HCVR of the second antigen-binding arm, wherein the second heavy chain comprises the mutations H435R and Y436F (EU numbering).

36. The bispecific antigen-binding molecule of claim 33 comprising a first heavy chain comprising the HCVR of the first antigen-binding arm paired with a light chain comprising the LCVR of the first antigen-binding arm, wherein the first heavy chain comprises the amino acid sequence of SEQ ID NO: 58 and the light chain comprises the amino acid sequence of SEQ ID NO: 60.

37. The bispecific antigen-binding molecule of claim 36 comprising a second heavy chain comprising R1-HCVR and R2-HCVR of the second antigen-binding arm paired with a first light chain comprising R1-LCVR and a second light chain comprising R2-LCVR, wherein the second heavy chain comprises the amino acid sequence selected from the group consisting of SEQ ID NO: 56, 70, 80, 82, and 84; the first light chain comprises the amino acid sequence of SEQ ID NO: 60, and the second light chain comprises the amino acid sequence of SEQ ID NO: 60.

38. The bispecific antigen-binding molecule of claim 33, wherein:
(a) the first antigen-binding arm that specifically binds human CD38 comprises a heavy chain comprising the sequence of SEQ ID NO: 58, and a light chain comprising the sequence of SEQ ID NO: 60; and
(b) the second antigen-binding arm that specifically binds human 4-1BB comprises a heavy chain comprising the sequence of SEQ ID NO: 56, a first light chain comprising the sequence of SEQ ID NO: 60, and a second light chain comprising the sequence of SEQ ID NO: 60.

39. The bispecific antigen-binding molecule of claim 33, wherein:
(a) the first antigen-binding arm that specifically binds human CD38 comprises a heavy chain comprising the sequence of SEQ ID NO: 58, and a light chain comprising the sequence of SEQ ID NO: 60; and
(b) the second antigen-binding arm that specifically binds human 4-1BB comprises a heavy chain comprising the sequence of SEQ ID NO: 70, a first light chain comprising the sequence of SEQ ID NO: 60, and a second light chain comprising the sequence of SEQ ID NO: 60.

40. The bispecific antigen-binding molecule of claim 33, wherein:
(a) the first antigen-binding arm that specifically binds human CD38 comprises a heavy chain comprising the sequence of SEQ ID NO: 58, and a light chain comprising the sequence of SEQ ID NO: 60; and
(b) the second antigen-binding arm that specifically binds human 4-1BB comprises a heavy chain comprising the sequence of SEQ ID NO: 80, a first light chain comprising the sequence of SEQ ID NO: 60, and a second light chain comprising the sequence of SEQ ID NO: 60.

41. The bispecific antigen-binding molecule of claim 33, wherein:
(a) the first antigen-binding arm that specifically binds human CD38 comprises a heavy chain comprising the sequence of SEQ ID NO: 58, and a light chain comprising the sequence of SEQ ID NO: 60; and
(b) the second antigen-binding arm that specifically binds human 4-1BB comprises a heavy chain comprising the sequence of SEQ ID NO: 82, a first light chain comprising the sequence of SEQ ID NO: 60, and a second light chain comprising the sequence of SEQ ID NO: 60.

42. The bispecific antigen-binding molecule of claim 33, wherein:
(a) the first antigen-binding arm that specifically binds human CD38 comprises a heavy chain comprising the sequence of SEQ ID NO: 58, and a light chain comprising the sequence of SEQ ID NO: 60; and
(b) the second antigen-binding arm that specifically binds human 4-1BB comprises a heavy chain comprising the sequence of SEQ ID NO: 84, a first light chain comprising the sequence of SEQ ID NO: 60, and a second light chain comprising the sequence of SEQ ID NO: 60.

43. A bispecific antigen-binding molecule comprising a first antigen binding arm that binds specifically to CD38 and a second antigen-binding arm that binds specifically to 4-1BB, wherein:
(a) the first antigen binding arm comprises three CDRs of a HCVR comprising the amino acid sequence of SEQ ID NO: 40, and three CDRs of LCVR comprising the amino acid sequence of SEQ ID NO: 48; and
(b) the second antigen-binding arm comprises:
(i) a first antigen-binding region (R1) comprising three CDRs of a HCVR (R1-HCVR) comprising the amino acid sequence of SEQ ID NO: 62, and three CDRs of a LCVR (R1-LCVR) comprising the amino acid sequence of SEQ ID NO: 48; and
(ii) a second antigen-binding region (R2) comprising three CDRs of a HCVR (R2-HCVR) comprising the amino acid sequence of SEQ ID NO: 62, and three CDRs of a LCVR (R2-LCVR) comprising the amino acid sequence of SEQ ID NO: 48.

44. The bispecific antigen-binding molecule of claim 43, wherein the molecule is a bispecific antibody.

45. The bispecific antigen-binding molecule of claim 44, wherein the bispecific antibody comprises a first heavy chain comprising the HCVR of the first antigen-binding arm, wherein the first heavy chain is paired with a light chain comprising the LCVR of the first antigen-binding arm, wherein the first heavy chain comprises the amino acid sequence of SEQ ID NO: 58 and the light chain comprises the amino acid sequence of SEQ ID NO: 60.

46. The bispecific antigen-binding molecule of claim 45, wherein the bispecific antibody comprises a second heavy chain comprising R1-HCVR and R2-HCVR of the second antigen-binding arm, wherein the second heavy chain is paired with a first light chain comprising R1-LCVR, and a second light chain comprising R2-LCVR, wherein the second heavy chain comprises the amino acid sequence of SEQ ID NO: 70, 82 or 84, the first light chain comprises the amino acid sequence of SEQ ID NO: 60, and the second light chain comprises the amino acid sequence of SEQ ID NO: 60.

47. The bispecific antigen-binding molecule of claim 1, wherein:
(a) the first antigen-binding arm that specifically binds human CD38 comprises a heavy chain comprising the sequence of SEQ ID NO: 58, and a light chain comprising the sequence of SEQ ID NO: 60; and
(b) the second antigen-binding arm that specifically binds human 4-1BB comprises:
(i) a heavy chain comprising the sequence of SEQ ID NO: 70, a first light chain comprising the sequence of SEQ ID NO: 60, and a second light chain comprising the sequence of SEQ ID NO: 60;
(ii) a heavy chain comprising the sequence of SEQ ID NO: 82, a first light chain comprising the sequence of SEQ ID NO: 60, and a second light chain comprising the sequence of SEQ ID NO: 60; or
(iii) a heavy chain comprising the sequence of SEQ ID NO: 84, a first light chain comprising the sequence of SEQ ID NO: 60, and a second light chain comprising the sequence of SEQ ID NO: 60.

48. A pharmaceutical composition comprising the bispecific antigen-binding molecule of claim 1, and a pharmaceutically acceptable carrier.

49. An isolated nucleic acid molecule comprising a nucleic acid sequence encoding for HCVR of the first antigen-binding arm of the bispecific antigen-binding molecule of claim 1.

50. An isolated nucleic acid molecule comprising a nucleic acid sequence encoding for R1-HCVR and R2-HCVR of the second antigen-binding arm of the bispecific antigen-binding molecule of claim 1.

51. An isolated nucleic acid molecule comprising a nucleic acid sequence encoding for LCVR of the bispecific antigen-binding molecule of claim 1.

52. An expression vector containing the isolated nucleic acid molecule of claim 1.

53. A host cell containing the expression vector of claim 52.

54. The host cell of claim 53, wherein the host cell is E. coli or CHO cell.

55. A method of producing a multispecific antigen binding molecule, the method comprising growing the host cell of claim 53 under conditions permitting production of the multispecific antigen binding molecule, wherein the host cell comprises a first nucleic acid molecule comprising a nucleic acid sequence encoding a heavy chain variable region (HCVR) of a multispecific antigen binding molecule antigen binding arm A1, a second nucleic acid molecule comprising a nucleic acid sequence encoding heavy chain variable regions (HCVRs) of a multispecific antigen binding molecule antigen binding arm A2, and a third nucleic acid molecule comprising a nucleic acid sequence encoding a common light chain variable region (LCVR).

56. (canceled)

57. A method of inhibiting growth of a plasma cell tumor in a subject, comprising administering a multispecific antigen binding molecule of claim 1 to the subject.

58. (canceled)

59. A method of inhibiting growth of a tumor in a subject, the method comprising administering a multispecific antigen binding molecule of claim 1 to the subject, wherein the tumor is selected from the group consisting of multiple myeloma, lymphoma, B-cell leukemia, hepatocellular carcinoma, non-small cell lung cancer, melanoma, pancreatic ductal adenocarcinoma, glioma, or breast cancer, or another cancer characterized in part by having CD38+ cells.

60. A method of treating a patient suffering from a CD38+ tumor and/or a BCMA-expressing tumor comprising administering a multispecific antigen binding molecule of claim 1 to the subject.

61.-76. (canceled)

77. A pharmaceutical composition comprising the bispecific antigen-binding molecule of claim 43, and a pharmaceutically acceptable carrier.


SEQUENCE LISTING (102 sequences)

SEQ ID NO: 1 - DNA, 348 bp, synthetic construct
  misc_feature 1..348: note = Synthetic
  source 1..348: mol_type = other DNA
caggtgcagctggtgcagtctggggctgaagtgaagaagcctgggtcctcggtgaaggtctcctgtaaggcttctggaggccccttcagaagttcttctttcagctgggtgcgacaggccccaggacaagggcttgaatggatgggagggatcatccctattttaggtaaaacaaactatgcacagaagttccagggcagaatcacgattgtcacggacgaatccacgaccacagtctacatggaactgagcagcctgagatctgaggacacggccgttttttactgtgtgagaggcagctcgctctttgactattggggccagggaaccctggtcaccgtctcctca

SEQ ID NO: 2 - protein, 116 aa, synthetic construct
  REGION 1..116: note = Synthetic
QVQLVQSGAEVKKPGSSVKVSCKASGGPFRSSSFSWVRQAPGQGLEWMGGIIPILGKTNYAQKFQGRITIVTDESTTTVYMELSSLRSEDTAVFYCVRGSSLFDYWGQGTLVTVSS

SEQ ID NO: 3 - DNA, 24 bp, synthetic construct
  misc_feature 1..24: note = Synthetic
  source 1..24: mol_type = other DNA
ggaggccccttcagaagttcttct

SEQ ID NO: 4 - protein, 8 aa, synthetic construct
  REGION 1..8: note = Synthetic
GGPFRSSS

SEQ ID NO: 5 - DNA, 24 bp, synthetic construct
  misc_feature 1..24: note = Synthetic
  source 1..24: mol_type = other DNA
atcatccctattttaggtaaaaca

SEQ ID NO: 6 - protein, 8 aa, synthetic construct
  REGION 1..8: note = Synthetic
IIPILGKT

SEQ ID NO: 7 - DNA, 27 bp, synthetic construct
  misc_feature 1..27: note = Synthetic
  source 1..27: mol_type = other DNA
gtgagaggcagctcgctctttgactat

SEQ ID NO: 8 - protein, 9 aa, synthetic construct
  REGION 1..9: note = Synthetic
VRGSSLFDY

SEQ ID NO: 9 - DNA, 357 bp, synthetic construct
  misc_feature 1..357: note = Synthetic
  source 1..357: mol_type = other DNA
caggttcagctggtgcagtctggagctgaggtgaagaagcctggggcctcagtgaaggtctcctgcaaggcttctgcttacacctttaccagctatggtttcagctgggtgcgacaggcccctggacaagggcttgagtggatgggatggatcagcacttccaatggttacacaaactatgcacagaagatccaggacagagtcaccatgaccacagacacatccacgagcacagcctacatggagctgaggagcctgagatctgacgacacggccgtgtattattgtgcgagcctctctaactgggtatctttctttgactactggggccagggaaccctggtcaccgtctcctca

SEQ ID NO: 10 - protein, 119 aa, synthetic construct
  REGION 1..119: note = Synthetic
QVQLVQSGAEVKKPGASVKVSCKASAYTFTSYGFSWVRQAPGQGLEWMGWISTSNGYTNYAQKIQDRVTMTTDTSTSTAYMELRSLRSDDTAVYYCASLSNWVSFFDYWGQGTLVTVSS

SEQ ID NO: 11 - DNA, 24 bp, synthetic construct
  misc_feature 1..24: note = Synthetic
  source 1..24: mol_type = other DNA
gcttacacctttaccagctatggt

SEQ ID NO: 12 - protein, 8 aa, synthetic construct
  REGION 1..8: note = Synthetic
AYTFTSYG

SEQ ID NO: 13 - DNA, 24 bp, synthetic construct
  misc_feature 1..24: note = Synthetic
  source 1..24: mol_type = other DNA
atcagcacttccaatggttacaca

SEQ ID NO: 14 - protein, 8 aa, synthetic construct
  REGION 1..8: note = Synthetic
ISTSNGYT

SEQ ID NO: 15 - DNA, 36 bp, synthetic construct
  misc_feature 1..36: note = Synthetic
  source 1..36: mol_type = other DNA
gcgagcctctctaactgggtatctttctttgactac

SEQ ID NO: 16 - protein, 12 aa, synthetic construct
  REGION 1..12: note = Synthetic
ASLSNWVSFFDY

SEQ ID NO: 17 - DNA, 324 bp, synthetic construct
  misc_feature 1..324: note = Synthetic
  source 1..324: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaag

SEQ ID NO: 18 - protein, 108 aa, synthetic construct
  REGION 1..108: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 19 - DNA, 21 bp, synthetic construct
  misc_feature 1..21: note = Synthetic
  source 1..21: mol_type = other DNA
cagtcagtctctagctcttat

SEQ ID NO: 20 - protein, 7 aa, synthetic construct
  REGION 1..7: note = Synthetic
QSVSSSY

SEQ ID NO: 21-22 - skipped (2 entries)

SEQ ID NO: 23 - DNA, 27 bp, synthetic construct
  misc_feature 1..27: note = Synthetic
  source 1..27: mol_type = other DNA
caacagtacggaagcagcccgtggacg

SEQ ID NO: 24 - protein, 9 aa, synthetic construct
  REGION 1..9: note = Synthetic
QQYGSSPWT

SEQ ID NO: 25 - DNA, 1329 bp, synthetic construct
  misc_feature 1..1329: note = Synthetic
  source 1..1329: mol_type = other DNA
caggtgcagctggtgcagtctggggctgaagtgaagaagcctgggtcctcggtgaaggtctcctgtaaggcttctggaggccccttcagaagttcttctttcagctgggtgcgacaggccccaggacaagggcttgaatggatgggagggatcatccctattttaggtaaaacaaactatgcacagaagttccagggcagaatcacgattgtcacggacgaatccacgaccacagtctacatggaactgagcagcctgagatctgaggacacggccgttttttactgtgtgagaggcagctcgctctttgactattggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 26 - protein, 442 aa, synthetic construct
  REGION 1..442: note = Synthetic
QVQLVQSGAEVKKPGSSVKVSCKASGGPFRSSSFSWVRQAPGQGLEWMGGIIPILGKTNYAQKFQGRITIVTDESTTTVYMELSSLRSEDTAVFYCVRGSSLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - DNA, 1338 bp, synthetic construct
  misc_feature 1..1338: note = Synthetic
  source 1..1338: mol_type = other DNA
caggttcagctggtgcagtctggagctgaggtgaagaagcctggggcctcagtgaaggtctcctgcaaggcttctgcttacacctttaccagctatggtttcagctgggtgcgacaggcccctggacaagggcttgagtggatgggatggatcagcacttccaatggttacacaaactatgcacagaagatccaggacagagtcaccatgaccacagacacatccacgagcacagcctacatggagctgaggagcctgagatctgacgacacggccgtgtattattgtgcgagcctctctaactgggtatctttctttgactactggggccagggaaccctggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 28 - protein, 445 aa, synthetic construct
  REGION 1..445: note = Synthetic
QVQLVQSGAEVKKPGASVKVSCKASAYTFTSYGFSWVRQAPGQGLEWMGWISTSNGYTNYAQKIQDRVTMTTDTSTSTAYMELRSLRSDDTAVYYCASLSNWVSFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 29 - DNA, 648 bp, synthetic construct
  misc_feature 1..648: note = Synthetic
  source 1..648: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 30 - protein, 215 aa, synthetic construct
  REGION 1..215: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 381 bp, synthetic construct
  misc_feature 1..381: note = Synthetic
  source 1..381: mol_type = other DNA
gaagtgcagctggtggagtctgggggaggcttggtacagcctggcaggtccctgagactctcctgtgcagcctctggatacacctttgatgattatgccatgcactgggtccggcaagctccagggaagggcctggagtgggtctcaggtattagttggattagtgatgttataggctatgcggactctgtgaagggccgattcaccatctccagagacaacgccaagaactccctgtatctgcaaatgaacagtctgagagctgaggacacggccttgtattactgtgcaaaagataacggagggattatagtggctacccactattactcctacggtatggacgtctggggccaagggaccacggtcaccgtctcctca

SEQ ID NO: 32 - protein, 127 aa, synthetic construct
  REGION 1..127: note = Synthetic
EVQLVESGGGLVQPGRSLRLSCAASGYTFDDYAMHWVRQAPGKGLEWVSGISWISDVIGYADSVKGRFTISRDNAKNSLYLQMNSLRAEDTALYYCAKDNGGIIVATHYYSYGMDVWGQGTTVTVSS

SEQ ID NO: 33 - DNA, 24 bp, synthetic construct
  misc_feature 1..24: note = Synthetic
  source 1..24: mol_type = other DNA
ggatacacctttgatgattatgcc

SEQ ID NO: 34 - protein, 8 aa, synthetic construct
  REGION 1..8: note = Synthetic
GYTFDDYA

SEQ ID NO: 35 - DNA, 24 bp, synthetic construct
  misc_feature 1..24: note = Synthetic
  source 1..24: mol_type = other DNA
attagttggattagtgatgttata

SEQ ID NO: 36 - protein, 8 aa, synthetic construct
  REGION 1..8: note = Synthetic
ISWISDVI

SEQ ID NO: 37 - DNA, 60 bp, synthetic construct
  misc_feature 1..60: note = Synthetic
  source 1..60: mol_type = other DNA
gcaaaagataacggagggattatagtggctacccactattactcctacggtatggacgtc

SEQ ID NO: 38 - protein, 20 aa, synthetic construct
  REGION 1..20: note = Synthetic
AKDNGGIIVATHYYSYGMDV

SEQ ID NO: 39 - DNA, 372 bp, synthetic construct
  misc_feature 1..372: note = Synthetic
  source 1..372: mol_type = other DNA
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaaaagtgataacataggctatgcggactctgtgaagggccgattcaccatttccagagacaacgccaagaactccctgtatctgcaaatgaacagtctgagagctgaggacacggccttgtattactgtgcaaaagcccttgggggctggaagttcgactactattacggtatggacgtttggggccaagggaccacggtcaccgtctcctca

SEQ ID NO: 40 - protein, 124 aa, synthetic construct
  REGION 1..124: note = Synthetic
EVQLVESGGGLVQPGRSLRLSCAASGFTFDDYAMHWVRQAPGKGLEWVSGISWKSDNIGYADSVKGRFTISRDNAKNSLYLQMNSLRAEDTALYYCAKALGGWKFDYYYGMDVWGQGTTVTVSS

SEQ ID NO: 41 - DNA, 24 bp, synthetic construct
  misc_feature 1..24: note = Synthetic
  source 1..24: mol_type = other DNA
ggattcacctttgatgattatgcc

SEQ ID NO: 42 - protein, 8 aa, synthetic construct
  REGION 1..8: note = Synthetic
GFTFDDYA

SEQ ID NO: 43 - DNA, 24 bp, synthetic construct
  misc_feature 1..24: note = Synthetic
  source 1..24: mol_type = other DNA
attagttggaaaagtgataacata

SEQ ID NO: 44 - protein, 8 aa, synthetic construct
  REGION 1..8: note = Synthetic
ISWKSDNI

SEQ ID NO: 45 - DNA, 51 bp, synthetic construct
  misc_feature 1..51: note = Synthetic
  source 1..51: mol_type = other DNA
gcaaaagcccttgggggctggaagttcgactactattacggtatggacgtt

SEQ ID NO: 46 - protein, 17 aa, synthetic construct
  REGION 1..17: note = Synthetic
AKALGGWKFDYYYGMDV

SEQ ID NO: 47 - DNA, 324 bp, synthetic construct
  misc_feature 1..324: note = Synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 48 - protein, 108 aa, synthetic construct
  REGION 1..108: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 49 - DNA, 18 bp, synthetic construct
  misc_feature 1..18: note = Synthetic
  source 1..18: mol_type = other DNA
cagagcattagcagctat

SEQ ID NO: 50 - protein, 6 aa, synthetic construct
  REGION 1..6: note = Synthetic
QSISSY

SEQ ID NO: 51-52 - skipped (2 entries)

SEQ ID NO: 53 - DNA, 30 bp, synthetic construct
  misc_feature 1..30: note = Synthetic
  source 1..30: mol_type = other DNA
caacagagttacagtacccctccgatcacc

SEQ ID NO: 54 - protein, 10 aa, synthetic construct
  REGION 1..10: note = Synthetic
QQSYSTPPIT

SEQ ID NO: 55 - DNA, 2085 bp, synthetic construct
  misc_feature 1..2085: note = Synthetic
  source 1..2085: mol_type = other DNA
caggttcagctggtgcagtctggtcctgaggtgaagaaccctggggccgcagtgaaggtctcctgcaaggcctctggttacacctttaccaggtttggtatcagctgggtgcgacaggcccctggacaagggcttgagtggatgggatggatcagcgttgacaatggtaacgcaaactatgcacagaggttccagggcagagtcaccatgaccacagacacttccacgagcacagcctacatggagctgaggagcctgagatctgacgacacggccgtatattactgtgcgagagaatggagtattgcagccccaggataccttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccaggtggaggcggtagtggcggaggcggaagtggtggaggaggctcagaagtgcagctggtggagtctgggggaggcttggtacagcctggcaggtccctgagactctcctgtgcagcctctggatacacctttgatgattatgccatgcactgggtccggcaagctccagggaagggcctggagtgggtctcaggtattagttggattagtgatgttataggctatgcggactctgtgaagggccgattcaccatctccagagacaacgccaagaactccctgtatctgcaaatgaacagtctgagagctgaggacacggccttgtattactgtgcaaaagataacggagggattatagtggctacccactattactcctacggtatggacgtctggggccaagggaccacggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 56 - protein, 694 aa, synthetic construct
  REGION 1..694: note = Synthetic
QVQLVQSGPEVKNPGAAVKVSCKASGYTFTRFGISWVRQAPGQGLEWMGWISVDNGNANYAQRFQGRVTMTTDTSTSTAYMELRSLRSDDTAVYYCAREWSIAAPGYLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPGGGGSGGGGSGGGGSEVQLVESGGGLVQPGRSLRLSCAASGYTFDDYAMHWVRQAPGKGLEWVSGISWISDVIGYADSVKGRFTISRDNAKNSLYLQMNSLRAEDTALYYCAKDNGGIIVATHYYSYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 57 - DNA, 1353 bp, synthetic construct
  misc_feature 1..1353: note = Synthetic
  source 1..1353: mol_type = other DNA
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaaaagtgataacataggctatgcggactctgtgaagggccgattcaccatttccagagacaacgccaagaactccctgtatctgcaaatgaacagtctgagagctgaggacacggccttgtattactgtgcaaaagcccttgggggctggaagttcgactactattacggtatggacgtttggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 58 - protein, 450 aa, synthetic construct
  REGION 1..450: note = Synthetic
EVQLVESGGGLVQPGRSLRLSCAASGFTFDDYAMHWVRQAPGKGLEWVSGISWKSDNIGYADSVKGRFTISRDNAKNSLYLQMNSLRAEDTALYYCAKALGGWKFDYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - DNA, 648 bp, synthetic construct
  misc_feature 1..648: note = Synthetic
  source 1..648: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 60 - protein, 215 aa, synthetic construct
  REGION 1..215: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - DNA, 381 bp, synthetic construct
  misc_feature 1..381: note = Synthetic
  source 1..381: mol_type = other DNA
gaagtgcagctggtggagtctgggggaggcctggtacagcctggcaggtccctgagactctcctgtggagcctctggattcacctttgatgattatgtcatgcactgggtccggcaaattccagggaagggcctggagtgggtcgcaggtattagttggaatagtggtagtataggctatgcggactctgtgaagggccgattcaccatctccagagacaacgccaagaactccctgtatctgcaaatgaacagtctgagagctgaggacacggccttgtatttctgtgcaaaagatcatattgacccgtatagcagcagcctctactactactatggtatggacgtctggggccaagggaccacggtcaccgtctcctca

SEQ ID NO: 62 - protein, 127 aa, synthetic construct
  REGION 1..127: note = Synthetic
EVQLVESGGGLVQPGRSLRLSCGASGFTFDDYVMHWVRQIPGKGLEWVAGISWNSGSIGYADSVKGRFTISRDNAKNSLYLQMNSLRAEDTALYFCAKDHIDPYSSSLYYYYGMDVWGQGTTVTVSS

SEQ ID NO: 63 - DNA, 24 bp, synthetic construct
  misc_feature 1..24: note = Synthetic
  source 1..24: mol_type = other DNA
ggattcacctttgatgattatgtc

SEQ ID NO: 64 - protein, 8 aa, synthetic construct
  REGION 1..8: note = Synthetic
GFTFDDYV

SEQ ID NO: 65 - DNA, 24 bp, synthetic construct
  misc_feature 1..24: note = Synthetic
  source 1..24: mol_type = other DNA
attagttggaatagtggtagtata

SEQ ID NO: 66 - protein, 8 aa, synthetic construct
  REGION 1..8: note = Synthetic
ISWNSGSI

SEQ ID NO: 67 - DNA, 60 bp, synthetic construct
  misc_feature 1..60: note = Synthetic
  source 1..60: mol_type = other DNA
gcaaaagatcatattgacccgtatagcagcagcctctactactactatggtatggacgtc

SEQ ID NO: 68 - protein, 20 aa, synthetic construct
  REGION 1..20: note = Synthetic
AKDHIDPYSSSLYYYYGMDV

SEQ ID NO: 69 - DNA, 2103 bp, synthetic construct
  misc_feature 1..2103: note = Synthetic
  source 1..2103: mol_type = other DNA
gaagtgcagctggtggagtctgggggaggcctggtacagcctggcaggtccctgagactctcctgtggagcctctggattcacctttgatgattatgtcatgcactgggtccggcaaattccagggaagggcctggagtgggtcgcaggtattagttggaatagtggtagtataggctatgcggactctgtgaagggccgattcaccatctccagagacaacgccaagaactccctgtatctgcaaatgaacagtctgagagctgaggacacggccttgtatttctgtgcaaaagatcatattgacccgtatagcagcagcctctactactactatggtatggacgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccaggtggaggcggtagtggcggaggcggaagtggtggaggaggctcagaagtgcagctggtggagtctgggggaggcctggtacagcctggcaggtccctgagactctcctgtggagcctctggattcacctttgatgattatgtcatgcactgggtccggcaaattccagggaagggcctggagtgggtcgcaggtattagttggaatagtggtagtataggctatgcggactctgtgaagggccgattcaccatctccagagacaacgccaagaactccctgtatctgcaaatgaacagtctgagagctgaggacacggccttgtatttctgtgcaaaagatcatattgacccgtatagcagcagcctctactactactatggtatggacgtctggggccaagggaccacggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 70 - protein, 700 aa, synthetic construct
  REGION 1..700: note = Synthetic
EVQLVESGGGLVQPGRSLRLSCGASGFTFDDYVMHWVRQIPGKGLEWVAGISWNSGSIGYADSVKGRFTISRDNAKNSLYLQMNSLRAEDTALYFCAKDHIDPYSSSLYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPGGGGSGGGGSGGGGSEVQLVESGGGLVQPGRSLRLSCGASGFTFDDYVMHWVRQIPGKGLEWVAGISWNSGSIGYADSVKGRFTISRDNAKNSLYLQMNSLRAEDTALYFCAKDHIDPYSSSLYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 71 - DNA, 342 bp, synthetic construct
  misc_feature 1..342: note = Synthetic
  source 1..342: mol_type = other DNA
caggtgcagctggtggagtctgggggagacgtggtccagcctgggaggtccctgagactctcctgtacagcgtctggattcaccttcagtagctttggcatgcactgggtccgccaggctccaggcaaggggctggagtgggtgacatttttatcgtatgatggaagttataaatactatgcagactccgtgaagggccgattcaccatctccagagacaattccaagaacacgctatatctgcaaatgaacagcctgagagccgaagacacggctatcttttactgtgcgagggactggcttgactactggggccagggaaccctggtcaccgtctcctca

SEQ ID NO: 72 - protein, 114 aa, synthetic construct
  REGION 1..114: note = Synthetic
QVQLVESGGDVVQPGRSLRLSCTASGFTFSSFGMHWVRQAPGKGLEWVTFLSYDGSYKYYADSVKGRFTISRDNSKNTLYLQMNSLRAEDTAIFYCARDWLDYWGQGTLVTVSS

SEQ ID NO: 73 - DNA, 24 bp, synthetic construct
  misc_feature 1..24: note = Synthetic
  source 1..24: mol_type = other DNA
ggattcaccttcagtagctttggc

SEQ ID NO: 74 - protein, 8 aa, synthetic construct
  REGION 1..8: note = Synthetic
GFTFSSFG

SEQ ID NO: 75 - DNA, 24 bp, synthetic construct
  misc_feature 1..24: note = Synthetic
  source 1..24: mol_type = other DNA
ttatcgtatgatggaagttataaa

SEQ ID NO: 76 - protein, 8 aa, synthetic construct
  REGION 1..8: note = Synthetic
LSYDGSYK

SEQ ID NO: 77 - DNA, 21 bp, synthetic construct
  misc_feature 1..21: note = Synthetic
  source 1..21: mol_type = other DNA
gcgagggactggcttgactac

SEQ ID NO: 78 - protein, 7 aa, synthetic construct
  REGION 1..7: note = Synthetic
ARDWLDY

SEQ ID NO: 79 - DNA, 2046 bp, synthetic construct
  misc_feature 1..2046: note = Synthetic
  source 1..2046: mol_type = other DNA
gaggtgcagctggtggagtctgggggaggcttggtacagcctggagggtccctgagactcccctgtgcagcctctggattcacctttagtagttatgccatgagttgggtccgccaggctccaggcaaggggctggagtgggtcgcagatattagtggtagtggtggtgatacatattatgtagactccgtgaagggccggttcaccatctccagagacaattccaagaacacgctgtatctgcaaatgaacagcctgagaatcgaagacacggccgtgtattactgtgcgaaaggccggtggcactggaactacgaaggttttgatatctggggccaagggacaatggtcaccgtctcttcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccaggtggaggcggtagtggcggaggcggaagtggtggaggaggctcacaggtgcagctggtggagtctgggggagacgtggtccagcctgggaggtccctgagactctcctgtacagcgtctggattcaccttcagtagctttggcatgcactgggtccgccaggctccaggcaaggggctggagtgggtgacatttttatcgtatgatggaagttataaatactatgcagactccgtgaagggccgattcaccatctccagagacaattccaagaacacgctatatctgcaaatgaacagcctgagagccgaagacacggctatcttttactgtgcgagggactggcttgactactggggccagggaaccctggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 80 - protein, 681 aa, synthetic construct
  REGION 1..681: note = Synthetic
EVQLVESGGGLVQPGGSLRLPCAASGFTFSSYAMSWVRQAPGKGLEWVADISGSGGDTYYVDSVKGRFTISRDNSKNTLYLQMNSLRIEDTAVYYCAKGRWHWNYEGFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPGGGGSGGGGSGGGGSQVQLVESGGDVVQPGRSLRLSCTASGFTFSSFGMHWVRQAPGKGLEWVTFLSYDGSYKYYADSVKGRFTISRDNSKNTLYLQMNSLRAEDTAIFYCARDWLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 81 - DNA, 2082 bp, synthetic construct
  misc_feature 1..2082: note = Synthetic
  source 1..2082: mol_type = other DNA
gaagtgcagctggtggagtctgggggaggcctggtacagcctggcaggtccctgagactctcctgtggagcctctggattcacctttgatgattatgtcatgcactgggtccggcaaattccagggaagggcctggagtgggtcgcaggtattagttggaatagtggtagtataggctatgcggactctgtgaagggccgattcaccatctccagagacaacgccaagaactccctgtatctgcaaatgaacagtctgagagctgaggacacggccttgtatttctgtgcaaaagatcatattgacccgtatagcagcagcctctactactactatggtatggacgtctggggccaagggaccacggtcaccgtctcctcagcatctacaaaagggccatccgttttccctctggcaccctgttcaagatctacctctgaatctacagccgctctcggatgcctcgtgaaagactatttcccagaacccgtcaccgtttcatggaacagcggcgccctcacatccggcgtgcatacctttcctgcagtgctccaatcttccggtctgtattctctgtctagtgtcgtgactgttccatcatcttccctcggaactaaaacctatacctgtaacgttgaccataagccctctaacactaaggtggataaaagagtcggcggcggtggatctggaggcggtggaagcggtggcgggggaagtgaagttcaactcgttgagtcaggaggaggactcgtacaaccaggaagatctttgcgtctgtcatgcggtgcttctggatttacatttgatgattacgtaatgcattgggtcagacaaatcccaggtaaaggactggaatgggttgccggaattagttggaacagtggctctataggatatgcagattccgtcaaaggtcgattcactatatctcgtgataacgctaaaaactctctctatctccaaatgaatagccttcgcgctgaagatactgccctttacttttgcgctaaagaccatatagacccatatagttcctctctgtactactactatggcatggacgtctggggccaaggtacaactgttactgtgtcaagtgcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 82 - protein, 693 aa, synthetic construct
  REGION 1..693: note = Synthetic
EVQLVESGGGLVQPGRSLRLSCGASGFTFDDYVMHWVRQIPGKGLEWVAGISWNSGSIGYADSVKGRFTISRDNAKNSLYLQMNSLRAEDTALYFCAKDHIDPYSSSLYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVGGGGSGGGGSGGGGSEVQLVESGGGLVQPGRSLRLSCGASGFTFDDYVMHWVRQIPGKGLEWVAGISWNSGSIGYADSVKGRFTISRDNAKNSLYLQMNSLRAEDTALYFCAKDHIDPYSSSLYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 83 - DNA, 2088 bp, synthetic construct
  misc_feature 1..2088: note = Synthetic
  source 1..2088: mol_type = other DNA
gaagtgcagctggtggagtctgggggaggcctggtacagcctggcaggtccctgagactctcctgtggagcctctggattcacctttgatgattatgtcatgcactgggtccggcaaattccagggaagggcctggagtgggtcgcaggtattagttggaatagtggtagtataggctatgcggactctgtgaagggccgattcaccatctccagagacaacgccaagaactccctgtatctgcaaatgaacagtctgagagctgaggacacggccttgtatttctgtgcaaaagatcatattgacccgtatagcagcagcctctactactactatggtatggacgtctggggccaagggaccacggtcaccgtctcctcagcatctacaaaagggccatccgttttccctctggcaccctgttcaagatctacctctgaatctacagccgctctcggatgcctcgtgaaagactatttcccagaacccgtcaccgtttcatggaacagcggcgccctcacatccggcgtgcatacctttcctgcagtgctccaatcttccggtctgtattctctgtctagtgtcgtgactgttccatcatcttccctcggaactaaaacctatacctgtaacgttgaccataagccctctaacactaaggtggataaaagagtcgaatccaagtacggccctccaggcggcggtggatctggaggcggtggaagcgaagttcaactcgttgagtcaggaggaggactcgtacaaccaggaagatctttgcgtctgtcatgcggtgcttctggatttacatttgatgattacgtaatgcattgggtcagacaaatcccaggtaaaggactggaatgggttgccggaattagttggaacagtggctctataggatatgcagattccgtcaaaggtcgattcactatatctcgtgataacgctaaaaactctctctatctccaaatgaatagccttcgcgctgaagatactgccctttacttttgcgctaaagaccatatagacccatatagttcctctctgtactactactatggcatggacgtctggggccaaggtacaactgttactgtgtcaagtgcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 84 - protein, 695 aa, synthetic construct
  REGION 1..695: note = Synthetic
EVQLVESGGGLVQPGRSLRLSCGASGFTFDDYVMHWVRQIPGKGLEWVAGISWNSGSIGYADSVKGRFTISRDNAKNSLYLQMNSLRAEDTALYFCAKDHIDPYSSSLYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPGGGGSGGGGSEVQLVESGGGLVQPGRSLRLSCGASGFTFDDYVMHWVRQIPGKGLEWVAGISWNSGSIGYADSVKGRFTISRDNAKNSLYLQMNSLRAEDTALYFCAKDHIDPYSSSLYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 85 - DNA, 363 bp, synthetic construct
  misc_feature 1..363: note = Synthetic
  source 1..363: mol_type = other DNA
caggttcagctggtgcagtctggtcctgaggtgaagaaccctggggccgcagtgaaggtctcctgcaaggcctctggttacacctttaccaggtttggtatcagctgggtgcgacaggcccctggacaagggcttgagtggatgggatggatcagcgttgacaatggtaacgcaaactatgcacagaggttccagggcagagtcaccatgaccacagacacttccacgagcacagcctacatggagctgaggagcctgagatctgacgacacggccgtatattactgtgcgagagaatggagtattgcagccccaggataccttgactactggggccagggaaccctggtcaccgtctcctca

SEQ ID NO: 86 - protein, 121 aa, synthetic construct
  REGION 1..121: note = Synthetic
QVQLVQSGPEVKNPGAAVKVSCKASGYTFTRFGISWVRQAPGQGLEWMGWISVDNGNANYAQRFQGRVTMTTDTSTSTAYMELRSLRSDDTAVYYCAREWSIAAPGYLDYWGQGTLVTVSS

SEQ ID NO: 87 - DNA, 24 bp, synthetic construct
  misc_feature 1..24: note = Synthetic
  source 1..24: mol_type = other DNA
ggttacacctttaccaggtttggt

SEQ ID NO: 88 - protein, 8 aa, synthetic construct
  REGION 1..8: note = Synthetic
GYTFTRFG

SEQ ID NO: 89 - DNA, 24 bp, synthetic construct
  misc_feature 1..24: note = Synthetic
  source 1..24: mol_type = other DNA
atcagcgttgacaatggtaacgca

SEQ ID NO: 90 - protein, 8 aa, synthetic construct
  REGION 1..8: note = Synthetic
ISVDNGNA

SEQ ID NO: 91 - DNA, 42 bp, synthetic construct
  misc_feature 1..42: note = Synthetic
  source 1..42: mol_type = other DNA
gcgagagaatggagtattgcagccccaggataccttgactac

SEQ ID NO: 92 - protein, 14 aa, synthetic construct
  REGION 1..14: note = Synthetic
AREWSIAAPGYLDY

SEQ ID NO: 93 - DNA, 363 bp, synthetic construct
  misc_feature 1..363: note = Synthetic
  source 1..363: mol_type = other DNA
gaggtgcagctggtggagtctgggggaggcttggtacagcctggagggtccctgagactcccctgtgcagcctctggattcacctttagtagttatgccatgagttgggtccgccaggctccaggcaaggggctggagtgggtcgcagatattagtggtagtggtggtgatacatattatgtagactccgtgaagggccggttcaccatctccagagacaattccaagaacacgctgtatctgcaaatgaacagcctgagaatcgaagacacggccgtgtattactgtgcgaaaggccggtggcactggaactacgaaggttttgatatctggggccaagggacaatggtcaccgtctcttca

SEQ ID NO: 94 - protein, 121 aa, synthetic construct
  REGION 1..121: note = Synthetic
EVQLVESGGGLVQPGGSLRLPCAASGFTFSSYAMSWVRQAPGKGLEWVADISGSGGDTYYVDSVKGRFTISRDNSKNTLYLQMNSLRIEDTAVYYCAKGRWHWNYEGFDIWGQGTMVTVSS

SEQ ID NO: 95 - DNA, 24 bp, synthetic construct
  misc_feature 1..24: note = Synthetic
  source 1..24: mol_type = other DNA
ggattcacctttagtagttatgcc

SEQ ID NO: 96 - protein, 8 aa, synthetic construct
  REGION 1..8: note = Synthetic
GFTFSSYA

SEQ ID NO: 97 - DNA, 24 bp, synthetic construct
  misc_feature 1..24: note = Synthetic
  source 1..24: mol_type = other DNA
attagtggtagtggtggtgataca

SEQ ID NO: 98 - protein, 8 aa, synthetic construct
  REGION 1..8: note = Synthetic
ISGSGGDT

SEQ ID NO: 99 - DNA, 42 bp, synthetic construct
  misc_feature 1..42: note = Synthetic
  source 1..42: mol_type = other DNA
gcgaaaggccggtggcactggaactacgaaggttttgatatc

SEQ ID NO: 100 - protein, 14 aa, synthetic construct
  REGION 1..14: note = Synthetic
AKGRWHWNYEGFDI

SEQ ID NO: 101 - protein, 255 aa, synthetic construct
  REGION 1..255: note = Synthetic
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 102 - protein, 491 aa, synthetic construct
  REGION 1..491: note = Synthetic
VPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK